SEQUENCE LISTING (141 sequences)

SEQ ID NO: 1 - protein, 19 aa, synthetic construct
GGGGSGGGGSGGGSGGGGS

SEQ ID NO: 2 - protein, 15 aa, synthetic construct
GGGGSGGGGSGGGGS

SEQ ID NO: 3 - protein, 24 aa, synthetic construct
GGGGSGGGGSGGGGSGGGSGGGGS

SEQ ID NO: 4 - protein, 29 aa, synthetic construct
GGGGSGGGGSGGGGSGGGGSGGGSGGGGS

SEQ ID NO: 5 - protein, 5 aa, synthetic construct
GGGGS

SEQ ID NO: 6 - protein, 10 aa, synthetic construct
GGGGSGGGGS

SEQ ID NO: 7 - protein, 20 aa, synthetic construct
GGGGSGGGGSGGGGSGGGGS

SEQ ID NO: 8 - protein, 20 aa, Homo sapiens
MYRMQLLSCIALSLALVTNS

SEQ ID NO: 9 - protein, 20 aa, Mus musculus
MYSMQLASCVTLTLVLLVNS

SEQ ID NO: 10 - protein, 20 aa, Homo sapiens
METPAQLLFLLLLWLPDTTG

SEQ ID NO: 11 - protein, 20 aa, Mus musculus
METDTLLLWVLLLWVPGSTG

SEQ ID NO: 12 - protein, 21 aa, Homo sapiens
MALPVTALLLPLALLLHAARP

SEQ ID NO: 13 - protein, 18 aa, Homo sapiens
MALPVTALLLPLALLLHA

SEQ ID NO: 14 - protein, 16 aa, Homo sapiens
MKWVTFISLLFSSAYS

SEQ ID NO: 15 - protein, 30 aa, Homo sapiens
MDSKGSSQKGSRLLLLLVVSNLLLCQGVVS

SEQ ID NO: 16 - protein, 11 aa, Homo sapiens
RSKRSRLLHSD

SEQ ID NO: 17 - protein, 19 aa, synthetic construct
ATNFSLLKQAGDVEENPGP

SEQ ID NO: 18 - protein, 18 aa, synthetic construct
EGRGSLLTCGDVEENPGP

SEQ ID NO: 19 - DNA, 123 bp, synthetic construct
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 20 - protein, 39 aa, Homo sapiens
IEVMYPPPYLDNEKSNGTIIHVKGKHLCPSPLFPGPSKP

SEQ ID NO: 21 - protein, 338 aa, synthetic construct
EVQLLESGGGLVQPGGSLRLSCAASGFTFSTYAMSWVRQAPGKGLEWVSAISGRGGSTYYTDSVKGRFTISRDNSKNTVSLQMNSLRAEDTAVYYCAGRGDYYYYYGMDVWGQGTTVTVSAGGGGSGGGGSGGGGSDIVMTQSPLSSPVTLGQPASFSCRSSQSLVYSDGNTYLSWLQQRPGQPPRLLIYKISNRFSGVPDRFSGSGAGTDFTLKISRVEAEDVGVYYCMQSTQFPHTFGQGTKLEIKEQKLISEEDLAAAIEVMYPPPYLDNEKSNGTIIHVKGKHLCPSPLFPGPSKPFWVLVVVGGVLACYSLLVTVAFIIFWVRSKRSRLLHSD

SEQ ID NO: 22 - protein, 325 aa, synthetic construct
EVQLLESGGGLVQPGGSLRLSCAASGFTFSTYAMSWVRQAPGKGLEWVSAISGRGGSTYYTDSVKGRFTISRDNSKNTVSLQMNSLRAEDTAVYYCAGRGDYYYYYGMDVWGQGTTVTVSAGGGGSGGGGSGGGGSDIVMTQSPLSSPVTLGQPASFSCRSSQSLVYSDGNTYLSWLQQRPGQPPRLLIYKISNRFSGVPDRFSGSGAGTDFTLKISRVEAEDVGVYYCMQSTQFPHTFGQGTKLEIKIEVMYPPPYLDNEKSNGTIIHVKGKHLCPSPLFPGPSKPFWVLVVVGGVLACYSLLVTVAFIIFWVRSKRSRLLHSD

SEQ ID NO: 23 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 24 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 25 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 26 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 27 - protein, 364 aa, Homo sapiens
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 28 - protein, 8 aa, synthetic construct
GFTFSTYA

SEQ ID NO: 29 - protein, 8 aa, synthetic construct
ISGRGGST

SEQ ID NO: 30 - protein, 14 aa, synthetic construct
AGRGDYYYYYGMDV

SEQ ID NO: 31 - protein, 11 aa, synthetic construct
QSLVYSDGNTY

SEQ ID NO: 32 - skipped

SEQ ID NO: 33 - protein, 9 aa, synthetic construct
MQSTQFPHT

SEQ ID NO: 34 - protein, 121 aa, synthetic construct
EVQLLESGGGLVQPGGSLRLSCAASGFTFSTYAMSWVRQAPGKGLEWVSAISGRGGSTYYTDSVKGRFTISRDNSKNTVSLQMNSLRAEDTAVYYCAGRGDYYYYYGMDVWGQGTTVTVSA

SEQ ID NO: 35 - DNA, 363 bp, synthetic construct
  source 1..363: mol_type = other DNA
gaggtgcagctgttggagtctgggggaggcttggtccagcctggggggtccctgagactctcctgtgcagcctctggattcacctttagcacctatgccatgagctgggtccgccaggctccagggaaggggctggagtgggtctcagctattagtggtcgtggtggtagcacatactacacagactccgtgaagggccggttcaccatctccagagacaattccaagaacacggtgtctctgcaaatgaacagcctgagagccgaggacacggccgtatattactgtgcgggccggggagattactactactactacggtatggacgtctggggccaagggaccacggtcaccgtctccgca

SEQ ID NO: 36 - protein, 112 aa, synthetic construct
DIVMTQSPLSSPVTLGQPASFSCRSSQSLVYSDGNTYLSWLQQRPGQPPRLLIYKISNRFSGVPDRFSGSGAGTDFTLKISRVEAEDVGVYYCMQSTQFPHTFGQGTKLEIK

SEQ ID NO: 37 - DNA, 336 bp, synthetic construct
  source 1..336: mol_type = other DNA
gatattgtgatgacccagagtccactctcctcacctgtcacccttggacagccggcctccttctcctgcaggtctagtcaaagcctcgtatacagtgatggaaacacctacttgagttggcttcagcagaggccaggccagcctccaagactcctaatttataagatttctaaccggttctctggggtcccagacagattcagtggcagtggggcagggacagatttcacactgaaaatcagcagggtggaagctgaggatgtcggggtttattactgcatgcaatctacacaatttcctcacacttttggccaggggaccaagctggagatcaaa

SEQ ID NO: 38 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 39 - protein, 247 aa, Mus musculus
MASPLTRFLSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 40 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 41 - DNA, 81 bp, synthetic construct
  source 1..81: mol_type = other DNA
ttttgggtgctggtggtggttggtggagtcctggcttgctatagcttgctagtaacagtggcctttattattttctgggtg

SEQ ID NO: 42 - protein, 218 aa, Mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 43 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 44 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - DNA, 336 bp, synthetic construct
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 46 - DNA, 33 bp, synthetic construct
  source 1..33: mol_type = other DNA
aggagtaagaggagcaggctcctgcacagtgac

SEQ ID NO: 47 - protein, 4 aa, synthetic construct
YMNM

SEQ ID NO: 48 - protein, 4 aa, synthetic construct
  VARIANT 3: note = A can be replaced by R, D, C, E, Q, G, H, I, K, M, F, P, S, T, W, Y, V or L
YMAM

SEQ ID NO: 49 - protein, 4 aa, synthetic construct
  VARIANT 2: note = A can be replaced by R, N, D, C, E, Q, G, H, I, K, F, P, S, T, W, Y, V or L
  VARIANT 4: note = A can be replaced by R, N, D, C, E, Q, G, H, I, K, F, P, S, T, W, Y, V or L
YANA

SEQ ID NO: 50 - protein, 4 aa, synthetic construct
YENV

SEQ ID NO: 51 - protein, 4 aa, synthetic construct
YSNV

SEQ ID NO: 52 - protein, 4 aa, synthetic construct
YKNL

SEQ ID NO: 53 - protein, 4 aa, synthetic construct
YENQ

SEQ ID NO: 54 - protein, 4 aa, synthetic construct
YKNI

SEQ ID NO: 55 - protein, 4 aa, synthetic construct
YINQ

SEQ ID NO: 56 - protein, 4 aa, synthetic construct
YHNK

SEQ ID NO: 57 - protein, 4 aa, synthetic construct
YVNQ

SEQ ID NO: 58 - protein, 4 aa, synthetic construct
YLNP

SEQ ID NO: 59 - protein, 4 aa, synthetic construct
YLNT

SEQ ID NO: 60 - protein, 4 aa, synthetic construct
YDND

SEQ ID NO: 61 - protein, 4 aa, synthetic construct
YENI

SEQ ID NO: 62 - protein, 4 aa, synthetic construct
YENL

SEQ ID NO: 63 - protein, 4 aa, synthetic construct
YKNQ

SEQ ID NO: 64 - protein, 4 aa, synthetic construct
YKNV

SEQ ID NO: 65 - protein, 4 aa, synthetic construct
YANG

SEQ ID NO: 66 - protein, 4 aa, synthetic construct
YMDM

SEQ ID NO: 67 - protein, 4 aa, synthetic construct
YMPM

SEQ ID NO: 68 - protein, 4 aa, synthetic construct
YMRM

SEQ ID NO: 69 - protein, 4 aa, synthetic construct
YMSM

SEQ ID NO: 70 - protein, 4 aa, synthetic construct
  VARIANT 2: note = A can be replaced by R, N, C, E, Q, G, H, I, K, F, P, S, T, W, Y, V or L
  VARIANT 3: note = A can be replaced by R, D, C, E, Q, G, H, I, K, M, F, P, S, T, W, Y, V or L
YAAM

SEQ ID NO: 71 - protein, 4 aa, synthetic construct
YTHM

SEQ ID NO: 72 - protein, 4 aa, synthetic construct
YVLM

SEQ ID NO: 73 - protein, 4 aa, synthetic construct
YIAM

SEQ ID NO: 74 - protein, 4 aa, synthetic construct
YVEM

SEQ ID NO: 75 - protein, 4 aa, synthetic construct
YVKM

SEQ ID NO: 76 - protein, 4 aa, synthetic construct
YVPM

SEQ ID NO: 77 - protein, 4 aa, synthetic construct
  VARIANT 3: note = A can be replaced by R, D, C, E, Q, G, H, I, K, M, F, P, S, T, W, Y, V or L
  VARIANT 4: note = A can be replaced by R, N, C, E, Q, G, H, I, K, F, P, S, T, W, Y, V or L
YMAA

SEQ ID NO: 78 - protein, 4 aa, synthetic construct
YMAP

SEQ ID NO: 79 - protein, 4 aa, synthetic construct
  VARIANT 4: note = A can be replaced by R, C, E, Q, G, H, I, K, N, F, P, S, T, W, Y, V or L
YMNA

SEQ ID NO: 80 - protein, 4 aa, synthetic construct
  VARIANT 2: note = A can be replaced by R, C, E, Q, G, H, I, K, N, F, P, S, T, W, Y, V or L
YANM

SEQ ID NO: 81 - protein, 4 aa, synthetic construct
YMNV

SEQ ID NO: 82 - protein, 4 aa, synthetic construct
YENM

SEQ ID NO: 83 - protein, 4 aa, synthetic construct
YMNQ

SEQ ID NO: 84 - protein, 4 aa, synthetic construct
YMNL

SEQ ID NO: 85 - protein, 4 aa, synthetic construct
YSNM

SEQ ID NO: 86 - protein, 4 aa, synthetic construct
  VARIANT 3: note = A can be replaced by R, D, C, E, Q, G, H, I, K, M, F, P, S, T, W, Y, V or L
  VARIANT 2: note = A can be replaced by R, N, D, C, E, Q, G, H, I, K, F, P, S, T, W, Y, V or L
  VARIANT 4: note = A can be replaced by R, N, D, C, E, Q, G, H, I, K, F, P, S, T, W, Y, V or L
YAAA

SEQ ID NO: 87 - protein, 4 aa, synthetic construct
YGGG

SEQ ID NO: 88 - protein, 4 aa, synthetic construct
YAAA

SEQ ID NO: 89 - protein, 4 aa, synthetic construct
YFFF

SEQ ID NO: 90 - protein, 4 aa, synthetic construct
YETV

SEQ ID NO: 91 - protein, 4 aa, synthetic construct
YQQQ

SEQ ID NO: 92 - protein, 4 aa, synthetic construct
YHAE

SEQ ID NO: 93 - protein, 4 aa, synthetic construct
YLDL

SEQ ID NO: 94 - protein, 4 aa, synthetic construct
YLIP

SEQ ID NO: 95 - protein, 4 aa, synthetic construct
YLRV

SEQ ID NO: 96 - protein, 4 aa, synthetic construct
YTAV

SEQ ID NO: 97 - protein, 4 aa, synthetic construct
YVHV

SEQ ID NO: 98 - protein, 41 aa, synthetic construct
RSKRSRLLHSDYENVTPRRPGPTRKHYQPYAPPRDFAAYRS

SEQ ID NO: 99 - protein, 41 aa, synthetic construct
RSKRSRLLHSDYKNITPRRPGPTRKHYQPYAPPRDFAAYRS

SEQ ID NO: 100 - protein, 41 aa, synthetic construct
RSKRSRLLHSDYMDMTPRRPGPTRKHYQPYAPPRDFAAYRS

SEQ ID NO: 101 - protein, 41 aa, synthetic construct
RSKRSRLLHSDYGGGTPRRPGPTRKHYQPYAPPRDFAAYRS

SEQ ID NO: 102 - protein, 41 aa, synthetic construct
RSKRSRLLHSDYSNVTPRRPGPTRKHYQPYAPPRDFAAYRS

SEQ ID NO: 103 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 104 - protein, 261 aa, Mus musculus
MFQNAHSGSQWLLPPLTILLLFAFADRQSAALPKAVVKLDPPWIQVLKEDMVTLMCEGTHNPGNSSTQWFHNGRSIRSQVQASYTFKATVNDSGEYRCQMEQTRLSDPVDLGVISDWLLLQTPQRVFLEGETITLRCHSWRNKLLNRISFFHNEKSVRYHHYKSNFSIPKANHSHSGDYYCKGSLGSTQHQSKPVTITVQDPATTSSISLVWYHTAFSLVMCLLFAVDTGLYFYVRRNLQTPREYWRKSLSIRKHQAPQDK

SEQ ID NO: 105 - protein, 93 aa, Homo sapiens
MIHLGHILFLLLLPVAAAQTTPGERSSLPAFYPGTSGSCSGCGSLSLPLLAGLVAADAVASLLIVGAVFLCARPRRSPAQEDGKVYINMPGRG

SEQ ID NO: 106 - protein, 79 aa, Mus musculus
MDPPGYLLFLLLLPVAASQTSAGSCSGCGTLSLPLLAGLVAADAVMSLLIVGVVFVCMRPHGRPAQEDGRVYINMPGRG

SEQ ID NO: 107 - protein, 113 aa, Homo sapiens
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 108 - protein, 114 aa, Mus musculus
MGALEPSWCLLFLPVLLTVGGLSPVQAQSDTFPRCDCSSVSPGVLAGIVLGDLVLTLLIALAVYSLGRLVSRGQGTAEGTRKQHIAETESPYQELQGQRPEVYSDLNTQRQYYR

SEQ ID NO: 109 - protein, 370 aa, Homo sapiens
MLGQVVTLILLLLLKVYQGKGCQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 110 - protein, 397 aa, Mus musculus
MLGQAVLFTTFLLLRAHQGQDCPDSSEEVVGVSGKPVQLRPSNIQTKDVSVQWKKTEQGSHRKIEILNWYNDGPSWSNVSFSDIYGFDYGDFALSIKSAKLQDSGHYLLEITNTGGKVCNKNFQLLILDHVETPNLKAQWKPWTNGTCQLFLSCLVTKDDNVSYALYRGSTLISNQRNSTHWENQIDASSLHTYTCNVSNRASWANHTLNFTHGCQSVPSNFRFLPFGVIIVILVTLFLGAIICFCVWTKKRKQLQFSPKEPLTIYEYVKDSRASRDQQGCSRASGSPSAVQEDGRGQRELDRRVSEVLEQLPQQTFPGDRGTMYSMIQCKPSDSTSQEKCTVYSVVQPSRKSGSKKRNQNSSLSCTVYEEVGNPWLKAHNPARLSRRELENFDVYS

SEQ ID NO: 111 - protein, 336 aa, Homo sapiens
MDYPTLLLALLHVYRALCEEVLWHTSVPFAENMSLECVYPSMGILTQVEWFKIGTQQDSIAIFSPTHGMVIRKPYAERVYFLNSTMASNNMTLFFRNASEDDVGYYSCSLYTYPQGTWQKVIQVVQSDSFEAAVPSNSHIVSEPGKNVTLTCQPQMTWPVQAVRWEKIQPRQIDLLTYCNLVHGRNFTSKFPRQIVSNCSHGRWSVIVIPDVTVSDSGLYRCYLQASAGENETFVMRLTVAEGKTDNQYTLFVAGGTVLLLLFVISITTIIVIFLNRRRRRERRDLFTESWDTQKAPNNYRSPISTSQPTNQSMDDTREDIYVNYPTFSRRPKTRV

SEQ ID NO: 112 - protein, 333 aa, Mus musculus
MAYVTWLLAILHVHKALCEETLWDTTVRLSETMTLECVYPLTHNLTQVEWTKNTGTKTVSIAVYNPNHNMHIESNYLHRVHFLNSTVGFRNMSLSFYNASEADIGIYSCLFHAFPNGPWEKKIKVVWSDSFEIAAPSDSYLSAEPGQDVTLTCQLPRTWPVQQVIWEKVQPHQVDILASCNLSQETRYTSKYLRQTRSNCSQGSMKSILIIPNAMAADSGLYRCRSEAITGKNKSFVIRLIITDGGTNKHFILPIVGGLVSLLLVILIIIIFILYNRKRRRQVRIPLKEPRDKQSKVATNCRSPTSPIQSTDDEKEDIYVNYPTFSRRPKPRL

SEQ ID NO: 113 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 114 - protein, 133 aa, Homo sapiens
GIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 115 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 116 - protein, 270 aa, Homo sapiens
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 117 - protein, 176 aa, Homo sapiens
AFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 118 - protein, 172 aa, Homo sapiens
SGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 119 - protein, 162 aa, Homo sapiens
HDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 120 - protein, 159 aa, Homo sapiens
SITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 121 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 122 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 123 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 124 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 125 - protein, 556 aa, Homo sapiens
MGFWILAILTILMYSTAAKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKNPIDHHSIYCIIAVCSVFLMLINVLVIILKMFWIEATLLWRDIAKPYKTRNDGKLYDAYVVYPRNYKSSTDGASRVEHFVHQILPDVLENKCGYTLCIYGRDMLPGEDVVTAVETNIRKSRRHIFILTPQITHNKEFAYEQEVALHCALIQNDAKVILIEMEALSELDMLQAEALQDSLQHLMKVQGTIKWREDHIANKRSLNSKFWKHVRYQMPVPSKIPRKASSLTPLAAQKQ

SEQ ID NO: 126 - protein, 541 aa, Homo sapiens
MNCRELPLTLWVLISVSTAESCTSRPHITVVEGEPFYLKHCSCSLAHEIETTTKSWYKSSGSQEHVELNPRSSSRIALHDCVLEFWPVELNDTGSYFFQMKNYTQKWKLNVIRRNKHSCFTERQVTSKIVEVKKFFQITCENSYYQTLVNSTSLYKNCKKLLLENNKNPTIKKNAEFEDQGYYSCVHFLHHNGKLFNITKTFNITIVEDRSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKADMADIPGHVFTRGMIIAVLILVAVVCLVTVCVIYRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMPAKTVKPGRDEPEVLPVLSES

SEQ ID NO: 127 - protein, 662 aa, Homo sapiens
MEPLVTWVVPLLFLFLLSRQGAACRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 128 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 129 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 130 - protein, 153 aa, Homo sapiens [sequence omitted]

SEQ ID NO: 131 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 132 - protein, 144 aa, Homo sapiens
MLLAMVLTSALLLCSVAGQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 133 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 134 - protein, 825 aa, Homo sapiens
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLLLGVSVSCIVILAVCLLCYVSITKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 135 - protein, 459 aa, Homo sapiens
MTILGTTFGMVFSLLQVVSGESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 136 - protein, 521 aa, Homo sapiens
MGLGRCIWEGWTLESEALRRDMGTWLLACICICTCVCLGVSVTGEGQGPRSRTFTCLTNNILRIDCHWSAPELGQGSSPWLLFTSNQAPGGTHKCILRGSECTVVLPPEAVLVPSDNFTITFHHCMSGREQVSLVDPEYLPRRHVKLDPPSDLQSNISSGHCILTWSISPALEPMTTLLSYELAFKKQEEAWEQAQHRDHIVGVTWLILEAFELDPGFIHEARLRVQMATLEDDVVEEERYTGQWSEWSQPVCFQAPQRQGPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 137 - protein, 538 aa, Homo sapiens
MPRGWAAPLLLLLLQGGWGCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 138 - DNA, 20 bp, synthetic construct
  source 1..20: mol_type = other DNA
ggccgggttctagagtgcca

SEQ ID NO: 139 - protein, 4 aa, synthetic construct
PRRP

SEQ ID NO: 140 - protein, 4 aa, synthetic construct
PYAP

SEQ ID NO: 141 - protein, 18 aa, synthetic construct
GSTGSGSKPGSGEGSTKG

(19) United States
(12) Patent Application Publication
(10) Pub. No.: US 2026/0275289 A1
(43) Pub. Date: Sep. 17, 2026

(54) CHIMERIC RECEPTORS AND USES THEREOF

(71) Applicants: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(72) Inventors: Sanam Shahid, New York, NY (US); Anthony Daniyan, Somers, NY (US); Katharine C. Hsu, New York, NY (US)

(73) Assignees: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(21) Appl. No.: 19/649,953

(22) Filed: Apr. 16, 2026

Related U.S. Application Data
(63) Continuation of application No. PCT/US2024/051928, filed on Oct. 18, 2024.
(60) Provisional application No. 63/591,222, filed on Oct. 18, 2023.
(60) Provisional application No. 63/551,316, filed on Feb. 8, 2024.

(51) Int. Cl. C12N 5/0783; A61K 40/15; A61K 40/31; A61K 40/42; A61P 35/02; C07K 14/54; C07K 14/705; C07K 16/28

(52) U.S. Cl. CPC C12N 5/0646; A61K 40/15; A61K 40/31; A61K 40/421; A61P 35/02; C07K 14/5443; C07K 14/7051; C07K 14/70521; C07K 16/2803; A61K 2239/13; A61K 2239/21; A61K 2239/22; A61K 2239/48; C07K 2317/73; C07K 2317/92; C07K 2319/03; C12N 2510/00

(57) ABSTRACT

The presently disclosed subject matter provides methods and compositions for enhancing immune responses toward tumor and pathogen antigens. It relates to chimeric receptors that can be expressed in cells (e.g., NK cells) to improve the activity of the cells.

### CROSS-REFERENCE TO RELATED APPLICATIONS

This application is a continuation application of International Patent Application No. PCT/US2024/051928, filed on Oct. 18, 2024, which claims priority to U.S. Provisional Application No. 63/591,222, filed Oct. 18, 2023, and to U.S. Provisional Application No. 63/551,316, filed Feb. 8, 2024, the contents of each of which are incorporated by reference in their entireties, and to each of which priority is claimed.

### SEQUENCE LISTING

The instant application contains a Sequence Listing which has been submitted via Patent Center and is hereby incorporated by reference in its entirety. Said.xml copy, created on Apr. 16, 2026, is named 0727341916, and is 141,565 bytes in size.

### INTRODUCTION

The presently disclosed subject matter provides chimeric receptors that can improve the activity of immunotherapy.

### BACKGROUND OF THE INVENTION

Cell-based immunotherapy has proven curative potential for the treatment of cancer. For example, NK cells and other immunoresponsive cells can be modified to target tumor antigens through the introduction of genetic material coding for chimeric receptors capable of specifically binding to selected antigens. Such engineered immunoresponsive cells comprising chimeric receptors have demonstrated remarkable efficacy against a range of malignancies. Treatment failures and relapses, however, occur in a significant fraction of patients. Therefore, there remain needs in the art for immunotherapies exhibiting improved activity.

### SUMMARY OF THE INVENTION

The presently disclosed subject matter provides chimeric receptors that can improve the activity of immunotherapy. In certain non-limiting embodiments, the presently disclosed subject matter provides immunoresponsive cells comprising a chimeric receptor that binds to an antigen without delivering (1) an activation signal; and/or (2) a co-stimulatory signal to the immunoresponsive cell. In certain embodiments, the chimeric receptor comprises an extracellular antigen-binding domain and a transmembrane domain. In certain embodiments, the chimeric receptor comprises an intracellular polypeptide.

In certain embodiments, the transmembrane domain comprises a CD8 polypeptide, a CD28 polypeptide, a CD3 (polypeptide, a CD4 polypeptide, a 4-1BB polypeptide, an OX40 polypeptide, an ICOS polypeptide, a CD84 polypeptide, a CD166 polypeptide, a CD8a polypeptide, a CD8b polypeptide, a ICAM-1 polypeptide, a CTLA-4 polypeptide, a CD27 polypeptide, a CD40 polypeptide, a NKGD2 polypeptide, a PD-1 polypeptide, a LAG-3 polypeptide, a 2B4 polypeptide, a BTLA polypeptide, a DAP12 polypeptide, A DAP10 polypeptide, a CD16 polypeptide, a DNAM1 polypeptide, or a combination thereof.

In certain embodiments, the intracellular polypeptide comprises a CD8 polypeptide, a CD28 polypeptide, a CD4 polypeptide, a 4-1BB polypeptide, an OX40 polypeptide, an ICOS polypeptide, a CD84 polypeptide, a CD166 polypeptide, a CD8a polypeptide, a CD8b polypeptide, a ICAM-1 polypeptide, a CTLA-4 polypeptide, a CD27 polypeptide, a CD40 polypeptide, a NKGD2 polypeptide, a 2B4 polypeptide, a DAP12 polypeptide, a DAP-10 polypeptide, a CD16 polypeptide, a DNAM1 polypeptide, or a combination thereof.

In certain embodiments, the intracellular polypeptide comprises a CD28 polypeptide. In certain embodiments, the intracellular polypeptide comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% identical to the amino acid sequence set forth in SEQ ID NO: 16. In certain embodiments, the intracellular polypeptide comprises the amino acid sequence set forth in SEQ ID NO: 16. In certain embodiments, the intracellular polypeptide consists of the amino acid sequence set forth in SEQ ID NO: 16.

In certain embodiments, the intracellular polypeptide lacks a CD3ζ polypeptide.

In certain embodiments, the antigen is a tumor antigen or a pathogen antigen. In certain embodiments, the antigen is a tumor antigen. In certain embodiments, the tumor antigen is selected from CD33, CD19, carbonic anhydrase IX (CAIX), carcinoembryonic antigen (CEA), CD8, CD7, CD10, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinases Erb-B2,3,4 (erb-B2,3,4), folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Ra2), K-light chain, kinase insert domain receptor (KDR), Lewis Y (LeY), L1 cell adhesion molecule (LICAM), melanoma antigen family A, 1 (MAGE-A1), Mucin 16 (MUC16), Mucin 1 (MUC1), Mesothelin (MSLN), ERBB2, MAGEA3, p53, MART1,GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, NKCS1, EGFIR, EGFR-VIII, CD99, CD70, ADGRE2, CCR1, LILRB2, PRAME CCR4, CD5, CD3, TRBC1, TRBC2, TIM-3, Integrin B7, ICAM-1, CD70, Tim3, CLEC12A, or ERBB. In certain embodiments, the antigen is CD33. In certain embodiments, the extracellular antigen-binding domain comprises a heavy chain variable region (VH) comprising a CDR1 comprising the amino acid sequence set forth in SEQ ID NO: 28, a CDR2 comprising the amino acid sequence set forth in SEQ ID NO: 29, and a CDR3 comprising the amino acid sequence set forth in SEQ ID NO: 30, and a light chain variable region (VL) comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33.

In certain embodiments, the chimeric receptor comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% identical to the amino acid sequence set forth in SEQ ID NO: 21 or SEQ ID NO: 22. In certain embodiments, the chimeric receptor comprises the amino acid sequence set forth in SEQ ID NO: 21 or SEQ ID NO: 22. In certain embodiments, the chimeric receptor is recombinantly expressed. In certain embodiments, the chimeric receptor is expressed from a vector.

In certain embodiments, the immunoresponsive cell is a cell of the lymphoid lineage or a cell of the myeloid lineage. In certain embodiments, the cell of the lymphoid lineage is selected from T cells, B cells, Natural Killer (NK) cells, or dendritic cells. In certain embodiments, the immunoresponsive cell is a NK cell or a NKG2C⁺CD8⁺ T cell. In certain embodiments, the immunoresponsive cell is derived from an induced pluripotent stem cell. In certain embodiments, said cell is autologous or allogeneic.

In certain embodiments, the immunoresponsive cell further comprises a first cytokine selected from the group consisting of an IL-15 polypeptide or a functional fragment thereof, an IL-33 polypeptide or a functional fragment thereof, an IL-18 polypeptide or a functional fragment thereof, an IL-12 polypeptide or a functional fragment thereof, an IL-2 polypeptide or a functional fragment thereof, an IL-4 polypeptide or a functional fragment thereof, an IL-7 polypeptide or a functional fragment thereof, an IL-9 polypeptide or a functional fragment thereof, and an IL-21 polypeptide or a functional fragment thereof. In certain embodiments, the immunoresponsive cell further comprises a second cytokine selected from the group consisting of an IL-15 polypeptide or a functional fragment thereof, an IL-33 polypeptide or a functional fragment thereof, an IL-18 polypeptide or a functional fragment thereof, an IL-12 polypeptide or a functional fragment thereof, an IL-2 polypeptide or a functional fragment thereof, an IL-4 polypeptide or a functional fragment thereof, an IL-7 polypeptide or a functional fragment thereof, an IL-9 polypeptide or a functional fragment thereof, and an IL-21 polypeptide or a functional fragment thereof. In certain embodiments, the immunoresponsive cell further comprises a cytokine receptor selected from the group consisting of an IL-15 receptor polypeptide, a ST2 polypeptide, an IL-18 receptor polypeptide, an IL-12 receptor polypeptide, an IL-2 receptor polypeptide, an IL-4 receptor polypeptide, an IL-7 receptor polypeptide, an IL-9 receptor polypeptide, and an IL-21 receptor polypeptide.

In certain embodiments, the immunoresponsive cell further comprises an antigen-recognizing receptor that targets a second antigen. In certain embodiments, the second antigen-recognizing receptor is a chimeric antigen receptor (CAR), a T cell receptor (TCR), a chimeric ligand receptor, a chimeric co-stimulating receptor (CCR), or a combination thereof.

In certain non-limiting embodiments, the presently disclosed subject matter provides a nucleic acid encoding a chimeric receptor that binds to an antigen and that does not deliver an activation signal and a co-stimulatory signal to the immunoresponsive cell. In certain embodiments, the chimeric receptor comprises an extracellular antigen-binding domain and a transmembrane domain. In certain embodiments, the chimeric receptor comprises an intracellular polypeptide.

In certain embodiments, the transmembrane domain comprises a CD8 polypeptide, a CD28 polypeptide, a CD3 (polypeptide, a CD4 polypeptide, a 4-1BB polypeptide, an OX40 polypeptide, an ICOS polypeptide, a CD84 polypeptide, a CD166 polypeptide, a CD8a polypeptide, a CD8b polypeptide, a ICAM-1 polypeptide, a CTLA-4 polypeptide, a CD27 polypeptide, a CD40 polypeptide, a NKGD2 polypeptide, a PD-1 polypeptide, a LAG-3 polypeptide, a 2B4 polypeptide, a BTLA polypeptide, a DAP12 polypeptide, A DAP10 polypeptide, a CD16 polypeptide, a DNAM1 polypeptide, or a combination thereof.

In certain embodiments, the intracellular polypeptide comprises a CD8 polypeptide, a CD28 polypeptide, a CD4 polypeptide, a 4-1BB polypeptide, an OX40 polypeptide, an ICOS polypeptide, a CD84 polypeptide, a CD166 polypeptide, a CD8a polypeptide, a CD8b polypeptide, a ICAM-1 polypeptide, a CTLA-4 polypeptide, a CD27 polypeptide, a CD40 polypeptide, a NKGD2 polypeptide, a 2B4 polypeptide, a DAP12 polypeptide, a DAP-10 polypeptide, a CD16 polypeptide, a DNAM1 polypeptide, or a combination thereof.

In certain embodiments, the intracellular polypeptide comprises a CD28 polypeptide. In certain embodiments, the intracellular polypeptide lacks a CD32 polypeptide.

In certain embodiments, the nucleic acid further comprises a promoter that is operably linked to the antigen-recognizing receptor. In certain embodiments, said promoter is endogenous or exogenous. In certain embodiments, the exogenous promoter is selected from the group consisting of an elongation factor (EF)-1 promoter, a CMV promoter, a SV40 promoter, a PGK promoter, and a metallothionein promoter. In certain embodiments, said promoter is an inducible promoter. In certain embodiments, the inducible promoter is selected from the group consisting of a NFAT transcriptional response element (TRE) promoter, a CD69 promoter, a CD25 promoter, and an IL-2 promoter.

In certain non-limiting embodiments, the presently disclosed subject matter provides vectors and lipid nanoparticles comprising the nucleic acids disclosed herein. In certain non-limiting embodiments, the presently disclosed subject matter provides cells comprising the nucleic acids, the vectors, or the lipid nanoparticles disclosed herein.

In certain non-limiting embodiments, the presently disclosed subject matter provides compositions comprising the nucleic acids, the vectors, the lipid nanoparticles, or the cells disclosed herein. In certain embodiments, the compositions are pharmaceutical compositions further comprising pharmaceutically acceptable excipients.

In certain non-limiting embodiments, the presently disclosed subject matter provides a method of reducing tumor burden in a subject, the method comprising administering the nucleic acids, the vectors, the lipid nanoparticles, the cells, or the compositions disclosed herein. In certain embodiments, the method reduces the number of tumor cells, reduces tumor size, and/or eradicates the tumor in the subject.

In certain non-limiting embodiments, the presently disclosed subject matter provides a method of treating a subject having a relapse of a neoplasm, the method comprising administering the nucleic acids, the vectors, the lipid nanoparticles, the cells, or the compositions disclosed herein. In certain non-limiting embodiments, the presently disclosed subject matter provides a method of treating and/or preventing a neoplasm in a subject, the method comprising administering the nucleic acids, the vectors, the lipid nanoparticles, the cells, or the compositions disclosed herein.

In certain embodiments, the neoplasm or tumor is cancer. In certain embodiments, the neoplasm or tumor is selected from the group consisting of blood cancers and solid tumors. In certain embodiments, the blood cancer is multiple myeloma, myeloid disorder, leukemia, or lymphoma. In certain embodiments, the leukemia is acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, or B cell prolymphocytic leukemia. In certain embodiments, the leukemia is acute myeloid leukemia (AML).

In certain embodiments, the lymphoma is Hodgkin's lymphoma, non-Hodgkin's lymphoma, B-cell non-Hodgkin's lymphoma, or T-cell non-Hodgkin's lymphoma. In certain embodiments, the solid tumor is selected from the group consisting of renal cell carcinoma, non-small-cell lung cancer, lung adenocarcinoma, lung squamous cell carcinoma, lung neuroendocrine carcinoma, small-cell lung cancer, pancreatic cancer, breast cancer, astrocytoma, glioblastoma, laryngeal/pharyngeal carcinoma, EBV-associated nasopharyngeal carcinoma, and ovarian carcinoma. In certain embodiments, the subject is a human.

In certain non-limiting embodiments, the presently disclosed subject matter provides a method for producing a cell, the method comprising introducing into a cell the nucleic acids, the vectors, or the lipid nanoparticles disclosed herein.

In certain non-limiting embodiments, the presently disclosed subject matter provides 56.

A kit comprising the nucleic acids, the vectors, the lipid nanoparticles, the cells, or the compositions disclosed herein. In certain embodiments, the kit further comprises written instructions for treating and/or preventing a neoplasm, a pathogen infection, and/or an infectious disease.

### BRIEF DESCRIPTION OF THE FIGURES

The following Detailed Description includes exemplary descriptions of the subject matter of the present disclosure, but is not intended to limit the instant disclosure to the specific embodiments described and is further elaborated in conjunction with the accompanying drawings.

FIGS. 1A-1D illustrate the generation of CAR constructs and transduction of peripheral blood-derived NK cells. FIG. 1A shows mCherry-3p14mt28z and mCherry-3p14mtDEL CAR constructs. FIG. 1B shows NK cell transduction efficiency as measured by flow cytometry for mCherry. P-values determined by one-way ANOVA. ns, non-significant. FIGS. 1C and 1D show representative FACS plots for mCherry vs. (1C) IgG detected using goat anti-human IgG or (1D) myc tag positivity (Y axis) in NK cells transduced with the indicated constructs.

FIGS. 2A-2D illustrate the in vitro phenotyping and expansion of CAR construct-transduced and control NK cells. FIG. 1A shows fold expansion of NK cells after transduction. Data represent 3 technical replicates from one representative donor. P-values determined by unpaired t-test; ns, non-significant. FIGS. 2B and 2C show frequency of expression of NK cell receptors and immune checkpoints in transduced NK cells from one representative healthy donor (2B) prior to and 6 days after activation and (2C) over 28 days of expansion following activation with IL-2 and C9.mbIL21 cells. FIG. 2D shows OCIAML2 cell killing and CAR+NK cell expansion in a representative 14-day proliferation assay. Black arrows indicate when target cells (OCI-AML2) were added. All results are representative of assays performed in 3-4 donors.

FIGS. 3A-3F illustrate in vitro activation and cytotoxicity of mCherry-3p14mt28z and mCherry-3p14mtDEL. FIG. 3A shows CD107a degranulation measured by flow cytometry gated on CAR+ and CAR− fractions of NK cells from one representative donor transduced as indicated against 3 AML cell lines. FIG. 3B shows IFN-γ release measured by flow cytometry gated on CAR+ and CAR− fractions of NK cells from one representative donor transduced as indicated against 3 AML cell lines. FIGS. 3C and 3D show percent killing of indicated AML cell lines by NK cells from one representative donor transduced with the CAR or CAR-DEL construct (3C) at various E:T ratios after 24 h of co-culture and (3D) at an E:T ratio of 1:2 after 1, 2, or 3 target challenges (added every 24 h). P-values determined by multiple unpaired t tests. FIG. 3E shows percent killing of control vs. CD33-knockout OCI-AML2-gfpLuc+ cells by NK cells from one representative donor transduced with the CAR or CAR-DEL construct at an E:T ratio of 1:1 after 24 h of co-culture. P-values determined by multiple unpaired t tests. Data represent 3 technical replicates. FIG. 3F shows proliferation of HL60-gfpLuc+ (E:T 1:1) and OCI-AML2-gfpLuc+ (E:T 1:4) (top) and NK cells from one representative donor transduced with the CAR or CAR-DEL construct over 160 h of co-culture. All results are representative of assays performed in 3-4 donors. *p<0.05, **p<0.01, and ***p<0.001.

FIGS. 4A-4E illustrate in vitro activation and cytotoxicity of cytokine-armored CD33-targeted CAR-modified NK cells. FIG. 4A shows cytokine-armored CAR constructs generated and evaluated in this study. FIGS. 4B and 4C show the percent killing of indicated AML cell lines by transduced NK cells from one representative donor transduced with control or armored CD33-targeted CAR constructs (4B) at various E:T ratios after 24 h of co-culture and (4C) at an E:T ratio of 1:2 after 1, 2, or 3 target challenges (added every 24 h). FIGS. 4D and 4E show the proliferation of OCIAML2 cells and CAR+ NK cells from a representative donor transduced with control or armored CAR or CAR-DEL constructs over (4D) 22 days, with additional target cells added during co-culture (black arrows), and (4E) over 160 h. All results are representative of assays performed in 3-4 donors.

FIGS. 5A-5D illustrate in vivo antitumor efficacy of CD33-targeted CAR-modified NK cells. FIGS. 5A and 5B show CAR-modified and control NK cells' (4 injections on indicated days) effects on (5A) tumor growth as measured by bioluminescence (p/s) measured weekly and (5B) survival of OCI-AML2-engrafted NCG mice. n=5 mice per group. FIGS. 5C and 5D show the cytokine-armored CAR-modified and control NK cells' effects on (5C) tumor growth as measured by bioluminescence (p/s) measured at day 13 (after 2 NK cell injections and (5D) survival of OCI-AML2-engrafted NCG mice. P values were determined by Mann-Whitney tests for tumor growth and by log-rank test for survival. n=5-13 mice in C and 5 mice in D per treatment group. *p<0.05, **p<0.01, and ***p<0.001.

FIGS. 6A-6C illustrate the toxicity associated with IL15-armored CAR-modified NK cells in vivo. In OCI-AML2-engrafted NCG mice treated with the indicated NK cells with or without exogenous IL-15. FIG. 6A shows representative hematoxylin and eosin (H&E) staining (left) and immunohistochemistry for GFP and mCherry of lung tissue. FIG. 6B shows hematology findings. FIG. 6C shows serum cytokine levels. P-values determined by multiple unpaired t-tests. Data represent 1-3 biological replicates. *p<0.05, **p<0.01, and ***p<0.001.

FIGS. 7A-7C depict the evaluation of NK cell expansion and transduction protocols. FIG. 7A shows the schema showing workflow of NK cell isolation, expansion, and transduction. FIG. 7B shows the fold expansion of NK cells over 6 days prior to transduction. Data represent replicates from 4 unique donors. FIG. 7C shows the percent killing of OCIAML2 by NK cells from one representative donor transduced with the CAR construct and freshly expanded for 16, 23, or 30 days as well as frozen after 16 days of expansion.

FIGS. 8A-8D illustrate a comparison of different K562-based feeder cells. FIG. 8A shows the fold expansion of NK cells over 6 days prior to transduction with 4 different feeder cells from one representative healthy donor. FIG. 8B shows the fold expansion of transduced NK cells (co-cultured with 4 different feeder cells pre-transduction) over 15 days after transduction from one representative healthy donor. FIG. 8C shows the percent killing of OCIAML2 by NK cells from one representative donor transduced with the CAR construct (top), mock CAR (middle), and non-transduced (bottom). FIG. 8D shows the frequency of expression of immune checkpoints in transduced NK cells expanded with 4 different feeder cells pre-transduction from one representative healthy donor 16 days after post-transduction expansion.

FIG. 9 illustrates K562-based feeder cell phenotyping. Expression of 41BBL, mbIL21, and CD33 as measured by flow cytometry of parental K562s (left) and feeder cells (right).

FIGS. 10A and 10B illustrate AML cell line phenotyping. FIG. 10A shows the expression of CD33, HLA-A/B/C, HLA-E, and CD19 as measured by flow cytometry of HL-60, OCI-AML2, and OCI-AML3. FIG. 10B shows the expression of CD33 and GFP as measured by flow cytometry of OCI-AML2-wild-type and CD33 knock-outs.

FIG. 11 illustrates phenotyping of CAR-modified NK cells with cytokine transgenes. Frequency of expression of NK cell receptors and immune checkpoints in transduced NK cells from one representative healthy donor 6 days after activation with CD33+ targets.

FIGS. 12A-12I illustrate in vivo model development. FIG. 12A shows a schema depicting the evaluation of the in vivo model. FIG. 12B shows the survival of OCI-AML2-engrafted NCG mice without any treatment. FIG. 12C shows the survival of OCI-AML2-engrafted NCG mice treated with one dose of NK cells given at day +7 at various doses. FIG. 12D shows the survival of OCI-AML2-engrafted NCG mice treated with one dose of NK cells given at day +7 and exogenous IL15 twice weekly. FIG. 12E shows the survival of OCI-AML2-engrafted NCG mice treated with two doses of NK cells given at day +3 and day +10 and exogenous IL15 twice weekly. FIG. 12F shows the survival of 250,000 OCI-AML2-engrafted NCG mice treated with four doses of NK cells and exogenous IL15 twice weekly. FIG. 12G shows the survival of 50,000 OCI-AML2-engrafted NCG mice treated with four doses of NK cells and exogenous IL15 twice weekly. FIG. 12H shows bioluminescence measured at day +20 in OCI-AML2 engrafted NSGS and NCG mice treated with four doses of NK cells and exogenous IL15 twice weekly. FIG. 12I shows the survival of OCI-AML2-engrafted NCG mice treated with four doses of NK cells and exogenous IL15 given in non-cytokine secreting NK cell groups. P values were determined by Mann-Whitney tests for tumor growth and by log-rank test for survival. n=5 mice per treatment group. *p<0.05, **p<0.01, and ***p<0.001.

FIG. 13 illustrates in vitro cytotoxicity based on varying NK cell education. Percent killing of indicated AML cell lines by NK cells from 3 donors with varying NK cell education levels transduced with the CAR or CAR-DEL construct at various E:T ratios after 24 h of co-culture.

### DETAILED DESCRIPTION OF THE INVENTION

Malignant cells have developed a series of mechanisms to protect themselves from immune recognition and elimination. The presently disclosed subject matter provides chimeric receptors that are capable of enhancing the activity of immunotherapy (e.g., NK cell immunotherapy) by, in certain embodiments, overcoming such hostile tumor microenvironments. The chimeric receptors of the presently disclosed subject matter can enhance the activity of cells (e.g., NK cells). The presently disclosed subject matter also provides methods of using such chimeric receptors for inducing and/or enhancing an immune response of a cell to a target antigen, and/or treating and/or preventing neoplasms or other diseases/disorders (e.g., autoimmune diseases and infectious diseases), e.g., where an increase in an antigen-specific immune response is desired. The presently disclosed subject matter is based, at least in part, on the unexpected discovery that a chimeric receptor lacking certain intracellular domains disclosed herein can enhance the activity (e.g., cytotoxicity) of an immunoresponsive cell (e.g., NK cell).

Non-limiting embodiments of the present disclosure are described by the present specification and Examples. For purposes of clarity of disclosure and not by way of limitation, the detailed description is divided into the following subsections:
1. Definitions;
2. Cells;
3. Nucleic Acids and Compositions;
4. Polypeptides;
5. Formulations and Administration;
6 Methods of Treatment;
7. Kits; and
8. Exemplary Embodiments.

### 1. Definitions

Unless defined otherwise, all technical and scientific terms used herein have the meaning commonly understood by a person skilled in the art. The following references provide one of skill with a general definition of many of the terms used in the presently disclosed subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold or within 2-fold, of a value.

“Antibody” and “antibodies” as those terms are known in the art refer to antigen binding proteins of the immune system. The term “antibody” as referred to herein includes whole, full length antibodies having an antigen-binding region, and any fragment thereof in which the “antigen-binding fragment” or “antigen-binding region” is retained, or single chains, for example, single chain variable fragment (scFv), thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant (CH) region. The heavy chain constant region is comprised of three domains, CH1, CH2 and CH3. Each light chain is comprised of a light chain variable region (abbreviated herein as VL) and a light chain constant CL region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding fragment” or “antigen-binding region” of an antibody, as used herein, refers to that region or fragment of the antibody that binds to the antigen and which confers antigen specificity to the antibody; fragments of antigen-binding proteins, for example, antibodies include one or more fragments of an antibody that retain the ability to specifically bind to an antigen (e.g., a CD3 polypeptide). It has been shown that the antigen-binding function of an antibody can be performed by fragments of a full-length antibody. Examples of antigen-binding fragments encompassed within the term “antibody fragments” of an antibody include a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., Nature 1989; 341:544-546), which consists of a VH domain; and an isolated complementarity determining region (CDR).

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules. These are known as single chain Fv (scFv); see e.g., Bird et al., Science (1988); 242:423-426; and Huston et al., Proc Natl Acad Sci (1998); 85:5879-5883. These antibody fragments are obtained using conventional techniques known to those of skill in the art, and the fragments are screened for utility in the same manner as are intact antibodies.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the presently disclosed subject matter may include amino acid residues not encoded by human germline immunoglobulin sequences (e.g., mutations introduced by random or site-specific mutagenesis in vitro or by somatic mutation in vivo).

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring the production of the antibody by any particular method. For example, the monoclonal antibodies to be used in accordance with the presently disclosed subject matter may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “recombinant human antibody”, as used herein, includes all human antibodies that are prepared, expressed, created, or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, the term “specifically binds to,” when referring to an antibody or an antigen-binding fragment thereof, means that the antibody or antigen-binding fragment thereof binds to a desired target (e.g., human CD33) with a dissociation constant (KD) of about 1× 10⁻⁸ M or less, about 5×10⁻⁹ M or less, about 1× 10⁻⁹ M or less, about 5×10⁻¹⁰ M or less, about 1×10⁻¹⁰ M or less, about 5×10⁻¹¹ M or less, or about 1×10⁻¹¹ M or less.

An “antibody that competes for binding” or “antibody that cross-competes for binding” with a reference antibody for binding to an antigen, e.g., CD33, refers to an antibody that blocks binding of the reference antibody to the antigen (e.g., CD33) in a competition assay by 50% or more, and conversely, the reference antibody blocks binding of the antibody to the antigen (e.g., CD33) in a competition assay by 50% or more. An exemplary competition assay is described in “Antibodies”, Harlow and Lane (Cold Spring Harbor Press, Cold Spring Harbor, NY).

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen (e.g., a CD33 polypeptide).”

As used herein, the term “single-chain variable fragment” or “scFv” is a fusion protein of the variable regions of the heavy (VH) and light chains (VL) of an immunoglobulin (e.g., mouse or human) covalently linked to form a VH::VL heterodimer. The heavy (VH) and light chains (VL) are either joined directly or joined by a peptide-encoding linker (e.g., 10, 15, 20, 25 amino acids), which connects the N-terminus of the VH with the C terminus of the VL, or the C-terminus of the VH with the N-terminus of the VL. The linker is usually rich in glycine for flexibility, as well as serine or threonine for solubility. The linker can link the heavy chain variable region and the light chain variable region of the extracellular antigen-binding domain.

Non-limiting examples of linkers, e.g., for use in generating an scFv, are disclosed in Shen et al., Anal Chem (2008); 80 (6): 1910-1917 and WO 2014/087010, the contents of which are hereby incorporated by reference in their entireties. In certain embodiments, the linker is a G4S linker. In certain embodiments, the linker comprises or consists of the amino acid sequence set forth in SEQ ID NO: 1, which is provided below:

[SEQ ID NO: 1]
GGGGSGGGGSGGGSGGGGS

In certain embodiments, the linker comprises or consists of the amino acid sequence set forth in SEQ ID NO: 2, which is provided below:
GGGGSGGGGSGGGGS [SEQ ID NO: 2]

In certain embodiments, the linker comprises or consists of the amino acid sequence set forth in SEQ ID NO: 3, which is provided below:

[SEQ ID NO: 3]
GGGGSGGGGSGGGGSGGGSGGGGS

In certain embodiments, the linker comprises or consists of the amino acid sequence set forth in SEQ ID NO: 4, which is provided below:

[SEQ ID NO: 4]
GGGGSGGGGSGGGGSGGGGSGGGSGGGGS

In certain embodiments, the linker comprises or consists of the amino acid sequence set forth in SEQ ID NO: 5, which is provided below:

[SEQ ID NO: 5]
GGGGS

In certain embodiments, the linker comprises or consists of the amino acid sequence set forth in SEQ ID NO: 6, which is provided below:

[SEQ ID NO: 6]
GGGGSGGGGS

In certain embodiments, the linker comprises or consists of the amino acid sequence set forth in SEQ ID NO: 7, which is provided below:

[SEQ ID NO: 7]
GGGGSGGGGSGGGGSGGGGS

Additionally or alternately, the linker can be a Whitlow/218 linker disclosed in Whitlow, M. et al. (1993) Protein Eng 6, 989-95, the contents of which are hereby incorporated by reference in their entireties. In certain embodiments, the linker comprises or consists of the amino acid sequence set forth in SEQ ID NO: 141, which is provided below:

SEQ ID NO: 141]
GSTGSGSKPGSGEGSTKG

Despite removal of the constant regions and the introduction of a linker, scFv proteins retain the specificity of the original immunoglobulin. Single chain Fv polypeptide antibodies can be expressed from a nucleic acid comprising VH- and VL-encoding sequences as described by Huston, et al. (Proc. Nat. Acad. Sci. USA, 1988; 85:5879-5883). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hybridoma (Larchmt) 2008; 27 (6): 455-51; Peter et al., J Cachexia Sarcopenia Muscle 2012 Aug. 12; Shieh et al., J Imunol 2009; 183 (4): 2277-85; Giomarelli et al., Thromb Haemost 2007; 97 (6): 955-63; Fife eta., J Clin Invst 2006; 116 (8): 2252-61; Brocks et al., Immunotechnology 1997; 3 (3): 173-84; Moosmayer et al., Ther Immunol 1995; 2 (10:31-40). Agonistic scFvs having stimulatory activity have been described (see, e.g., Peter et al., J Bio. Chem 2003; 25278 (38): 36740-7; Xie et al., Nat Biotech 1997; 15 (8): 768-71; Ledbetter et al., Crit Rev Immunol 1997; 17 (5-6): 427-55; Ho et al., BioChim Biophys Acta 2003; 1638 (3): 257-66).

As used herein, “F(ab)” refers to a fragment of an antibody structure that binds to an antigen but is monovalent and does not have a Fc portion, for example, an antibody digested by the enzyme papain yields two F(ab) fragments and an Fc fragment (e.g., a heavy (H) chain constant region; Fc region that does not bind to an antigen).

As used herein, “F(ab′)₂” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)₂” fragment can be split into two individual Fab′ fragments.

As used herein, the term “nanobody” or “single-domain antibody” refers to small antigen-binding fragments that are derived from heavy chain only antibodies present in camelids (VHH, from camels and llamas), and cartilaginous fishes (VNAR, from sharks). Nanobodies are useful alternatives to conventional antibodies due to their small size, and high solubility and stability.

As used herein, the term “vector” refers to any genetic element, such as a plasmid, phage, transposon, cosmid, chromosome, virus, virion, etc., which is capable of replication when associated with the proper control elements and which can transfer gene sequences into cells. Thus, the term includes cloning and expression vehicles, as well as viral vectors and plasmid vectors.

“CDRs” are defined as the complementarity determining region amino acid sequences of an antibody which are the hypervariable regions of immunoglobulin heavy and light chains. See, e. g., Kabat et al., Sequences of Proteins of Immunological Interest, 4th U. S. Department of Health and Human Services, National Institutes of Health (1987), or IMGT numbering system (Lefranc, The Immunologist (1999); 7:132-136; Lefranc et al., Dev. Comp. Immunol. (2003); 27:55-77). The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise three heavy chain and three light chain CDRs or CDR regions in the variable region. CDRs provide the majority of contact residues for the binding of the antibody to the antigen or epitope region. In certain embodiments, the CDRs are identified according to the IMGT system. In certain embodiments, the CDRs are identified using the IMGT numbering system accessible at http://www.imgt.org/IMGT_vquest/input.

As used herein, a “co-stimulatory molecule polypeptide” refers to a polypeptide of a cell surface molecule other than an antigen receptor or its ligand that can provide an efficient response of lymphocytes to an antigen. In certain embodiments, a co-stimulatory molecule polypeptide can provide activation of cells of the immune system.

As used herein, a “co-stimulatory ligand polypeptide” refers to a polypeptide of a molecule that upon binding to its receptor (e.g., a co-stimulatory molecule) produces a co-stimulatory response, e.g., an intracellular response that effects the stimulation provided when an antigen-recognizing receptor (e.g., a chimeric antigen receptor (CAR)) binds to its target antigen.

As used herein, “a functional fragment” of a molecule or polypeptide includes a fragment of the molecule or polypeptide that retains at least about 80%, at least about 85%, at least about 90%, at least about 95%, or at least about 100% of the primary function of the molecule or polypeptide.

The term “antigen-recognizing receptor” as used herein refers to a receptor that is capable of activating an immune or immunoresponsive cell (e.g., a NK cell) in response to its binding to an antigen.

The term “chimeric antigen receptor” or “CAR” as used herein refers to a molecule comprising an extracellular antigen-binding domain that is fused to an intracellular signaling domain that is capable of activating or stimulating an immune or immunoresponsive cell, and a transmembrane domain. In certain embodiments, the extracellular antigen-binding domain of a CAR comprises a scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain. In certain embodiments, the CAR is selected to have high binding affinity or avidity for the antigen.

The term “antigen-binding domain” as used herein refers to a domain capable of specifically binding a particular antigenic determinant or set of antigenic determinants present on a cell.

By “receptor” is meant a polypeptide, or portion thereof, present on a cell membrane that selectively binds one or more ligands.

By “recognize” is meant selectively binds to a target. An immunoresponsive cell (e.g., NK cell, T cell) that recognizes a tumor can express a receptor (e.g., CAR-DEL, TCR, or CAR) that binds to a tumor antigen.

By “immunoresponsive cell” is meant a cell that functions in an immune response or a progenitor, or progeny thereof. In certain embodiments, the immunoresponsive cell is a cell of lymphoid lineage. Non-limiting examples of cells of lymphoid lineage include T cells, Natural Killer (NK) cells, B cells, and stem cells from which lymphoid cells may be differentiated. In certain embodiments, the immunoresponsive cell is a cell of myeloid lineage.

By “isolated cell” is meant a cell that is separated from the molecular and/or cellular components that naturally accompany the cell.

By “activates an immunoresponsive cell” is meant induction of signal transduction or changes in protein expression in the cell resulting in the initiation of an immune response. For example, when CD3 Chains cluster in response to ligand binding and immunoreceptor tyrosine-based inhibition motifs (ITAMs) a signal transduction cascade is produced. In certain embodiments, when an endogenous TCR or an exogenous CAR binds to an antigen, a formation of an immunological synapse occurs that includes clustering of many molecules near the bound receptor (e.g. CD4 or CD8, CD3γ/δ/ε/ζ, etc.). This clustering of membrane-bound signaling molecules allows for ITAM motifs contained within the CD3 chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T cell, NK cell) activation or concomitantly mediated through receptors including, but not limited to, CD28, CD137 (4-1BB), OX40, CD40, and ICOS. Receiving multiple stimulatory signals can be important to mount a robust and long-term T cell mediated immune response. T cells can quickly become inhibited and unresponsive to antigen. While the effects of these co-stimulatory signals may vary, they generally result in increased gene expression in order to generate long lived, proliferative, and anti-apoptotic T cells that robustly respond to antigen for complete and sustained eradication.

As used herein, the term “affinity” is meant as a measure of binding strength. Affinity can depend on the closeness of stereochemical fit between antibody combining sites and antigen determinants, on the size of the area of contact between them, and/or on the distribution of charged and hydrophobic groups. As used herein, the term “affinity” also includes “avidity”, which refers to the strength of the antigen-antibody bond after the formation of reversible complexes. Methods for calculating the affinity of an antibody for an antigen are known in the art, including, but not limited to, various antigen-binding experiments, e.g., functional assays (e.g., flow cytometry assay).

As used herein, the term “antigen heterogeneity” refers to the differential expression of a number of antigens (e.g., tumor antigens, e.g., CD33, CD70, CD312) which results in variation in the tumor cell phenotype and distribution of tumor antigen-positive cells.

As used herein, the term “low antigen density” refers to a target molecule (e.g., an antigen) having a cell surface density of less than about 5,000 molecules per cell. In certain embodiments, the low antigen density is a cell surface density that is less than about 4,000 molecules per cell, less than about 3,000 molecules per cell, less than about 2,000 molecules per cell, less than about 1,500 molecules per cell, less than about 1,000 molecules per cell, less than about 500 molecules per cell, less than about 200 molecules per cell, or less than about 100 molecules per cell. In certain embodiments, the low antigen density is a cell surface density is less than about 2,000 molecules per cell. In certain embodiments, the low antigen density is a cell surface density is less than about 1,500 molecules per cell. In certain embodiments, the low antigen density is a cell surface density is less than about 1,000 molecules per cell. In certain embodiments, the low antigen density is a cell surface density is between about 4,000 molecules per cell and about 2,000 molecules per cell, between about 2,000 molecules per cell and about 1,000 molecules per cell, between about 1,500 molecules per cell and about 1,000 molecules per cell, between about 2,000 molecules per cell and about 500 molecules per cell, between about 1,000 molecules per cell and about 200 molecules per cell, or between about 1,000 molecules per cell and about 100 molecules per cell.

As used herein, the term “low tumor cell frequency” refers to a target cell having a target cell frequency of less than about 50% per tumor. In certain embodiments, the low tumor cell frequency is less than about 40% per tumor, less than about 30% per tumor, less than about 20% per tumor, less than about 15% per tumor, less than about 10% per tumor, less than about 5% per tumor, less than about 2% per tumor, or less than about 1% per tumor. In certain embodiments, the low tumor cell frequency is less than about 2% per tumor. In certain embodiments, the low tumor cell frequency is less than about 1.5% per tumor. In certain embodiments, the low tumor cell frequency is less than about 1% per tumor. In certain embodiments, the low tumor cell frequency is between about 40% per tumor and about 20% per tumor, between about 20% per tumor and about 10% per tumor, between about 15% per tumor and about 10% per tumor, between about 20% per tumor and about 5% per tumor, between about 10% per tumor and about 2% per tumor, or between about 10% per tumor and about 1% per tumor.

As used herein, the term “substantially identical” or “substantially homologous” refers to a polypeptide or a nucleic acid molecule exhibiting at least about 50% identical or homologous to a reference amino acid sequence (for example, any of the amino acid sequences described herein) or a reference nucleic acid sequence (for example, any of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% identical or homologous to the amino acid sequence or the nucleic acid sequence used for comparison.

Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences (e.g., heavy and light chain variable region sequences of scFv703) disclosed herein. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., XBLAST and NBLAST) can be used.

As used herein, the term “a conservative sequence modification” refers to an amino acid modification that does not significantly affect or alter the binding characteristics of the presently disclosed polypeptides. Conservative modifications can include amino acid substitutions, additions and deletions. Modifications can be introduced into the presently disclosed polypeptides by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

By “analog” is meant a structurally related polypeptide or nucleic acid molecule having the function of a reference polypeptide or nucleic acid molecule.

The term “ligand” as used herein refers to a molecule that binds to a receptor. In certain embodiments, the ligand binds to a receptor on another cell, allowing for cell-to-cell recognition and/or interaction.

The term “constitutive expression” or “constitutively expressed” as used herein refers to expression or expressed under all physiological conditions.

By “disease” is meant any condition, disease, or disorder that damages or interferes with the normal function of a cell, tissue, or organ, e.g., neoplasm, and pathogen infection of a cell.

By “effective amount” is meant an amount sufficient to have a therapeutic effect. In certain embodiments, an “effective amount” is an amount sufficient to arrest, ameliorate, or inhibit the continued proliferation, growth, or metastasis (e.g., invasion, or migration) of a neoplasm.

By “endogenous” is meant a nucleic acid molecule or polypeptide that is normally expressed in a cell or tissue.

By “exogenous” is meant a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By “modulate” is meant positively or negatively alter. Exemplary modulations include a about 1%, about 2%, about 5%, about 10%, about 25%, about 50%, about 75%, or about 100% change.

By “increase” is meant to alter positively by at least about 5%. An alteration may be by about 5%, about 10%, about 25%, about 30%, about 50%, about 75%, about 100%, or more.

By “reduce” is meant to alter negatively by at least about 5%. An alteration may be by about 5%, about 10%, about 25%, about 30%, about 50%, about 75%, or even by about 100%.

The terms “isolated,” “purified,” or “biologically pure” refer to material that is free to varying degrees from components that normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “neoplasm” is meant a disease characterized by the pathological proliferation of a cell or tissue and its subsequent migration to or invasion of other tissues or organs. Neoplasm growth is typically uncontrolled and progressive, and occurs under conditions that would not elicit, or would cause cessation of, multiplication of normal cells. Neoplasm can affect a variety of cell types, tissues, or organs, including but not limited to an organ selected from bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasms include cancers, such as sarcomas, carcinomas, or plasmacytomas (malignant tumor of the plasma cells). In certain embodiments, the neoplasm is cancer.

By “reference” or “control” is meant a standard of comparison. For example, the level of scFv-antigen binding by a cell expressing a CAR and an scFv may be compared to the level of scFv-antigen binding in a corresponding cell expressing CAR alone.

By “secreted” is meant a polypeptide that is released from a cell via the secretory pathway through the endoplasmic reticulum, Golgi apparatus, and as a vesicle that transiently fuses at the cell plasma membrane, releasing the proteins outside of the cell.

By “signal sequence” or “leader sequence” is meant a peptide sequence (e.g., 5, 10, 15, 20, 25 or 30 amino acids) present at the N-terminus of newly synthesized proteins that directs their entry to the secretory pathway. Exemplary leader sequences include, but is not limited to, a human IL-2 signal sequence (e.g., a human IL-2 signal sequence comprising or consisting of the amino acid sequence set forth in SEQ ID NO: 8), a mouse IL-2 signal sequence (e.g., a mouse IL-2 signal sequence comprising or consisting of the amino acid sequence set forth in SEQ ID NO: 9); a human kappa leader sequence (e.g., a human kappa leader sequence comprising or consisting of the amino acid sequence set forth in SEQ ID NO: 10), a mouse kappa leader sequence (e.g., a mouse kappa leader sequence comprising or consisting of the amino acid sequence set forth in SEQ ID NO: 11); a human CD8 leader sequence (e.g., a human CD8 leader sequence comprising or consisting of the amino acid sequence set forth in SEQ ID NO: 12); a truncated human CD8 signal peptide (e.g., a truncated human CD8 signal peptide comprising or consisting of the amino acid sequence set forth in SEQ ID NO: 13); a human albumin signal sequence (e.g., a human albumin signal sequence comprising or consisting of the amino acid sequence set forth in SEQ ID NO: 14); and a human prolactin signal sequence (e.g., a human prolactin signal sequence comprising or consisting of the amino acid sequence set forth in SEQ ID NO: 15). SEQ ID Nos: 8-15 are provided below.

[SEQ ID NO: 8]
MYRMQLLSCIALSLALVTNS
[SEQ ID NO: 9]
MYSMQLASCVTLTLVLLVNS
[SEQ ID NO: 10]
METPAQLLFLLLLWLPDTTG
[SEQ ID NO: 11]
METDTLLLWVLLLWVPGSTG
[SEQ ID NO: 12]
MALPVTALLLPLALLLHAARP
[SEQ ID NO: 13]
MALPVTALLLPLALLLHA
[SEQ ID NO: 14]
MKWVTFISLLFSSAYS
[SEQ ID NO: 15]
MDSKGSSQKGSRLLLLLVVSNLLLCQGVVS

By “soluble” is meant a polypeptide that is freely diffusible in an aqueous environment (e.g., not membrane bound).

By “specifically binds” is meant a polypeptide or fragment thereof that recognizes and binds to a biological molecule of interest (e.g., a polypeptide), but which does not substantially recognize and bind other molecules in a sample, for example, a biological sample, which naturally includes a presently disclosed polypeptide.

The term “tumor antigen” as used herein refers to an antigen (e.g., a polypeptide) that is uniquely or differentially expressed on a tumor cell compared to a normal or non-neoplastic cell. In certain embodiments, a tumor antigen includes any polypeptide expressed by a tumor that is capable of activating or inducing an immune response via an antigen recognizing receptor or capable of suppressing an immune response via receptor-ligand binding.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. The term “immunocompromised” as used herein refers to a subject who has an immunodeficiency. The subject is very vulnerable to opportunistic infections, infections caused by organisms that usually do not cause disease in a person with a healthy immune system, but can affect people with a poorly functioning or suppressed immune system.

The terms “comprises”, “comprising”, and are intended to have the broad meaning ascribed to them in U.S. Patent Law and can mean “includes”, “including” and the like.

Other aspects of the presently disclosed subject matter are described in the following disclosure and are within the ambit of the presently disclosed subject matter.

### 2. Cells

The presently disclosed subject matter provides cells comprising a chimeric receptor disclosed herein. In certain embodiments, the cell is selected from the group consisting of cells of lymphoid lineage and cells of myeloid lineage. In certain embodiments, the cell is an immunoresponsive cell. In certain embodiments, the immunoresponsive cell is a cell of lymphoid lineage.

In certain embodiments, the cell is a cell of the lymphoid lineage. Cells of the lymphoid lineage can provide production of antibodies, regulation of cellular immune system, detection of foreign agents in the blood, detection of cells foreign to the host, and the like. Non-limiting examples of cells of the lymphoid lineage include T cells, Natural Killer (NK) cells, B cells, dendritic cells, stem cells from which lymphoid cells may be differentiated. In certain embodiments, the stem cell is a pluripotent stem cell (e.g., embryonic stem cell).

In certain embodiments, the cell is an NK cell. Natural Killer (NK) cells can be lymphocytes that are part of cell-mediated immunity and act during the innate immune response. NK cells do not require prior activation in order to perform their cytotoxic effect on target cells. In certain embodiments, the NK cell is derived from an induced pluripotent stem cell (iPSC). Types of human lymphocytes of the presently disclosed subject matter include, without limitation, peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli, M. C., et al. 2000 J Immunol 164:495-504; Panelli, M. C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G. A., et al. 2003 Blood 102:2498-2505 (disclosing selectively in vitro-expanded antigen-specific peripheral blood leukocytes employing artificial antigen-presenting cells (AAPCs) or pulsed dendritic cells).

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), two types of effector memory T cells: e.g., T_{EM} cells and T_{EMRA} cells, Regulatory T cells (also known as suppressor T cells), tumor-infiltrating lymphocyte (TIL), virus-specific T cells (VST), Natural Killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of a presently disclosed chimeric receptor. Additionally, a patient's own T cells may be genetically modified to target specific antigens through the introduction of an antigen-recognizing receptor, e.g., a CAR or a TCR. The T cell can be a CD4⁺ T cell or a CD8⁺ T cell. In certain embodiments, the T cell is a CD4⁺ T cell. In certain embodiments, the T cell is a CD8⁺ T cell. In certain embodiments, the CD8⁺ T cell is CD4 independent. In certain embodiments, the T cell is derived from an induced pluripotent stem cell (iPSC). In certain embodiments, the T cell is a CD8⁺ T cell that is CD4 independent, and the CD8⁺ T cell is derived from an iPSC.

In certain embodiments, the CD8⁺ T cell is an NKG2C⁺CD8⁺ T cell. NKG2C⁺CD8⁺ T cells can downregulate genes associated with T cell identity (e.g., BCL11B) and express several NK cell markers (e.g., CD56, KIR). NKG2C⁺CD8⁺ T cells have a restricted TCR repertoire, are resistant to PD-1 upregulation, and have an intrinsic capacity to recognize diseased cells. Sottile et al., describes the characterization of the NKG2C+CD8+ T cell type (Sottile et al., Science Immunology (2021); 6 (63): 1-17, the content of which is incorporated by reference in its entirety). In certain embodiments, the NKG2C⁺CD8⁺ T cell is derived from an induced pluripotent stem cell (iPSC).

In certain embodiments, the T cell is a tumor-infiltrating lymphocyte (TIL). TILs are lymphocytes that have left the bloodstream and migrated towards a tumor. TILs can be found in the tumor stroma and within the tumor itself.

In certain embodiments, the cell (e.g., an NK cell or an NKG2C⁺CD8⁺ T cell) is autologous. In certain embodiments, the cell (e.g., an NK cell or an NKG2C⁺CD8⁺ T cell) is non-autologous. In certain embodiments, the cell (e.g., an NK cell or an NKG2C⁺CD8⁺ T cell) is allogeneic. In certain embodiments, the cell (e.g., an NK cell or an NKG2C+CD8⁺ T cell) is derived in vitro from an engineered progenitor or stem cell.

In certain embodiments, the cell is a cell of the myeloid lineage. Non-limiting examples of cells of the myeloid lineage include monocytes, macrophages, neutrophils, basophils, eosinophils, erythrocytes, megakaryocytes, and stem cells from which myeloid cells may be differentiated.

In certain embodiments, the stem cell is a pluripotent stem cell (e.g., an embryonic stem cell or an induced pluripotent stem cell).

### 2.1. Chimeric Receptors

In certain embodiments the chimeric receptor comprises an extracellular antigen-binding domain (e.g., an scFv) that binds to a target antigen. In certain embodiments, the chimeric receptor further comprises a hinge/spacer region. In certain embodiments, the chimeric receptor further comprises a transmembrane domain.

In certain embodiments, the chimeric receptor does not deliver an activation signal to a cell. In certain embodiments, the chimeric receptor does not deliver a co-stimulatory signal to a cell. In certain embodiments, the chimeric receptor does not deliver an activation signal to a cell and a co-stimulatory signal to a cell. In certain embodiments, the chimeric receptor is designated as “CAR-DEL”.

In certain embodiments, the chimeric receptor does not deliver an activation signal to a cell. In certain embodiments, the chimeric receptor does not deliver a co-stimulatory signal to a cell. In certain embodiments, the chimeric receptor does not deliver to a cell both: (1) an activation signal to a cell; and (2) a co-stimulatory signal. Without being bound by theory, the presently disclosed subject matter is directed to chimeric receptors, e.g., a CAR-DEL chimeric receptor, that functions as an adhesion molecule which, when expressed on the cell membrane of an immunoresponsive cell (e.g., an effector cell), brings the cell expressing the chimeric receptor, e.g., a CAR-DEL chimeric receptor, in close proximity to the tumor. The effector cell (e.g., an NK cell or NKG2C⁺CD8⁺ T cell) expressing the chimeric receptor then exerts its own innate mechanism to kill the tumor cell. Therefore, an effector cell (e.g., an NK cell or NKG2C⁺CD8⁺ T cell) can be activated by being placed in close proximity to the target cell while the chimeric receptor, e.g., a CAR-DEL chimeric receptor, does not, itself, deliver an activation signal.

In certain embodiments, the chimeric receptor (e.g., a CAR-DEL chimeric receptor) does not deliver an activation signal upon binding to an antigen. In certain embodiments, the chimeric receptor (e.g., a CAR-DEL chimeric receptor) does not deliver a co-stimulatory signal upon binding to an antigen. In certain embodiments, the chimeric receptor (e.g., a CAR-DEL chimeric receptor) does not deliver an activation signal and a co-stimulatory signal upon binding to an antigen.

In certain embodiments, the chimeric receptor (e.g., a CAR-DEL chimeric receptor) comprises an intracellular polypeptide that does not deliver a co-stimulatory signal. In certain embodiments, the chimeric receptor comprises an intracellular polypeptide that does not deliver an activation signal. In certain embodiments, the chimeric receptor comprises an intracellular polypeptide that does not deliver both: (1) a co-stimulatory signal; and (2) an activation signal. In certain embodiments, the chimeric receptor comprises an intracellular polypeptide that lacks a CD3ζ polypeptide.

In certain embodiments, the chimeric receptor is a CAR-DEL chimeric receptor that lacks a CD3ζ polypeptide.

In certain embodiments, the chimeric receptor, e.g., a CAR-DEL chimeric receptor, targets an antigen. The antigen can be a tumor antigen or a pathogen antigen.

### 2.1.1. Antigen

In certain embodiments, the antigen is a tumor antigen. Any tumor antigen (antigenic peptide) can be used in the tumor-related embodiments described herein. Sources of antigen include, but are not limited to, cancer proteins. The antigen can be expressed as a peptide or as an intact protein or a portion thereof. The intact protein or portion thereof can be native or mutagenized. Non-limiting examples of tumor antigens include CD33, CD19, CD70, IL1RAP, ABCG2, AChR, ACKR6, ADAMTS13, ADGRE2, ADGRE2 (EMR2), ADORA3, ADRAID, AGER, ALS2, an antigen of a cytomegalovirus (CMV) infected cell (e.g. a cell surface antigen), ANO9, AQP2, ASIC3, ASPRV1, ATP6V0A4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD276, CD30, CD300LF, CD312, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGFIR, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGRIA, FKBP1B, FLRT1, folate receptor-a, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAMI, IGFBP3, IL10RB, IL20RB, IL23R, ILDR1, Interleukin-13 receptor subunit alpha-2 (IL-13Ra2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, K-light chain, LICAM, LAXI, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNNID, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TASIR3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

In certain embodiments, the antigen is selected from the group consisting of CD312, CLEC12A, CD33, CD123, IL1RAP, SIGLEC-6, GRP78, TIM3, CD70, CD20, CD22, CD19, GPRC5D, SLAMF7, BCMA, CD276, and CAIX. In certain embodiments, the antigen is CD33.

In certain embodiments, the antigen is a pathogen antigen. In certain embodiments, the pathogen antigen is an antigen of a virus or a bacteria. Non-limiting examples of viruses include, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Naira viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2-parenterally transmitted (i.e. Hepatitis C); Norwalk and related viruses, and astroviruses).

Non-limiting examples of bacteria include Pasteurella, Staphylococci, Streptococcus, Escherichia coli, Pseudomonas species, and Salmonella species. Specific examples of infectious bacteria include but are not limited to, Helicobacter pyloris, Borelia burgdorferi, Legionella, Legionella pneumophilia, Mycobacteria sps (e.g. M. tuberculosis, M. avium, M. intracellulare, M. kansaii, M. gordonae, M. leprae), Staphylococcus aureus, Staphylococcus epidermidis, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Campylobacter jejuni, Enterococcus sp., Haemophilus influenzae, Bacillus antracis, Corynebacterium diphtheriae, corynebacterium sp., Erysipelothrix rhusiopathiae, Clostridium spp., Clostridium perfringers, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, and Actinomyces israelli. Mycoplasma, Pseudomonas aeruginosa, Pseudomonas fluorescens, Corynobacteria diphtheriae, Bartonella henselae, Bartonella quintana, Coxiella burnetii, chlamydia, shigella, Yersinia enterocolitica, Yersinia pseudotuberculosis, Listeria monocytogenes, Mycoplasma spp., Vibrio cholerae, Borrelia, Francisella, Brucella melitensis, Proteus mirabilis, and Proteus.

In certain embodiments, the pathogen antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

### 2.1.1.1 CD33

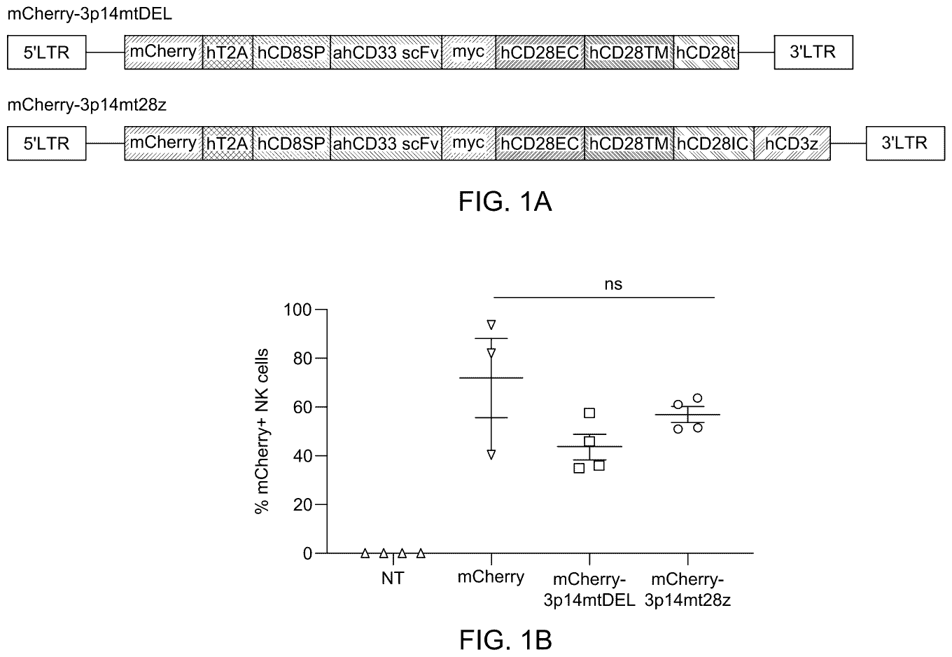

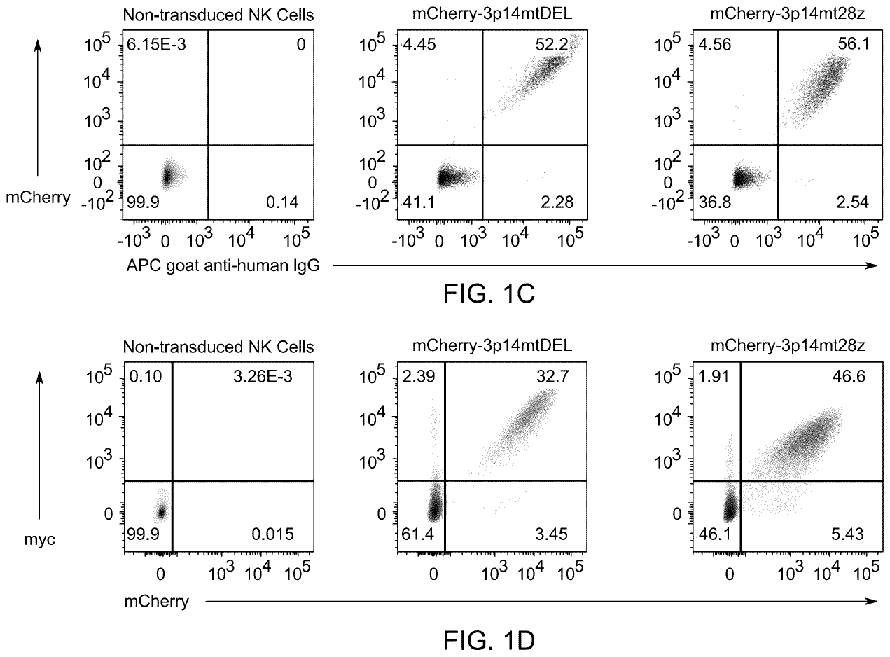

In certain embodiments, the antigen is CD33. CD33 is a single pass transmembrane molecule and a member of the sialic acid-binding immunoglobulin (Ig)-like lectin (Siglec) family. CD33 consists of two extracellular domains with immunoglobulin-like folds, IgV and IgC2 (see FIG. 1A). CD33 has 3 isoforms produced via alternate splicing, with isoform 3 missing the IgV domain (Ehninger et al., Blood Cancer J4, e218 (2014); Sanford et al., Leuk Lymphoma 57, 1965-1968 (2016); and Haubner et al., Leukemia 33, 64-74 (2019)) Recent studies showed that about 50% of AML patients have a CD33 single-nucleotide polymorphism (SNP) (rs12459419 C>T) that leads to the expression of an alternatively spliced CD33 isoform lacking exon 2, resulting in the elimination of the IgV domain (Bakker et al., Cancer Res 64, 8443-8450 (2004)). In these patients, gemtuzumab ozogamicin (GO), an antibody-drug conjugate (ADC) targeting CD33, had no impact and increased relapse risk likely due to the inability of this ADC to kill AML cells expressing this IgV-lacking CD33 isoform. This issue is bound to be encountered by all the currently clinically available CD33-targeting products given their epitopes in the IgV domain (see FIG. 1B) (Perna et al., Cancer Cell 32, 506-519 e505 (2017)).

In certain embodiments, the antigen recognizing receptor binds to human CD33. In certain embodiments, the human CD33 comprises or consists of the amino acid sequence with a UniProt Reference No: P20138-1 (SEQ ID NO: 27) or a fragment thereof. SEQ ID NO: 27 is provided below. In certain embodiments, the CD33 comprises an extracellular domain, a transmembrane domain, and a cytoplasmic domain. In certain embodiments, the extracellular domain comprises or consists of amino acids 18 to 259 of SEQ ID NO: 27. In certain embodiments, the transmembrane domain comprises or consists of amino acids 260 to 282 of SEQ ID NO: 27. In certain embodiments, the cytoplasmic domain comprises or consists of amino acids 283 to 364 of SEQ ID NO: 27.

[SEQ ID NO: 27]
MPLLLLLPLLWAGALAMDPNFWLQVQESVTVQEGLCVLVPCTFFHPIPYYDKNSPVHGYWFREG
AIISRDSPVATNKLDQEVQEETQGRFRLLGDPSRNNCSLSIVDARRRDNGSYFFRMERGSTKYS
YKSPQLSVHVTDLTHRPKILIPGTLEPGHSKNLTCSVSWACEQGTPPIFSWLSAAPTSLGPRTT
HSSVLIITPRPQDHGTNLTCQVKFAGAGVTTERTIQLNVTYVPQNPTTGIFPGDGSGKQETRAG
VVHGAIGGAGVTALLALCLCLIFFIVKTHRRKAARTAVGRNDTHPTTGSASPKHQKKSKLHGPT
ETSSCSGAAPTVEMDEELHYASLNFHGMNPSKDTSTEYSEVRTQ

In certain embodiments, the CD33 comprises or consists of an amino acid sequence that is at least about 80%, at least about 85%, at least about 90%, at least about 95%, at least about 96%, at least about 97%, at least about 98%, or at least about 99%, at least about 100% identical to the amino acid sequence set forth in SEQ ID NO: 27 or a fragment thereof.

In certain embodiments, the antigen recognizing receptor binds to a portion of human CD33. In certain embodiments, the antigen recognizing receptor binds to the extracellular domain of CD33. In certain embodiments, the extracellular domain of CD33 comprises an Ig-like V-type domain and an Ig-like C2-type. In certain embodiments, the extracellular domain of CD33 comprises an Ig-like C2-type. In certain embodiments, the Ig-like V-type domain comprises or consists of amino acids 19 to 135 of SEQ ID NO: 27. In certain embodiments, the Ig-like C2-type domain comprises or consists of amino acids 145 to 228 of SEQ ID NO: 27.

### 2.1.2. Extracellular Antigen-Binding Domain

In certain embodiments, the extracellular antigen-binding domain of the chimeric receptor (for example, an scFv) binds to the antigen with a dissociation constant (K_{D}) of about 5×10⁻⁷ M or less, about 1×10⁻⁷ M or less, about 5×10⁻⁸ M or less, about 1×10⁻⁸ M or less, about 5×10⁻⁹ M or less, or about 1×10⁻⁹ M or less, or about 1×10⁻¹⁰ M or less. In certain embodiments, the extracellular antigen-binding domain of the chimeric receptor (for example, an scFv) binds to the antigen with a K_{D} of about 1×10⁻⁸ M or less.

Binding of the extracellular antigen-binding domain (for example, in an scFv) can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody, or an scFv) specific to the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March 1986, which is incorporated by reference herein). The radioactive isotope can be detected by such means as the use of a γ counter or a scintillation counter or autoradiography. In certain embodiments, the extracellular antigen-binding domain of the chimeric receptor is labeled with a fluorescent marker. Non-limiting examples of fluorescent markers include green fluorescent protein (GFP), red fluorescent protein (e.g., DsRed, mCherry, mKate), blue fluorescent protein (e.g., EBFP, EBFP2, Azurite, and mKalama1), cyan fluorescent protein (e.g., ECFP, Cerulean, and CyPet), and yellow fluorescent protein (e.g., YFP, Citrine, Venus, and Ypet).

The extracellular antigen-binding domain can comprise or be an scFv, a Fab (which is optionally crosslinked), or an F(ab)₂. In certain embodiments, any of the foregoing molecules may be comprised in a chimeric protein with a heterologous sequence to form the extracellular antigen-binding domain. In certain embodiments, the extracellular antigen-binding domain comprises or is an scFv. In certain embodiments, the scFv is a human scFv. In certain embodiments, the scFv is a humanized scFv. In certain embodiments, the scFv is a murine scFv.

In addition, the extracellular antigen-binding domain of the chimeric receptor (e.g., CAR-DEL) can comprise a leader or a signal peptide that directs the nascent protein into the endoplasmic reticulum. Signal peptide or leader can be essential if the chimeric receptor is to be glycosylated and anchored in the cell membrane. The signal sequence or leader can be a peptide sequence (about 5, about 10, about 15, about 20, about 25, or about 30 amino acids long) present at the N-terminus of newly synthesized proteins that directs their entry to the secretory pathway. In certain embodiments, the signal peptide is covalently joined to the 5′ terminus (N-terminus) of the extracellular antigen-binding domain of the chimeric receptor. In certain embodiments, the signal peptide comprises a CD8 polypeptide, e.g., the chimeric receptor (e.g., CAR-DEL) comprises a truncated CD8 signal peptide. In certain embodiments, the signal peptide comprises or consists of the amino acid sequence set forth in SEQ ID NO: 13.

### 2.1.2.1. Exemplary Extracellular Antigen-Binding Domains

In certain embodiments, the chimeric receptor (e.g., CAR-DEL) comprises an extracellular antigen-binding domain that binds to CD33.

In certain embodiments, the extracellular antigen-binding domain of the chimeric receptor (e.g., CAR-DEL) comprises a heavy chain variable region (V_{H}) comprising a CDR1 comprising the amino acid sequence set forth in SEQ ID NO: 28 or a conservative modification thereof, a CDR2 comprising the amino acid sequence set forth in SEQ ID NO: 29 or a conservative modification thereof, and a CDR3 comprising the amino acid sequence set forth in SEQ ID NO: 30 or a conservative modification thereof. SEQ ID Nos: 28-30 are provided in Table 1.

In certain embodiments, the extracellular antigen-binding domain of the chimeric receptor (e.g., CAR-DEL) comprises a light chain variable region (V_{L}) comprising a CDR1 comprising the amino acid sequence set forth in SEQ ID NO: 31 or a conservative modification thereof, a CDR2 comprising the amino acid sequence set forth in SEQ ID NO: 32 or a conservative modification thereof, and a CDR3 comprising the amino acid sequence set forth in SEQ ID NO: 33 or a conservative modification thereof. SEQ ID Nos: 31-33 are provided in Table 1.

In certain embodiments, the extracellular antigen-binding domain (e.g., an scFv) comprises a V_{H} comprising a CDR1 comprising the amino acid sequence set forth in SEQ ID NO: 28 or a conservative modification thereof, a CDR2 comprising the amino acid sequence set forth in SEQ ID NO: 29 or a conservative modification thereof, and a CDR3 comprising the amino acid sequence set forth in SEQ ID NO: 30 or a conservative modification thereof; and a V_{L} comprising a CDR1 comprising the amino acid sequence set forth in SEQ ID NO: 31 or a conservative modification thereof, a CDR2 comprising the amino acid sequence set forth in SEQ ID NO: 32 or a conservative modification, and a CDR3 comprising the amino acid sequence set forth in SEQ ID NO: 33 or a conservative modification thereof.

In certain embodiments, the extracellular antigen-binding domain (e.g., an scFv) comprises a V_{H} comprising a CDR1 comprising the amino acid sequence set forth in SEQ ID NO: 28, a CDR2 comprising the amino acid sequence set forth in SEQ ID NO: 29, and a CDR3 comprising the amino acid sequence set forth in SEQ ID NO: 30; and a V_{L} comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 34. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 34. In certain embodiments, the extracellular antigen-binding domain comprises a V_{H} comprising the amino sequence set forth in SEQ ID NO: 34. An exemplary nucleotide sequence encoding the amino acid sequence of SEQ ID NO: 34 is set forth in SEQ ID NO: 35. SEQ ID Nos: 34 and 35 are provided in Table 1 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 36. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 36. In certain embodiments, the extracellular antigen-binding domain comprises a V_{L} comprising the amino sequence set forth in SEQ ID NO: 36. An exemplary nucleotide sequence encoding the amino acid sequence of SEQ ID NO: 36 is set forth in SEQ ID NO: 37. SEQ ID Nos: 36 and 37 are provided in Table 1 below.

In certain embodiments, the extracellular antigen-binding domain (e.g., an scFv) comprises a V_{H} comprising the amino acid sequence set forth in SEQ ID NO: 34, and a V_{L} comprising the amino acid sequence set forth in SEQ ID NO: 36. In certain embodiments, the extracellular antigen-binding domain is an scFv. In certain embodiments, the scFv is designated as “3-P14”. In certain embodiments, the V_{H} and V_{L} are linked via a linker. In certain embodiments, the linker comprises the amino acid sequence set forth in SEQ ID NO: 1, SEQ ID NO: 2, SEQ ID NO: 3, SEQ ID NO: 4, SEQ ID NO: 5, SEQ ID NO: 6, or SEQ ID NO: 7.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, the extracellular antigen-binding domain is an scFv, the variable regions are positioned from the N- to the C-terminus: V_{L}-V_{H}.

TABLE 1

| CDRs | 1 | 2 | 3 |
|---|---|---|---|
| V_{H} | GFTFSTYA | ISGRGGST | AGRGDYYYYYGMDV |
| | [SEQ ID NO: 28] | [SEQ ID NO: 29] | [SEQ ID NO: 30] |
| V_{L} | QSLVYSDGNTY | KIS | MQSQFPHT |
| | [SEQ ID NO: 31] | [SEQ ID NO: 32] | [SEQ ID NO: 33] |

| | |
|---|---|
| Full V_{H} | EVQLLESGGGLVQPGGSLRLSCAASGFTFSTYAMSWVRQAPGKGLEWVSAISGRG |
| | GSTYYTDSVKGRFTISRDNSKNTVSLQMNSLRAEDTAVYYCAGRGDYYYYYGMDV |
| | WGQGTTVTVSA [SEQ ID NO: 34] |
| Full V_{L} | DIVMTQSPLSSPVTLGQPASFSCRSSQSLVYSDGNTYLSWLQQRPGQPPRLLIYK |
| | ISNRFSGVPDRFSGSGAGTDFTLKISRVEAEDVGVYYCMQSTQFPHTFGQGTKLE |
| | IK [SEQ ID NO: 36] |
| DNA | GAGGTGCAGCTGTTGGAGTCTGGGGGAGGCTTGGTCCAGCCTGGGGGGTCCCTGA |
| for | GACTCTCCTGTGCAGCCTCTGGATTCACCTTTAGCACCTATGCCATGAGCTGGGT |
| Full V_{H} | CCGCCAGGCTCCAGGGAAGGGGCTGGAGTGGGTCTCAGCTATTAGTGGTCGTGGT |
| | GGTAGCACATACTACACAGACTCCGTGAAGGGCCGGTTCACCATCTCCAGAGACA |
| | ATTCCAAGAACACGGTGTCTCTGCAAATGAACAGCCTGAGAGCCGAGGACACGGC |
| | CGTATATTACTGTGCGGGCCGGGGAGATTACTACTACTACTACGGTATGGACGTC |
| | TGGGGCCAAGGGACCACGGTCACCGTCTCCGCA [SEQ ID NO: 35] |
| DNA | GATATTGTGATGACCCAGAGTCCACTCTCCTCACCTGTCACCCTTGGACAGCCGG |
| for | CCTCCTTCTCCTGCAGGTCTAGTCAAAGCCTCGTATACAGTGATGGAAACACCTA |
| Full V_{L} | CTTGAGTTGGCTTCAGCAGAGGCCAGGCCAGCCTCCAAGACTCCTAATTTATAAG |
| | ATTTCTAACCGGTTCTCTGGGGTCCCAGACAGATTCAGTGGCAGTGGGGCAGGGA |
| | CAGATTTCACACTGAAAATCAGCAGGGTGGAAGCTGAGGATGTCGGGGTTTATTA |
| | CTGCATGCAATCTACACAATTTCCTCACACTTTTGGCCAGGGGACCAAGCTGGAG |
| | ATCAAA [SEQ ID NO: 37] |

The V_{H} and/or V_{L} amino acid sequences having at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to a specific sequence (e.g., SEQ ID Nos: 34 or 36) may contain substitutions (e.g., conservative substitutions), insertions, or deletions relative to the specified sequence(s), but retain the ability to bind to a target antigen (e.g., CD33). In certain embodiments, a total of 1 to 10 amino acids are substituted, inserted, and/or deleted in a specific sequence (e.g., SEQ ID Nos: 34 or 36). In certain embodiments, substitutions, insertions, or deletions occur in regions outside the CDRs (e.g., in the FRs) of the extracellular antigen-binding domain. In certain embodiments, the extracellular antigen-binding domain of the chimeric receptor comprises V_{H} and/or V_{L} sequence selected from SEQ ID Nos: 34 or 36, including post-translational modifications of that sequence (SEQ ID NO: 34 or 36).

Additional examples of extracellular antigen-binding domain that binds to CD33 can be found in International Patent Publication No. WO 2023/034560, which is incorporated by reference in its entirety.

### 2.1.3. Transmembrane Domain and Hinge/Spacer Region

In certain embodiments, the transmembrane domain of the chimeric receptor (e.g., CAR-DEL) comprises a hydrophobic alpha helix that spans at least a portion of the membrane. Different transmembrane domains result in different receptor stability. In accordance with the presently disclosed subject matter, the transmembrane domain of the chimeric receptor (e.g., CAR-DEL) can comprise a native or modified transmembrane domain of CD8 or a fragment thereof, a native or modified transmembrane domain of CD28 or a fragment thereof, a native or modified transmembrane domain of CD32 or a fragment thereof, a native or modified transmembrane domain of CD4 or a fragment thereof, a native or modified transmembrane domain of 4-1BB or a fragment thereof, a native or modified transmembrane domain of OX40 or a fragment thereof, a native or modified transmembrane domain of ICOS or a fragment thereof, a native or modified transmembrane domain of CD84 or a fragment thereof, a native or modified transmembrane domain of CD166 or a fragment thereof, a native or modified transmembrane domain of CD8a or a fragment thereof, a native or modified transmembrane domain of CD8b or a fragment thereof, a native or modified transmembrane domain of ICAM-1 or a fragment thereof, a native or modified transmembrane domain of CTLA-4 or a fragment thereof, a native or modified transmembrane domain of CD27 or a fragment thereof, a native or modified transmembrane domain of CD40 or a fragment thereof, NKGD2 or a fragment thereof, a native or modified transmembrane domain of PD-1 or a fragment thereof, a native or modified transmembrane domain of LAG-3 or a fragment thereof, a native or modified transmembrane domain of 2B4 or a fragment thereof, a native or modified transmembrane domain of BTLA or a fragment thereof, a native or modified transmembrane domain of DAP12 or a fragment thereof, a native or modified transmembrane domain of DAP10 or a fragment thereof, a native or modified transmembrane domain of CD16 or a fragment thereof, a native or modified transmembrane domain of DNAM1 or a fragment thereof, or a combination thereof.

In certain embodiments, the transmembrane domain of a presently disclosed chimeric receptor (e.g., CAR-DEL) comprises a CD28 polypeptide (e.g., a transmembrane domain of CD28 or a fragment thereof).

In certain embodiments, the transmembrane domain of the chimeric receptor (e.g., CAR-DEL) comprises a transmembrane domain of human CD28 or a fragment thereof. In certain embodiments, the CD28 polypeptide comprises or consists of an amino acid sequence that is at least about 85%, about 90%, about 95%, about 96%, about 97%, about 98%, about 99%, or 100% homologous or identical to the amino acid sequence having an NCBI Reference No: NP 006130 (SEQ ID No: 40) or a fragment thereof, and/or may optionally comprise up to one or up to two or up to three conservative amino acid substitutions. In certain non-limiting embodiments, the CD28 polypeptide comprises or consists of an amino acid sequence that is a consecutive portion of SEQ ID NO: 40 which is at least 20, or at least 30, or at least 40, or at least 50, and up to 220 amino acids in length. In certain embodiments, the CD28 polypeptide comprises or consists of an amino acid sequence of amino acids 1 to 220, 1 to 50, 50 to 100, 100 to 150, 150 to 200, 153 to 179, or 180 to 190 of SEQ ID NO: 40. In certain embodiments, the transmembrane domain of the chimeric receptor (e.g., CAR-DEL) comprises a CD28 polypeptide comprising or consisting of amino acids 153 to 179 of SEQ ID NO: 40. SEQ ID NO: 40 is provided below:

[SEQ ID NO: 40]
MLRLLLALNLFPSIQVTGNKILVKQSPMLVAYDNAVNLSCKYSYNLFSR
EFRASLHKGLDSAVEVCVVYGNYSQQLQVYSKTGFNCDGKLGNESVTFY
LQNLYVNQTDIYFCKIEVMYPPPYLDNEKSNGTIIHVKGKHLCPSPLFP
GPSKPFWVLVVVGGVLACYSLLVTVAFIIFWVRSKRSRLLHSDYMNMTP
RRPGPTRKHYQPYAPPRDFAAYRS

An exemplary nucleotide sequence encoding amino acids 153 to 179 of SEQ ID NO: 40 is set forth in SEQ ID NO: 41, which is provided below.

[SEQ ID NO: 41]
TTTTGGGTGCTGGTGGTGGTTGGTGGAGTCCTGGCTTGCTATAGCTTGC
TAGTAACAGTGGCCTTTATTATTTTCTGGGTG

In certain embodiments, the transmembrane domain of the chimeric receptor (e.g., CAR-DEL) comprises a CD28 polypeptide (e.g., a transmembrane domain of mouse CD28 or a fragment thereof). In certain embodiments, the CD28 polypeptide comprises or consists of an amino acid sequence that is at least about 85%, about 90%, about 95%, about 96%, about 97%, about 98%, about 99%, or 100% homologous or identical to the amino acid sequence having a NCBI Reference No: NP_031668.3 (SEQ ID No: 42) or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 42, which is at least 20, or at least 30, or at least 40, or at least 50, and up to 218 amino acids in length. In certain embodiments, the CD28 polypeptide comprises or consists of an amino acid sequence of amino acids 1 to 218, 1 to 50, 50 to 100, 100 to 150, 150 to 200, 151 to 177, or 178 to 188 of SEQ ID NO: 42. In certain embodiments, the transmembrane domain of the chimeric receptor (e.g., a CAR-DEL chimeric receptor) comprises a CD28 polypeptide comprising or consisting of amino acids 151 to 177 of SEQ ID NO: 42. SEQ ID NO: 42 is provided below:

[SEQ ID NO: 42]
MTLRLLFLALNFFSVQVTENKILVKQSPLLVVDSNEVSLSCRYSYNLLA
KEFRASLYKGVNSDVEVCVGNGNFTYQPQFRSNAEFNCDGDFDNETVTF
RLWNLHVNHTDIYFCKIEFMYPPPYLDNERSNGTIIHIKEKHLCHTQSS
PKLFWALVVVAGVLFCYGLLVTVALCVIWINSRRNRLLQSDYMNMTPRR
PGLTRKPYQPYAPARDFAAYRP

In certain embodiments, the transmembrane domain of the chimeric receptor (e.g., a CAR-DEL chimeric receptor) comprises a transmembrane domain of human CD16 or a fragment thereof. In certain embodiments, the CD16 polypeptide comprises or consists of an amino acid sequence that is at least about 85%, about 90%, about 95%, about 96%, about 97%, about 98%, about 99%, or 100% homologous or identical to the amino acid sequence having a UniProt Reference No: P08637 (SEQ ID No: 103) or a fragment thereof, and/or may optionally comprise up to one or up to two or up to three conservative amino acid substitutions. In certain non-limiting embodiments, the CD16 polypeptide comprises or consists of an amino acid sequence that is a consecutive portion of SEQ ID NO: 103 which is at least 10, or at least 20, or at least 30, or at least 40, or at least 50, and up to 254 amino acids in length. In certain embodiments, the CD16 polypeptide comprises or consists of an amino acid sequence of amino acids 1 to 254, 1 to 50, 50 to 100, 100 to 150, 150 to 200, 200 to 233, 209 to 233, or 209 to 229 of SEQ ID NO: 103. In certain embodiments, the transmembrane domain of the chimeric receptor (e.g., a CAR-DEL chimeric receptor) comprises a CD16 polypeptide comprising or consisting of amino acids 209 to 229 of SEQ ID NO: 103. SEQ ID NO: 103 is provided below:

[SEQ ID NO: 103]
MWQLLLPTALLLLVSAGMRTEDLPKAVVELEPQWYRVLEKDSVTLKCQGA
YSPEDNSTQWFHNESLISSQASSYFIDAATVDDSGEYRCQTNLSTLSDPV
QLEVHIGWLLLQAPRWVFKEEDPIHLRCHSWKNTALHKVTYLQNGKGRKY
FHHNSDFYIPKATLKDSGSYFCRGLFGSKNVSSETVNITITQGLAVSTIS
SFFPPGYQVSFCLVMVLLFAVDTGLYFSVKTNIRSSTRDWKDHKFKWRKD
PQDK

In certain embodiments, the transmembrane domain of the chimeric receptor (e.g., a CAR-DEL chimeric receptor) comprises a CD16 polypeptide (e.g., a transmembrane domain of mouse CD16 or a fragment thereof). In certain embodiments, the CD16 polypeptide comprises or consists of an amino acid sequence that is at least about 85%, about 90%, about 95%, about 96%, about 97%, about 98%, about 99%, or 100% homologous or identical to the amino acid sequence having a UniProt Reference No: P08508 (SEQ ID No: 104) or a fragment thereof, and/or may optionally comprise up to one or up to two or up to three conservative amino acid substitutions. In certain embodiments, the CD16 polypeptide comprises or consists of an amino acid sequence that is a consecutive portion of SEQ ID NO: 104, which is at least 10, or at least 20, or at least 30, or at least 40, or at least 50, and up to 261 amino acids in length. In certain embodiments, the CD16 polypeptide comprises or consists of an amino acid sequence of amino acids 1 to 241, 1 to 50, 50 to 100, 100 to 150, 150 to 200, 200 to 261, or 216 to 235 of SEQ ID NO: 104. In certain embodiments, the transmembrane domain of the chimeric receptor (e.g., a CAR-DEL chimeric receptor) comprises a CD16 polypeptide comprising or consisting of amino acids 216 to 235 of SEQ ID NO: 104. SEQ ID NO: 104 is provided below:

[SEQ ID NO: 104]
MFQNAHSGSQWLLPPLTILLLFAFADRQSAALPKAVVKLDPPWIQVLKED
MVTLMCEGTHNPGNSSTQWFHNGRSIRSQVQASYTFKATVNDSGEYRCQM
EQTRLSDPVDLGVISDWLLLQTPQRVFLEGETITLRCHSWRNKLLNRISF
FHNEKSVRYHHYKSNFSIPKANHSHSGDYYCKGSLGSTQHQSKPVTITVQ
DPATTSSISLVWYHTAFSLVMCLLFAVDTGLYFYVRRNLQTPREYWRKSL
SIRKHQAPQDK

In certain embodiments, the transmembrane domain of the chimeric receptor (e.g., a CAR-DEL chimeric receptor) comprises a transmembrane domain of human DAP10 or a fragment thereof. In certain embodiments, the DAP10 polypeptide comprises or consists of an amino acid sequence that is at least about 85%, about 90%, about 95%, about 96%, about 97%, about 98%, about 99%, or 100% homologous or identical to the amino acid sequence having a UniProt Reference No: Q9UBK5 (SEQ ID No: 105) or a fragment thereof, and/or may optionally comprise up to one or up to two or up to three conservative amino acid substitutions. In certain non-limiting embodiments, the DAP10 polypeptide comprises or consists of an amino acid sequence that is a consecutive portion of SEQ ID NO: 105 which is at least 10, or at least 20, or at least 30, or at least 40, or at least 50, and up to 93 amino acids in length. In certain embodiments, the DAP10 polypeptide comprises or consists of an amino acid sequence of amino acids 1 to 93, 10 to 69, 20 to 69, 30 to 69, 40 to 69, or 49 to 69 of SEQ ID NO: 105. In certain embodiments, the transmembrane domain of the chimeric receptor (e.g., a CAR-DEL chimeric receptor) comprises a DAP10 polypeptide comprising or consisting of amino acids 49 to 69 of SEQ ID NO: 105. SEQ ID NO: 105 is provided below:

[SEQ ID NO: 105]
MIHLGHILFLLLLPVAAAQTTPGERSSLPAFYPGTSGSCSGCGSLSLPLL
AGLVAADAVASLLIVGAVFLCARPRRSPAQEDGKVYINMPGRG

In certain embodiments, the transmembrane domain of the chimeric receptor (e.g., a CAR-DEL chimeric receptor) comprises a DAP10 polypeptide (e.g., a transmembrane domain of mouse DAP10 or a fragment thereof). In certain embodiments, the DAP10 polypeptide comprises or consists of an amino acid sequence that is at least about 85%, about 90%, about 95%, about 96%, about 97%, about 98%, about 99%, or 100% homologous or identical to the amino acid sequence having a UniProt Reference No: Q9QUJ0 (SEQ ID No: 106) or a fragment thereof, and/or may optionally comprise up to one or up to two or up to three conservative amino acid substitutions. In certain embodiments, the DAP10 polypeptide comprises or consists of an amino acid sequence that is a consecutive portion of SEQ ID NO: 106, which is at least 10, or at least 20, or at least 30, or at least 40, or at least 50, and up to 79 amino acids in length. In certain embodiments, the DAP10 polypeptide comprises or consists of an amino acid sequence of amino acids 1 to 56, 1 to 56, 10 to 56, 20 to 56, 30 to 56, 40 to 56, or 36 to 56 of SEQ ID NO: 106. In certain embodiments, the transmembrane domain of the chimeric receptor (e.g., a CAR-DEL chimeric receptor) comprises a DAP10 polypeptide comprising or consisting of amino acids 36 to 56 of SEQ ID NO: 106. SEQ ID NO: 106 is provided below:

[SEQ ID NO: 106]
MDPPGYLLFLLLLPVAASQTSAGSCSGCGTLSLPLLAGLVAADAVMSLLI
VGVVFVCMRPHGRPAQEDGRVYINMPGRG

In certain embodiments, the transmembrane domain of the chimeric receptor (e.g., a CAR-DEL chimeric receptor) comprises a transmembrane domain of human DAP12 or a fragment thereof. In certain embodiments, the DAP12 polypeptide comprises or consists of an amino acid sequence that is at least about 85%, about 90%, about 95%, about 96%, about 97%, about 98%, about 99%, or 100% homologous or identical to the amino acid sequence having a UniProt Reference No: 043914 (SEQ ID No: 107) or a fragment thereof, and/or may optionally comprise up to one or up to two or up to three conservative amino acid substitutions. In certain non-limiting embodiments, the DAP12 polypeptide comprises or consists of an amino acid sequence that is a consecutive portion of SEQ ID NO: 107 which is at least 10, or at least 20, or at least 30, or at least 40, or at least 50, and up to 113 amino acids in length. In certain embodiments, the DAP12 polypeptide comprises or consists of an amino acid sequence of amino acids 1 to 113, 10 to 61, 20 to 61, 30 to 61, 40 to 61, or 41 to 61 of SEQ ID NO: 107. In certain embodiments, the transmembrane domain of the chimeric receptor (e.g., a CAR-DEL chimeric receptor) comprises a DAP12 polypeptide comprising or consisting of amino acids 41 to 61 of SEQ ID NO: 107. SEQ ID NO: 107 is provided below:

[SEQ ID NO: 107]
MGGLEPCSRLLLLPLLLAVSGLRPVQAQAQSDCSCSTVSPGVLAGIVMGD
LVLTVLIALAVYFLGRLVPRGRGAAEAATRKQRITETESPYQELQGQRSD
VYSDLNTQRPYYK

In certain embodiments, the transmembrane domain of the chimeric receptor (e.g., a CAR-DEL chimeric receptor) comprises a DAP12 polypeptide (e.g., a transmembrane domain of mouse DAP12 or a fragment thereof). In certain embodiments, the DAP12 polypeptide comprises or consists of an amino acid sequence that is at least about 85%, about 90%, about 95%, about 96%, about 97%, about 98%, about 99%, or 100% homologous or identical to the amino acid sequence having a UniProt Reference No: 054885 (SEQ ID No: 108) or a fragment thereof, and/or may optionally comprise up to one or up to two or up to three conservative amino acid substitutions. In certain embodiments, the DAP12 polypeptide comprises or consists of an amino acid sequence that is a consecutive portion of SEQ ID NO: 108, which is at least 10, or at least 20, or at least 30, or at least 40, or at least 50, and up to 114 amino acids in length. In certain embodiments, the DAP12 polypeptide comprises or consists of an amino acid sequence of amino acids 1 to 114, 10 to 80, 20 to 80, 30 to 80, 40 to 80, 43 to 80, or 43 to 63 of SEQ ID NO: 108. In certain embodiments, the transmembrane domain of the chimeric receptor (e.g., a CAR-DEL chimeric receptor) comprises a DAP12 polypeptide comprising or consisting of amino acids 43 to 63 of SEQ ID NO: 108. SEQ ID NO: 108 is provided below:

[SEQ ID NO: 108]
MGALEPSWCLLFLPVLLTVGGLSPVQAQSDTFPRCDCSSVSPGVLAGIVL
GDLVLTLLIALAVYSLGRLVSRGQGTAEGTRKQHIAETESPYQELQGQRP
EVYSDLNTQRQYYR

In certain embodiments, the transmembrane domain of the chimeric receptor (e.g., a CAR-DEL chimeric receptor) comprises a transmembrane domain of human 2B4 or a fragment thereof. In certain embodiments, the 2B4 polypeptide comprises or consists of an amino acid sequence that is at least about 85%, about 90%, about 95%, about 96%, about 97%, about 98%, about 99%, or 100% homologous or identical to the amino acid sequence having a UniProt Reference No: Q9BZW8 (SEQ ID No: 109) or a fragment thereof, and/or may optionally comprise up to one or up to two or up to three conservative amino acid substitutions. In certain non-limiting embodiments, the 2B4 polypeptide comprises or consists of an amino acid sequence that is a consecutive portion of SEQ ID NO: 109 which is at least 10, or at least 20, or at least 30, or at least 40, or at least 50, or at least 100, or at least 200, and up to 370 amino acids in length. In certain embodiments, the 2B4 polypeptide comprises or consists of an amino acid sequence of amino acids 1 to 370, 100 to 200, 200 to 250, 215 to 250, or 230 to 268 of SEQ ID NO: 109. In certain embodiments, the transmembrane domain of the chimeric receptor (e.g., a CAR-DEL chimeric receptor) comprises a 2B4 polypeptide comprising or consisting of amino acids 230 to 250 of SEQ ID NO: 109. SEQ ID NO: 109 is provided below:

[SEQ ID NO: 109]
MLGQVVTLILLLLLKVYQGKGCQGSADHVVSISGVPLQLQPNSIQTKVDSIAWKKLLPSQNGFHHILKWE
NGSLPSNTSNDRFSFIVKNLSLLIKAAQQQDSGLYCLEVTSISGKVQTATFQVFVFESLLPDKVEKPRLQ
GQGKILDRGRCQVALSCLVSRDGNVSYAWYRGSKLIQTAGNLTYLDEEVDINGTHTYTCNVSNPVSWESH
TLNLTQDCQNAHQEFRFWPFLVIIVILSALFLGTLACFCVWRRKRKEKQSETSPKEFLTIYEDVKDLKTR
RNHEQEQTFPGGGSTIYSMIQSQSSAPTSQEPAYTLYSLIQPSRKSGSRKRNHSPSENSTIYEVIGKSQP
KAQNPARLSRKELENFDVYS

In certain embodiments, the transmembrane domain of the chimeric receptor (e.g., a CAR-DEL chimeric receptor) comprises a 2B4 polypeptide (e.g., a transmembrane domain of mouse 2B4 or a fragment thereof). In certain embodiments, the 2B4 polypeptide comprises or consists of an amino acid sequence that is at least about 85%, about 90%, about 95%, about 96%, about 97%, about 98%, about 99%, or 100% homologous or identical to the amino acid sequence having a UniProt Reference No: Q07763 (SEQ ID No: 110) or a fragment thereof, and/or may optionally comprise up to one or up to two or up to three conservative amino acid substitutions. In certain embodiments, the 2B4 polypeptide comprises or consists of an amino acid sequence that is a consecutive portion of SEQ ID NO: 110, which is at least 10, or at least 20, or at least 30, or at least 40, or at least 50, or at least 100, or at least 200, and up to 397 amino acids in length. In certain embodiments, the 2B4 polypeptide comprises or consists of an amino acid sequence of amino acids 1 to 397, 100 to 200, 200 to 247, 220 to 247, or 227 to 247 of SEQ ID NO: 110. In certain embodiments, the transmembrane domain of the chimeric receptor (e.g., a CAR-DEL chimeric receptor) comprises a 2B4 polypeptide comprising or consisting of amino acids 227 to 247 of SEQ ID NO: 110. SEQ ID NO: 110 is provided below:

[SEQ ID NO: 110]
MLGQAVLFTTFLLLRAHQGQDCPDSSEEVVGVSGKPVQLRPSNIQTKDVSVQWKKTEQGSHRKIEILNWY
NDGPSWSNVSFSDIYGFDYGDFALSIKSAKLQDSGHYLLEITNTGGKVCNKNFQLLILDHVETPNLKAQW
KPWTNGTCQLFLSCLVTKDDNVSYALYRGSTLISNQRNSTHWENQIDASSLHTYTCNVSNRASWANHTLN
FTHGCQSVPSNFRFLPFGVIIVILVTLFLGAIICFCVWTKKRKQLQFSPKEPLTIYEYVKDSRASRDQQG
CSRASGSPSAVQEDGRGQRELDRRVSEVLEQLPQQTFPGDRGTMYSMIQCKPSDSTSQEKCTVYSVVQPS
RKSGSKKRNQNSSLSCTVYEEVGNPWLKAHNPARLSRRELENFDVYS

In certain embodiments, the transmembrane domain of the chimeric receptor (e.g., a CAR-DEL chimeric receptor) comprises a transmembrane domain of human DNAM1 or a fragment thereof. In certain embodiments, the DNAM1 polypeptide comprises or consists of an amino acid sequence that is at least about 85%, about 90%, about 95%, about 96%, about 97%, about 98%, about 99%, or 100% homologous or identical to the amino acid sequence having a UniProt Reference No: Q15762 (SEQ ID No: 111) or a fragment thereof, and/or may optionally comprise up to one or up to two or up to three conservative amino acid substitutions. In certain non-limiting embodiments, the DNAM1 polypeptide comprises or consists of an amino acid sequence that is a consecutive portion of SEQ ID NO: 111 which is at least 10, or at least 20, or at least 30, or at least 40, or at least 50, or at least 100, or at least 200, or at least 250, and up to 336 amino acids in length. In certain embodiments, the DNAM1 polypeptide comprises or consists of an amino acid sequence of amino acids 1 to 336, 1 to 50, 50 to 100, 100 to 200, 200 to 250, 250 to 275, or 255 to 275 of SEQ ID NO: 111. In certain embodiments, the transmembrane domain of the chimeric receptor (e.g., a CAR-DEL chimeric receptor) comprises a DNAM1 polypeptide comprising or consisting of amino acids 255 to 275 of SEQ ID NO: 111. SEQ ID NO: 111 is provided below:

[SEQ ID NO: 111]
MDYPTLLLALLHVYRALCEEVLWHTSVPFAENMSLECVYPSMGILTQVEWFKIGTQQDSIAIFSPTHGMV
IRKPYAERVYFLNSTMASNNMTLFFRNASEDDVGYYSCSLYTYPQGTWQKVIQVVQSDSFEAAVPSNSHI
VSEPGKNVTLTCQPQMTWPVQAVRWEKIQPRQIDLLTYCNLVHGRNFTSKFPRQIVSNCSHGRWSVIVIP
DVTVSDSGLYRCYLQASAGENETFVMRLTVAEGKTDNQYTLFVAGGTVLLLLFVISITTIIVIFLNRRRR
RERRDLFTESWDTQKAPNNYRSPISTSQPTNQSMDDTREDIYVNYPTFSRRPKTRV

In certain embodiments, the transmembrane domain of the chimeric receptor (e.g., a CAR-DEL chimeric receptor) comprises a DNAM1 polypeptide (e.g., a transmembrane domain of mouse DNAM1 or a fragment thereof). In certain embodiments, the DNAM1 polypeptide comprises or consists of an amino acid sequence that is at least about 85%, about 90%, about 95%, about 96%, about 97%, about 98%, about 99%, or 100% homologous or identical to the amino acid sequence having a UniProt Reference No: Q8K4F0 (SEQ ID No: 112) or a fragment thereof, and/or may optionally comprise up to one or up to two or up to three conservative amino acid substitutions. In certain embodiments, the DNAM1 polypeptide comprises or consists of an amino acid sequence that is a consecutive portion of SEQ ID NO: 112, which is at least 10, or at least 20, or at least 30, or at least 40, or at least 50, or at least 100, or at least 200, and up to 333 amino acids in length. In certain embodiments, the DNAM1 polypeptide comprises or consists of an amino acid sequence of amino acids 1 to 333, 1 to 50, 50 to 100, 100 to 150, 150 to 200, 200 to 250, 250 to 275, or 255 to 275 of SEQ ID NO: 112. In certain embodiments, the transmembrane domain of the chimeric receptor (e.g., a CAR-DEL chimeric receptor) comprises a DNAM1 polypeptide comprising or consisting of amino acids 255 to 275 of SEQ ID NO: 112. SEQ ID NO: 112 is provided below:

[SEQ ID NO: 112]
MAYVTWLLAILHVHKALCEETLWDTTVRLSETMTLECVYPLTHNLTQVEWTKNTGTKTVSIAVYNPNHNM
HIESNYLHRVHFLNSTVGFRNMSLSFYNASEADIGIYSCLFHAFPNGPWEKKIKVVWSDSFEIAAPSDSY
LSAEPGQDVTLTCQLPRTWPVQQVIWEKVQPHQVDILASCNLSQETRYTSKYLRQTRSNCSQGSMKSILI
IPNAMAADSGLYRCRSEAITGKNKSFVIRLIITDGGTNKHFILPIVGGLVSLLLVILIIIIFILYNRKRR
RQVRIPLKEPRDKQSKVATNCRSPTSPIQSTDDEKEDIYVNYPTFSRRPKPRL

In certain non-limiting embodiments, the chimeric receptor (e.g., a CAR-DEL chimeric receptor) further comprises a spacer region that links the extracellular antigen-binding domain to the transmembrane domain. The spacer region can be flexible enough to allow the antigen binding domain to orient in different directions to facilitate antigen recognition while preserving the properties of the chimeric receptor (e.g., a CAR-DEL chimeric receptor).

In certain embodiments, the hinge/spacer region of the chimeric receptor (e.g., a CAR-DEL chimeric receptor) comprises a native or modified hinge region of CD8 or a fragment thereof, a native or modified hinge region of CD28 or a fragment thereof, a native or modified hinge region of CD35 or a fragment thereof, a native or modified hinge region of CD4 or a fragment thereof, a native or modified hinge region of 4-1BB or a fragment thereof, a native or modified hinge region of OX40 or a fragment thereof, a native or modified hinge region of CD84 or a fragment thereof, a native or modified hinge region of CD166 or a fragment thereof, a native or modified hinge region of CD8a or a fragment thereof, a native or modified hinge region of CD8b or a fragment thereof, a native or modified hinge region of ICOS or a fragment thereof, a native or modified hinge region of ICAM-1 or a fragment thereof, a native or modified hinge region of CTLA-4 or a fragment thereof, a native or modified hinge region of CD27 or a fragment thereof, a native or modified hinge region of CD40 or a fragment thereof, a native or modified hinge region of NKGD2 or a fragment thereof, a native or modified hinge region of PD-1 or a fragment thereof, a native or modified hinge region of LAG-3 or a fragment thereof, a native or modified hinge region of 2B4 or a fragment thereof, a native or modified hinge region of BTLA or a fragment thereof, a native or modified hinge region of DAP12 or a fragment thereof, a native or modified hinge region of DAP10 or a fragment thereof, a native or modified hinge region of CD16 or a fragment thereof, a native or modified hinge region of DNAM1 or a fragment thereof, a synthetic polypeptide (not based on a protein associated with the immune response), or a combination thereof.

The hinge/spacer region can be the hinge region from IgG1, or the CH₂CH₃ region of immunoglobulin and portions of CD3, a portion of a CD8 polypeptide (e.g., a portion of SEQ ID NO: 38 or 39), a portion of a CD28 polypeptide (e.g., a portion of SEQ ID NO: 40 or 42), a variation of any of the foregoing which is at least about 80%, at least about 85%, at least about 90%, at least about 95%, or at least about 100% homologous or identical thereto, or a synthetic spacer sequence.

In certain embodiments, the hinge/spacer region of the chimeric receptor (e.g., a CAR-DEL chimeric receptor) comprises a CD28 polypeptide comprising or consisting of amino acids 114 to 152 of SEQ ID NO: 40. In certain embodiments, the hinge/spacer region of the chimeric receptor (e.g., a CAR-DEL chimeric receptor) comprises or consists of an amino acid sequence that is at least about 80%, at least about 85%, at least about 90%, at least about 95%, or at least about 99% identical to the amino acid sequence set forth in SEQ ID NO: 20. In certain embodiments, the hinge/spacer region of the CAR comprises or consists of the amino acid sequence set forth in SEQ ID NO: 20. SEQ ID NO: 20 is provided below:

[SEQ ID NO: 20]
IEVMYPPPYLDNEKSNGTIIHVKGKHLCPSPLFPGPSKP

### 2.1.4. Intracellular Polypeptide

In certain embodiments, the chimeric receptor (e.g., a CAR-DEL chimeric receptor) comprises an intracellular polypeptide. In certain embodiments, the intracellular polypeptide does not deliver an activation signal to a cell. In certain embodiments, the intracellular polypeptide does not deliver a co-stimulatory signal to a cell. In certain embodiments, the intracellular polypeptide does not deliver an activation signal to a cell and a co-stimulatory signal to a cell.

In certain embodiments, the intracellular polypeptide of the chimeric receptor (e.g., a CAR-DEL chimeric receptor) can comprise a native or modified intracellular polypeptide of CD8 or a fragment thereof, a native or modified intracellular polypeptide of CD28 or a fragment thereof, a native or modified intracellular polypeptide of CD4 or a fragment thereof, a native or modified intracellular polypeptide of 4-1BB or a fragment thereof, a native or modified intracellular polypeptide of OX40 or a fragment thereof, a native or modified intracellular polypeptide of ICOS or a fragment thereof, a native or modified intracellular polypeptide of CD84 or a fragment thereof, a native or modified intracellular polypeptide of CD166 or a fragment thereof, a native or modified intracellular polypeptide of CD8a or a fragment thereof, a native or modified intracellular polypeptide of CD8b or a fragment thereof, a native or modified intracellular polypeptide of ICAM-1 or a fragment thereof, a native or modified intracellular polypeptide of CTLA-4 or a fragment thereof, a native or modified intracellular polypeptide of CD27 or a fragment thereof, a native or modified intracellular polypeptide of CD40 or a fragment thereof, a native or modified intracellular polypeptide of NKGD2 or a fragment thereof, a native or modified intracellular polypeptide of PD-1 or a fragment thereof, a native or modified intracellular polypeptide of LAG-3 or a fragment thereof, a native or modified intracellular polypeptide of 2B4 or a fragment thereof, a native or modified intracellular polypeptide of BTLA or a fragment thereof a native or modified intracellular polypeptide of DAP12 or a fragment thereof, a native or modified intracellular polypeptide of DAP10 or a fragment thereof, a native or modified intracellular polypeptide of CD16 or a fragment thereof, a native or modified intracellular polypeptide of CD266 or a fragment thereof, or a combination thereof.

In certain embodiments, the intracellular polypeptide of the chimeric receptor (e.g., a CAR-DEL chimeric receptor) comprises an intracellular polypeptide of human CD28 or a fragment thereof. In certain embodiments, the CD28 intracellular polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 40 or a fragment thereof, and/or may optionally comprise up to one or up to two or up to three conservative amino acid substitutions. In certain embodiments, the CD28 intracellular polypeptide comprises or consists of an amino acid sequence that is a consecutive portion of SEQ ID NO: 40, which is at least 20, or at least 30, or at least 40, or at least 50, and up to 190 amino acids in length. In certain embodiments, the CD28 polypeptide comprises or consists of an amino acid sequence of amino acids 1 to 190, 1 to 50, 50 to 100, 100 to 150, 114 to 190, 150 to 190, or 180 to 190, of SEQ ID NO: 40. In certain embodiments, the intracellular polypeptide of the chimeric receptor (e.g., a CAR-DEL chimeric receptor) comprises a CD28 intracellular polypeptide comprising or consisting of an amino acid sequence of amino acids 180 to 190 of SEQ ID NO: 40. In certain embodiments, the intracellular domain of the chimeric receptor comprises a CD28 polypeptide comprising or consisting of the amino acid sequence set for in SEQ ID NO: 16. SEQ ID NO: 16 is provided below:

[SEQ ID NO: 16]
RSKRSRLLHSD

An exemplary nucleotide sequence encoding amino acids 180 to 190 of SEQ ID NO: 40 is set forth in SEQ ID NO: 46, which is provided below.

[SEQ ID NO: 46]
AGGAGTAAGAGGAGCAGGCTCCTGCACAGTGAC

### 2.1.4.1. Exemplary Chimeric Receptors

In certain embodiments, the chimeric receptor (e.g., a CAR-DEL chimeric receptor) is a CD33-targeted chimeric receptor. In certain embodiments, the chimeric receptor comprises (a) an extracellular antigen-binding domain comprising (i) a V_{H} that comprises a CDR1 comprising the amino acid sequence set forth in SEQ ID NO: 28, a CDR2 comprising the amino acid sequence set forth in SEQ ID NO: 29, and a CDR3 comprising the amino acid sequence set forth in SEQ ID NO: 30, and (ii) a V_{L} that comprises a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33; (b) a transmembrane domain comprising a CD28 polypeptide (e.g., a transmembrane domain of human CD28 or a fragment thereof), and (c) an intracellular polypeptide comprising a CD28 polypeptide. In certain embodiments, the transmembrane domain comprises a CD28 polypeptide that comprises amino acids 153 to 179 of SEQ ID NO: 40. In certain embodiments, the intracellular polypeptide comprises a CD28 polypeptide comprising amino acids 180 to 190 of SEQ ID NO: 40. In certain embodiments, the CAR is designed as “3p14mtDEL.” In certain embodiments, the CAR-DEL comprises the amino acid sequence set forth in SEQ ID NO: 21, which is provided below.

[SEQ ID NO: 21]
EVQLLESGGGLVQPGGSLRLSCAASGFTFSTYAMSWVRQAPGKGLEWVSAISGRGGSTYYTDSV
KGRFTISRDNSKNTVSLOMNSLRAEDTAVYYCAGRGDYYYYYGMDVWGQGTTVTVSAGGGGSGG
GGSGGGGSDIVMTQSPLSSPVTLGQPASFSCRSSQSLVYSDGNTYLSWLQQRPGQPPRLLIYKI
SNRFSGVPDRFSGSGAGTDFTLKISRVEAEDVGVYYCMQSTQFPHTFGQGTKLEIKEQKLISEE
DLAAAIEVMYPPPYLDNEKSNGTIIHVKGKHLCPSPLFPGPSKPFWVLVVVGGVLACYSLLVTV
AFIIFWVRSKRSRLLHSD

In certain embodiments, the CAR-DEL chimeric receptor comprises the amino acid sequence set forth in SEQ ID NO: 22, which is provided below.

[SEQ ID NO: 22]
EVQLLESGGGLVQPGGSLRLSCAASGFTESTYAMSWVRQAPGKGLEWVSAISGRGGSTYYTDSV
KGRFTISRDNSKNTVSLQMNSLRAEDTAVYYCAGRGDYYYYYGMDVWGQGTTVTVSAGGGGSGG
GGSGGGGSDIVMTQSPLSSPVTLGQPASFSCRSSQSLVYSDGNTYLSWLQQRPGQPPRLLIYKI
SNRFSGVPDRESGSGAGTDFTLKISRVEAEDVGVYYCMQSTQFPHTFGQGTKLEIKIEVMYPPP
YLDNEKSNGTIIHVKGKHLCPSPLFPGPSKPFWVLVVVGGVLACYSLLVTVAFIIFWVRSKRSR
LLHSD

### 2.1.5. Nucleic Acid Molecules Encoding the Chimeric Receptors

The presently disclosed subject matter further provides nucleic acid molecules encoding the chimeric receptor (e.g., a CAR-DEL chimeric receptor) disclosed herein. In addition, the presently disclosed subject matter provides vectors comprising the nucleic acid molecules described herein. The vectors can be viral vectors or non-viral vectors. In certain embodiments, the vector is a viral vector. In certain embodiments, the viral vector is a retroviral vector, e.g., a gammaretroviral vector, or a lentiviral vector.

### 2.1.6. Delivery of the Chimeric Receptors

In certain embodiments, the chimeric receptor (e.g., a CAR-DEL chimeric receptor) or a polynucleotide encoding the same is delivered to the cell by a viral method. In certain embodiments, the viral method comprises a viral vector. In certain embodiments, the viral vector is a retroviral vector (e.g., a gammaretroviral vector or a lentiviral vector). Other viral vectors include adenoviral vectors, adeno-associated viral vectors, vaccinia viruses, bovine papilloma viruses, and herpes viruses (e.g., such as Epstein-Barr Virus).

In certain embodiments, the chimeric receptor (e.g., a CAR-DEL chimeric receptor) or a polynucleotide encoding the same is delivered to the cell by a non-viral method. Any targeted genome editing methods can also be used to deliver the chimeric receptor to the cell. In certain embodiments, the chimeric receptor is delivered to the cell by a method comprising homologous recombination, a Zinc finger nuclease, a meganuclease, a Transcription activator-like effector nuclease (TALEN), a Clustered regularly-interspaced short palindromic repeats (CRISPR) system, or a combination thereof. In certain embodiments, a CRISPR system is used to deliver the chimeric receptor to the cell.

In certain embodiments, the cell is a NK cell, and the chimeric receptor (e.g., CAR-DEL) or a polynucleotide encoding the same is integrated at a locus within the genome of the NK cell.

Additionally or alternatively, the chimeric receptor (e.g., a CAR-DEL chimeric receptor) or a polynucleotide encoding the same is integrated at a genomic safe harbor within the genome of the NK cell. Further information on genomic safe harbors and on methods for identifying the same can be found in International Patent Publications No. 2021/055592 and No. 2021/055616, the contents of each of which are incorporated by reference in their entirety.

### 2.2. Cytokines

In certain embodiments, the cells disclosed herein further comprise a cytokine. In certain embodiments, the cells disclosed herein further comprise a first cytokine. In certain embodiments, cells disclosed herein further comprise a second cytokine. In certain embodiments, the cytokine can be a chemokine, an interferon, an interleukin, a lymphokine, or a tumor necrosis factor. In certain embodiments, the cytokine is an interleukin. Non-limiting examples of interleukin include IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, and IL-36.

In certain embodiments, the cytokine is an IL-15 polypeptide or a functional fragment thereof. In certain embodiments, the IL-15 polypeptide is a human IL-15 polypeptide. In certain embodiments, the IL-15 polypeptide comprises or consists of an amino acid sequence that is at least about 80%, about 85%, about 90%, about 95%, about 96%, about 97%, about 98%, about 99%, or about 100% identical to the amino acid sequence having a UniProt Reference No: P40933 (SEQ ID NO: 113) or a functional fragment thereof. In certain embodiments, the IL-15 polypeptide can optionally comprise up to one or up to two or up to three conservative amino acid substitutions. In certain embodiments, the IL-15 polypeptide comprises or consists of an amino acid sequence that is a consecutive portion of SEQ ID NO: 113, which is at least about 20, or at least about 30, or at least about 40, at least about 50, at least about 100, at least about 150, and up to about 162 amino acids in length. In certain embodiments, the IL-15 polypeptide comprises or consists of the amino acid sequence of amino acids 1 to 162, 30 to 162, or 49 to 162 of SEQ ID NO: 113. In certain embodiments, the IL-15 polypeptide comprises or consists of the amino acid sequence of amino acids 49 to 162 of SEQ ID NO: 113. SEQ ID NO: 113 is provided below.

[SEQ ID NO: 113]
MRISKPHLRSISIQCYLCLLLNSHELTEAGIHVFILGCFSAGLPKTEAN
WVNVISDLKKIEDLIQSMHIDATLYTESDVHPSCKVTAMKCFLLELQVI
SLESGDASIHDTVENLIILANNSLSSNGNVTESGCKECEELEEKNIKEF
LQSFVHIVQMFINTS

In certain embodiments, the IL-15 polypeptide comprises the amino acid sequence set forth in SEQ ID NO: 114. In certain embodiments, the IL-15 polypeptide consists of the amino acid sequence set forth in SEQ ID NO: 114. SEQ ID NO: 114 is provided below.

[SEQ ID NO: 114]
GIHVFILGCFSAGLPKTEANWVNVISDLKKIEDLIQSMHIDATLYTESD
VHPSCKVTAMKCELLELQVISLESGDASIHDTVENLIILANNSLSSNGN
VTESGCKECEELEEKNIKEFLQSFVHIVQMFINTS

In certain embodiments, the IL-15 polypeptide comprises the amino acid sequence set forth in SEQ ID NO: 115. In certain embodiments, the IL-15 polypeptide consists of the amino acid sequence set forth in SEQ ID NO: 115. SEQ ID NO: 115 is provided below.

[SEQ ID NO: 115]
NWVNVISDLKKIEDLIQSMHIDATLYTESDVHPSCKVTAMKCFLLELQV
ISLESGDASIHDTVENLIILANNSLSSNGNVTESGCKECEELEEKNIKE
FLQSFVHIVQMFINTS

In certain embodiments, the IL-15 polypeptide or a functional fragment thereof retains at least about 80%, at least about 85%, at least about 90%, at least about 95%, or at least about 100% of the primary function of a native IL-15 polypeptide. For example, but without any limitation, the IL-15 polypeptide or a functional fragment thereof retains the ability to bind to and interact with IL-15 receptor.

In certain embodiments, the cytokine is an IL-33 polypeptide or a functional fragment thereof. In certain embodiments, the IL-33 polypeptide is a human IL-33 polypeptide. In certain embodiments, the IL-33 polypeptide comprises or consists of an amino acid sequence that is at least about 80%, about 85%, about 90%, about 95%, about 96%, about 97%, about 98%, about 99%, or about 100% identical to the amino acid sequence having a UniProt Reference No: 095760 (SEQ ID NO: 116) or a functional fragment thereof. In certain embodiments, the IL-33 polypeptide can optionally comprise up to one or up to two or up to three conservative amino acid substitutions. In certain embodiments, the IL-33 polypeptide comprises or consists of an amino acid sequence that is a consecutive portion of SEQ ID NO: 116, which is at least about 20, or at least about 30, or at least about 40, at least about 50, at least about 100, at least about 150, at least about 200, at least about 250, and up to about 270 amino acids in length. In certain embodiments, the IL-33 polypeptide comprises or consists of the amino acid sequence of amino acids 1 to 270, 95 to 270, 99 to 270, or 109 to 270 of SEQ ID NO: 116. In certain embodiments, the IL-33 polypeptide comprises or consists of the amino acid sequence of amino acids 1 to 270 of SEQ ID NO: 116. SEQ ID NO: 116 is provided below.

[SEQ ID NO: 116]
MKPKMKYSTNKISTAKWKNTASKALCFKLGKSQQKAKEVCPMYFMKLRSGLMIKKEACYFRRETTKRPSL
KTGRKHKRHLVLAACQQQSTVECFAFGISGVQKYTRALHDSSITGISPITEYLASLSTYNDQSITFALED
ESYEIYVEDLKKDEKKDKVLLSYYESQHPSNESGDGVDGKMLMVTLSPTKDFWLHANNKEHSVELHKCEK
PLPDQAFFVLHNMHSNCVSFECKTDPGVFIGVKDNHLALIKVDSSENLCTENILEKLSET

In certain embodiments, the IL-33 polypeptide comprises the amino acid sequence set forth in SEQ ID NO: 117. In certain embodiments, the IL-33 polypeptide consists of the amino acid sequence set forth in SEQ ID NO: 117. SEQ ID NO: 117 is provided below.

[SEQ ID NO: 117]
AFGISGVQKYTRALHDSSITGISPITEYLASLSTYNDQSITFALEDESY
EIYVEDLKKDEKKDKVLLSYYESQHPSNESGDGVDGKMLMVTLSPTKDE
WLHANNKEHSVELHKCEKPLPDQAFFVLHNMHSNCVSFECKTDPGVFIG
VKDNHLALIKVDSSENLCTENILEKLSET

In certain embodiments, the IL-33 polypeptide comprises the amino acid sequence set forth in SEQ ID NO: 118. In certain embodiments, the IL-33 polypeptide consists of the amino acid sequence set forth in SEQ ID NO: 118. SEQ ID NO: 118 is provided below.

[SEQ ID NO: 118]
SGVQKYTRALHDSSITGISPITEYLASLSTYNDQSITFALEDESYEIYV
EDLKKDEKKDKVLLSYYESQHPSNESGDGVDGKMLMVTLSPTKDFWLHA
NNKEHSVELHKCEKPLPDQAFFVLHNMHSNCVSFECKTDPGVFIGVKDN
HLALIKVDSSENLCTENILFKLSET

In certain embodiments, the IL-33 polypeptide comprises the amino acid sequence set forth in SEQ ID NO: 119. In certain embodiments, the IL-33 polypeptide consists of the amino acid sequence set forth in SEQ ID NO: 119. SEQ ID NO: 119 is provided below.

[SEQ ID NO: 119]
HDSSITGISPITEYLASLSTYNDQSITFALEDESYEIYVEDLKKDEKKD
KVLLSYYESQHPSNESGDGVDGKMLMVTLSPTKDFWLHANNKEHSVELH
KCEKPLPDQAFFVLHNMHSNCVSFECKTDPGVFIGVKDNHLALIKVDSS
ENLCTENILFKLSET

In certain embodiments, the IL-33 polypeptide comprises the amino acid sequence set forth in SEQ ID NO: 120. In certain embodiments, the IL-33 polypeptide consists of the amino acid sequence set forth in SEQ ID NO: 120. SEQ ID NO: 120 is provided below.

[SEQ ID NO: 120]
SITGISPITEYLASLSTYNDQSITFALEDESYEIYVEDLKKDEKKDKVL
LSYYESQHPSNESGDGVDGKMLMVTLSPTKDFWLHANNKEHSVELHKCE
KPLPDQAFFVLHNMHSNCVSFECKTDPGVFIGVKDNHLALIKVDSSENL
CTENILFKLSET

In certain embodiments, the IL-33 polypeptide or a functional fragment thereof retains at least about 80%, at least about 85%, at least about 90%, at least about 95%, or at least about 100% of the primary function of a native IL-33 polypeptide. For example, but without any limitation, the IL-33 polypeptide or a functional fragment thereof retains the ability to bind to and interact with IL-33 receptor.

In certain embodiments, the cytokine is an IL-18 polypeptide or a functional fragment thereof. In certain embodiments, the IL-18 polypeptide is a human IL-18 polypeptide. In certain embodiments, the IL-18 polypeptide comprises or consists of an amino acid sequence that is at least about 80%, about 85%, about 90%, about 95%, about 96%, about 97%, about 98%, about 99%, or about 100% identical to the amino acid sequence having a UniProt Reference No: Q14116 (SEQ ID NO: 121) or a functional fragment thereof. In certain embodiments, the IL-18 polypeptide can optionally comprise up to one or up to two or up to three conservative amino acid substitutions. In certain embodiments, the IL-18 polypeptide comprises or consists of an amino acid sequence that is a consecutive portion of SEQ ID NO: 121, which is at least about 20, or at least about 30, or at least about 40, at least about 50, at least about 100, at least about 150, and up to about 193 amino acids in length. In certain embodiments, the IL-18 polypeptide comprises or consists of the amino acid sequence of amino acids 1 to 193 or 37 to 193 of SEQ ID NO: 121. In certain embodiments, the IL-18 polypeptide comprises or consists of the amino acid sequence of amino acids 1 to 193 of SEQ ID NO: 121. SEQ ID NO: 121 is provided below.

[SEQ ID NO: 121]
MAAEPVEDNCINFVAMKFIDNTLYFIAEDDENLESDYFGKLESKLSVIR
NLNDQVLFIDQGNRPLFEDMTDSDCRDNAPRTIFIISMYKDSQPRGMAV
TISVKCEKISTLSCENKIISFKEMNPPDNIKDTKSDIIFFQRSVPGHDN
KMQFESSSYEGYFLACEKERDLFKLILKKEDELGDRSIMETVQNED

In certain embodiments, the IL-18 polypeptide comprises the amino acid sequence set forth in SEQ ID NO: 122. In certain embodiments, the IL-18 polypeptide consists of the amino acid sequence set forth in SEQ ID NO: 122. SEQ ID NO: 122 is provided below.

[SEQ ID NO: 122]
YFGKLESKLSVIRNLNDQVLFIDQGNRPLFEDMTDSDCRDNAPRTIFII
SMYKDSQPRGMAVTISVKCEKISTLSCENKIISFKEMNPPDNIKDTKSD
IIFFQRSVPGHDNKMQFESSSYEGYFLACEKERDLFKLILKKEDELGDR
SIMFTVQNED

In certain embodiments, the IL-18 polypeptide or a functional fragment thereof retains at least about 80%, at least about 85%, at least about 90%, at least about 95%, or at least about 100% of the primary function of a native IL-18 polypeptide. For example, but without any limitation, the IL-18 polypeptide or a functional fragment thereof retains the ability to bind to and interact with IL-18 receptor.

In certain embodiments, the cytokine is an IL-12 polypeptide or a functional fragment thereof. In certain embodiments, the IL-12 polypeptide is a human IL-12 polypeptide. In certain embodiments, the IL-12 polypeptide comprises or consists of an amino acid sequence that is at least about 80%, about 85%, about 90%, about 95%, about 96%, about 97%, about 98%, about 99%, or about 100% identical to the amino acid sequence having a UniProt Reference No: P29459 (SEQ ID NO: 123) or a functional fragment thereof. In certain embodiments, the IL-12 polypeptide can optionally comprise up to one or up to two or up to three conservative amino acid substitutions. In certain embodiments, the IL-12 polypeptide comprises or consists of an amino acid sequence that is a consecutive portion of SEQ ID NO: 123, which is at least about 20, or at least about 30, or at least about 40, at least about 50, at least about 100, at least about 150, at least about 200, and up to about 219 amino acids in length. In certain embodiments, the IL-12 polypeptide comprises or consists of the amino acid sequence of amino acids 1 to 219 or 23 to 219 of SEQ ID NO: 123. In certain embodiments, the IL-12 polypeptide comprises or consists of the amino acid sequence of amino acids 1 to 219 of SEQ ID NO: 123. SEQ ID NO: 123 is provided below.

[SEQ ID NO: 123]
MCPARSLLLVATLVLLDHLSLARNLPVATPDPGMFPCLHHSQNLL
RAVSNMLQKARQTLEFYPCTSEEIDHEDITKDKTSTVEACLPLEL
TKNESCLNSRETSFITNGSCLASRKTSEMMALCLSSIYEDLKMYQ
VEFKTMNAKLLMDPKRQIFLDQNMLAVIDELMQALNENSETVPQK
SSLEEPDFYKTKIKLCILLHAFRIRAVTIDRVMSYLNAS

In certain embodiments, the IL-12 polypeptide comprises the amino acid sequence set forth in SEQ ID NO: 124. In certain embodiments, the IL-12 polypeptide consists of the amino acid sequence set forth in SEQ ID NO: 124. SEQ ID NO: 124 is provided below.

[SEQ ID NO: 124]
RNLPVATPDPGMFPCLHHSQNLLRAVSNMLQKARQTLEFYPCTSE
EIDHEDITKDKTSTVEACLPLELTKNESCLNSRETSFITNGSCLA
SRKTSEMMALCLSSIYEDLKMYQVEFKTMNAKLLMDPKRQIFLDQ
NMLAVIDELMQALNENSETVPQKSSLEEPDFYKTKIKLCILLHAF
RIRAVTIDRVMSYLNAS

In certain embodiments, the IL-12 polypeptide or a functional fragment thereof retains at least about 80%, at least about 85%, at least about 90%, at least about 95%, or at least about 100% of the primary function of a native IL-12 polypeptide. For example, but without any limitation, the IL-12 polypeptide or a functional fragment thereof retains the ability to bind to and interact with IL-12 receptor.

In certain embodiments, the cytokine is an IL-2 polypeptide or a functional fragment thereof. In certain embodiments, the IL-2 polypeptide is a human IL-2 polypeptide. In certain embodiments, the IL-2 polypeptide comprises or consists of an amino acid sequence that is at least about 80%, about 85%, about 90%, about 95%, about 96%, about 97%, about 98%, about 99%, or about 100% identical to the amino acid sequence having a UniProt Reference No: P60568 (SEQ ID NO: 25) or a functional fragment thereof. In certain embodiments, the IL-2 polypeptide can optionally comprise up to one or up to two or up to three conservative amino acid substitutions. In certain embodiments, the IL-2 polypeptide comprises or consists of an amino acid sequence that is a consecutive portion of SEQ ID NO: 25, which is at least about 20, or at least about 30, or at least about 40, at least about 50, at least about 100, at least about 150, and up to about 153 amino acids in length. In certain embodiments, the IL-2 polypeptide comprises or consists of the amino acid sequence of amino acids 1 to 153 or 21 to 153 of SEQ ID NO: 25. In certain embodiments, the IL-2 polypeptide comprises or consists of the amino acid sequence of amino acids 1 to 153 of SEQ ID NO: 25. SEQ ID NO: 25 is provided below.

[SEQ ID NO: 25]
MYRMQLLSCIALSLALVINSAPTSSSTKKTQLQLEHLLLDLQMIL
NGINNYKNPKLTRMLTFKFYMPKKATELKHLQCLEEELKPLEEVL
NLAQSKNFHLRPRDLISNINVIVLELKGSETTEMCEYADETATIV
EFLNRWITFCQSIISTLT

In certain embodiments, the IL-2 polypeptide or a functional fragment thereof retains at least about 80%, at least about 85%, at least about 90%, at least about 95%, or at least about 100% of the primary function of a native IL-2 polypeptide. For example, but without any limitation, the IL-2 polypeptide or a functional fragment thereof retains the ability to bind to and interact with IL-2 receptor.

In certain embodiments, the cytokine is an IL-4 polypeptide or a functional fragment thereof. In certain embodiments, the IL-4 polypeptide is a human IL-4 polypeptide. In certain embodiments, the IL-4 polypeptide comprises or consists of an amino acid sequence that is at least about 80%, about 85%, about 90%, about 95%, about 96%, about 97%, about 98%, about 99%, or about 100% identical to the amino acid sequence having a UniProt Reference No: P05112 (SEQ ID NO: 130) or a functional fragment thereof. In certain embodiments, the IL-4 polypeptide can optionally comprise up to one or up to two or up to three conservative amino acid substitutions. In certain embodiments, the IL-4 polypeptide comprises or consists of an amino acid sequence that is a consecutive portion of SEQ ID NO: 130, which is at least about 20, or at least about 30, or at least about 40, at least about 50, at least about 100, at least about 150, and up to about 153 amino acids in length. In certain embodiments, the IL-4 polypeptide comprises or consists of the amino acid sequence of amino acids 1 to 153 or 25 to 153 of SEQ ID NO: 130. In certain embodiments, the IL-4 polypeptide comprises or consists of the amino acid sequence of amino acids 1 to 153 of SEQ ID NO: 130. SEQ ID NO: 130 is provided below.

[SEQ ID NO: 130]
MGLTSQLLPPLFFLLACAGNFVHGHKCDITLQEIIKTLNSLTEQK
TLCTELTVTDIFAASKNTTEKETFCRAATVLRQFYSHHEKDTRCL
GATAQQFHRHKQLIRFLKRLDRNLWGLAGLNSCPVKEANQSTLEN
FLERLKTIMREKYSKCSS

In certain embodiments, the IL-4 polypeptide or a functional fragment thereof retains at least about 80%, at least about 85%, at least about 90%, at least about 95%, or at least about 100% of the primary function of a native IL-4 polypeptide. For example, but without any limitation, the IL-4 polypeptide or a functional fragment thereof retains the ability to bind to and interact with IL-4 receptor.

In certain embodiments, the cytokine is an IL-7 polypeptide or a functional fragment thereof. In certain embodiments, the IL-7 polypeptide is a human IL-7 polypeptide. In certain embodiments, the IL-7 polypeptide comprises or consists of an amino acid sequence that is at least about 80%, about 85%, about 90%, about 95%, about 96%, about 97%, about 98%, about 99%, or about 100% identical to the amino acid sequence having a UniProt Reference No: P13232 (SEQ ID NO: 131) or a functional fragment thereof. In certain embodiments, the IL-7 polypeptide can optionally comprise up to one or up to two or up to three conservative amino acid substitutions. In certain embodiments, the IL-7 polypeptide comprises or consists of an amino acid sequence that is a consecutive portion of SEQ ID NO: 131, which is at least about 20, or at least about 30, or at least about 40, at least about 50, at least about 100, at least about 150, and up to about 177 amino acids in length. In certain embodiments, the IL-7 polypeptide comprises or consists of the amino acid sequence of amino acids 1 to 177 or 26 to 177 of SEQ ID NO: 131. In certain embodiments, the IL-7 polypeptide comprises or consists of the amino acid sequence of amino acids 1 to 177 of SEQ ID NO: 131. SEQ ID NO: 131 is provided below.

[SEQ ID NO: 131]
MFHVSFRYIFGLPPLILVLLPVASSDCDIEGKDGKQYESVLMVSI
DQLLDSMKEIGSNCLNNEFNFFKRHICDANKEGMFLFRAARKLRQ
FLKMNSTGDFDLHLLKVSEGTTILLNCTGQVKGRKPAALGEAQPT
KSLEENKSLKEQKKLNDLCFLKRLLQEIKTCWNKILMGTKEH

In certain embodiments, the IL-7 polypeptide or a functional fragment thereof retains at least about 80%, at least about 85%, at least about 90%, at least about 95%, or at least about 100% of the primary function of a native IL-7 polypeptide. For example, but without any limitation, the IL-7 polypeptide or a functional fragment thereof retains the ability to bind to and interact with IL-7 receptor.

In certain embodiments, the cytokine is an IL-9 polypeptide or a functional fragment thereof. In certain embodiments, the IL-9 polypeptide is a human IL-9 polypeptide. In certain embodiments, the IL-9 polypeptide comprises or consists of an amino acid sequence that is at least about 80%, about 85%, about 90%, about 95%, about 96%, about 97%, about 98%, about 99%, or about 100% identical to the amino acid sequence having a UniProt Reference No: P15248 (SEQ ID NO: 132) or a functional fragment thereof. In certain embodiments, the IL-9 polypeptide can optionally comprise up to one or up to two or up to three conservative amino acid substitutions. In certain embodiments, the IL-9 polypeptide comprises or consists of an amino acid sequence that is a consecutive portion of SEQ ID NO: 132, which is at least about 20, or at least about 30, or at least about 40, at least about 50, at least about 100, at least about 120, and up to about 144 amino acids in length. In certain embodiments, the IL-9 polypeptide comprises or consists of the amino acid sequence of amino acids 1 to 144 or 19 to 144 of SEQ ID NO: 132. In certain embodiments, the IL-9 polypeptide comprises or consists of the amino acid sequence of amino acids 1 to 144 of SEQ ID NO: 132. SEQ ID NO: 132 is provided below.

[SEQ ID NO: 132]
MLLAMVLTSALLLCSVAGQGCPTLAGILDINFLINKMQEDPASKC
HCSANVTSCLCLGIPSDNCTRPCFSERLSQMTNTTMQTRYPLIFS
RVKKSVEVLKNNKCPYFSCEQPCNQTTAGNALTFLKSLLEIFQKE
KMRGMRGKI

In certain embodiments, the IL-9 polypeptide or a functional fragment thereof retains at least about 80%, at least about 85%, at least about 90%, at least about 95%, or at least about 100% of the primary function of a native IL-9 polypeptide. For example, but without any limitation, the IL-9 polypeptide or a functional fragment thereof retains the ability to bind to and interact with IL-9 receptor.

In certain embodiments, the cytokine is an IL-21 polypeptide or a functional fragment thereof. In certain embodiments, the IL-21 polypeptide is a human IL-21 polypeptide. In certain embodiments, the IL-21 polypeptide comprises or consists of an amino acid sequence that is at least about 80%, about 85%, about 90%, about 95%, about 96%, about 97%, about 98%, about 99%, or about 100% identical to the amino acid sequence having a UniProt Reference No: Q9HBE4 (SEQ ID NO: 133) or a functional fragment thereof. In certain embodiments, the IL-9 polypeptide can optionally comprise up to one or up to two or up to three conservative amino acid substitutions. In certain embodiments, the IL-21 polypeptide comprises or consists of an amino acid sequence that is a consecutive portion of SEQ ID NO: 133, which is at least about 20, or at least about 30, or at least about 40, at least about 50, at least about 100, at least about 150, and up to about 162 amino acids in length. In certain embodiments, the IL-21 polypeptide comprises or consists of the amino acid sequence of amino acids 1 to 162 or 25 to 162 of SEQ ID NO: 133. In certain embodiments, the IL-21 polypeptide comprises or consists of the amino acid sequence of amino acids 1 to 162 of SEQ ID NO: 133. SEQ ID NO: 133 is provided below.

[SEQ ID NO: 133]
MRSSPGNMERIVICLMVIFLGTLVHKSSSQGQDRHMIRMRQLIDI
VDQLKNYVNDLVPEFLPAPEDVETNCEWSAFSCFQKAQLKSANTG
NNERIINVSIKKLKRKPPSTNAGRRQKHRLTCPSCDSYEKKPPKE
FLERFKSLLQKMIHQHLSSRTHGSEDS

In certain embodiments, the IL-21 polypeptide or a functional fragment thereof retains at least about 80%, at least about 85%, at least about 90%, at least about 95%, or at least about 100% of the primary function of a native IL-21 polypeptide. For example, but without any limitation, the IL-21 polypeptide or a functional fragment thereof retains the ability to bind to and interact with IL-21 receptor.

Additional information on cytokines encompassed by the presently disclosed subject matter can be found in Zhou et al., Frontiers in Immunology 14 (2023): 1090311, which is incorporated by reference in its entirety.

### 2.3. Cytokine Receptors

In certain embodiments, the cells disclosed herein further comprise a cytokine receptor. In certain embodiments, the cells disclosed herein further comprise a first cytokine receptor. In certain embodiments, cells disclosed herein further comprise a second cytokine receptor.

In certain embodiments, the cytokine receptor is an IL-15 receptor (IL-15R) polypeptide. In certain embodiments, the IL-15R polypeptide is a human IL-15R polypeptide. In certain embodiments, the IL-15R polypeptide comprises or consists of an amino acid sequence that is at least about 80%, about 85%, about 90%, about 95%, about 96%, about 97%, about 98%, about 99%, or about 100% identical to the amino acid sequence having a UniProt Reference No: Q13261 (SEQ ID NO: 26) or a functional fragment thereof. In certain embodiments, the IL-15R polypeptide can optionally comprise up to one or up to two or up to three conservative amino acid substitutions. In certain embodiments, the IL-15R polypeptide comprises or consists of an amino acid sequence that is a consecutive portion of SEQ ID NO: 26, which is at least about 20, or at least about 30, or at least about 40, at least about 50, at least about 100, at least about 150, and up to about 267 amino acids in length. In certain embodiments, the IL-15R polypeptide comprises or consists of the amino acid sequence of amino acids 1 to 267, 31 to 267, or 51 to 267 of SEQ ID NO: 26. In certain embodiments, the IL-15R polypeptide comprises or consists of the amino acid sequence of amino acids 1 to 267 of SEQ ID NO: 26. SEQ ID NO: 26 is provided below.

[SEQ ID NO: 26]
MAPRRARGCRTLGLPALLLLLLLRPPATRGITCPPPMSVEHADIW
VKSYSLYSRERYICNSGFKRKAGTSSLTECVLNKATNVAHWTTPS
LKCIRDPALVHQRPAPPSTVTTAGVTPQPESLSPSGKEPAASSPS
SNNTAATTAAIVPGSQLMPSKSPSTGTTEISSHESSHGTPSQTTA
KNWELTASASHQPPGVYPQGHSDTTVAISTSTVLLCGLSAVSLLA
CYLKSRQTPPLASVEMEAMEALPVTWGTSSRDEDLENCSHHL

In certain embodiments, the cytokine receptor is an IL-1 receptor-like 1 (ST2) polypeptide. In certain embodiments, the ST2 polypeptide is a human ST2 polypeptide. In certain embodiments, the ST2 polypeptide comprises or consists of an amino acid sequence that is at least about 80%, about 85%, about 90%, about 95%, about 96%, about 97%, about 98%, about 99%, or about 100% identical to the amino acid sequence having a UniProt Reference No: Q01638 (SEQ ID NO: 125) or a functional fragment thereof. In certain embodiments, the ST2 polypeptide can optionally comprise up to one or up to two or up to three conservative amino acid substitutions. In certain embodiments, the ST2 polypeptide comprises or consists of an amino acid sequence that is a consecutive portion of SEQ ID NO: 125, which is at least about 20, or at least about 30, or at least about 40, at least about 50, at least about 100, at least about 150, and up to about 556 amino acids in length. In certain embodiments, the ST2 polypeptide comprises or consists of the amino acid sequence of amino acids 1 to 556 or 18 to 556 of SEQ ID NO: 125. In certain embodiments, the ST2 polypeptide comprises or consists of the amino acid sequence of amino acids 1 to 556 of SEQ ID NO: 125. SEQ ID NO: 125 is provided below.

[SEQ ID NO: 125]
MGFWILAILTILMYSTAAKFSKQSWGLENEALIVRCPRQGKPSYT
VDWYYSQTNKSIPTQERNRVFASGQLLKFLPAAVADSGIYTCIVR
SPTENRTGYANVTIYKKQSDCNVPDYLMYSTVSGSEKNSKIYCPT
IDLYNWTAPLEWFKNCQALQGSRYRAHKSFLVIDNVMTEDAGDYT
CKFIHNENGANYSVTATRSFTVKDEQGFSLFPVIGAPAQNEIKEV
EIGKNANLTCSACFGKGTQFLAAVLWQLNGTKITDEGEPRIQQEE
GQNQSFSNGLACLDMVLRIADVKEEDLLLQYDCLALNLHGLRRHT
VRLSRKNPIDHHSIYCIIAVCSVFLMLINVLVIILKMFWIEATLL
WRDIAKPYKTRNDGKLYDAYVVYPRNYKSSTDGASRVEHFVHQIL
PDVLENKCGYTLCIYGRDMLPGEDVVTAVETNIRKSRRHIFILTP
QITHNKEFAYEQEVALHCALIQNDAKVILIEMEALSELDMLQAEA
LQDSLQHLMKVQGTIKWREDHIANKRSLNSKFWKHVRYQMPVPSK
IPRKASSLTPLAAQKQ

In certain embodiments, the cytokine receptor is an IL-18 receptor (IL-18R) polypeptide. In certain embodiments, the IL-18R polypeptide is a human IL-18R polypeptide. In certain embodiments, the IL-18R polypeptide comprises or consists of an amino acid sequence that is at least about 80%, about 85%, about 90%, about 95%, about 96%, about 97%, about 98%, about 99%, or about 100% identical to the amino acid sequence having a UniProt Reference No: Q13478 (SEQ ID NO: 126) or a functional fragment thereof. In certain embodiments, the IL-18R polypeptide can optionally comprise up to one or up to two or up to three conservative amino acid substitutions. In certain embodiments, the IL-18R polypeptide comprises or consists of an amino acid sequence that is a consecutive portion of SEQ ID NO: 126, which is at least about 20, or at least about 30, or at least about 40, at least about 50, at least about 100, at least about 150, and up to about 541 amino acids in length. In certain embodiments, the IL-18R polypeptide comprises or consists of the amino acid sequence of amino acids 1 to 541 or 22 to 541 of SEQ ID NO: 126. In certain embodiments, the IL-18R polypeptide comprises or consists of the amino acid sequence of amino acids 1 to 541 of SEQ ID NO: 126. SEQ ID NO: 126 is provided below.

[SEQ ID NO: 126]
MNCRELPLTLWVLISVSTAESCTSRPHITVVEGEPFYLKHCSCSL
AHEIETTTKSWYKSSGSQEHVELNPRSSSRIALHDCVLEFWPVEL
NDTGSYFFQMKNYTQKWKLNVIRRNKHSCFTERQVTSKIVEVKKF
FQITCENSYYQTLVNSTSLYKNCKKLLLENNKNPTIKKNAEFEDQ
GYYSCVHFLHHNGKLFNITKTFNITIVEDRSNIVPVLLGPKLNHV
AVELGKNVRLNCSALLNEEDVIYWMFGEENGSDPNIHEEKEMRIM
TPEGKWHASKVLRIENIGESNLNVLYNCTVASTGGTDTKSFILVR
KADMADIPGHVFTRGMIIAVLILVAVVCLVTVCVIYRVDLVLFYR
HLTRRDETLTDGKTYDAFVSYLKECRPENGEEHTFAVEILPRVLE
KHFGYKLCIFERDVVPGGAVVDEIHSLIEKSRRLIIVLSKSYMSN
EVRYELESGLHEALVERKIKIILIEFTPVTDFTFLPQSLKLLKSH
RVLKWKADKSLSYNSRFWKNLLYLMPAKTVKPGRDEPEVLPVLSE
S

In certain embodiments, the cytokine receptor is an IL-12 receptor (IL-12R) polypeptide. In certain embodiments, the IL-12R polypeptide is a human IL-12R polypeptide. In certain embodiments, the IL-12R polypeptide comprises or consists of an amino acid sequence that is at least about 80%, about 85%, about 90%, about 95%, about 96%, about 97%, about 98%, about 99%, or about 100% identical to the amino acid sequence having a UniProt Reference No: P42701 (SEQ ID NO: 127) or a functional fragment thereof. In certain embodiments, the IL-12R polypeptide can optionally comprise up to one or up to two or up to three conservative amino acid substitutions. In certain embodiments, the IL-12R polypeptide comprises or consists of an amino acid sequence that is a consecutive portion of SEQ ID NO: 127, which is at least about 20, or at least about 30, or at least about 40, at least about 50, at least about 100, at least about 150, and up to about 662 amino acids in length. In certain embodiments, the IL-12R polypeptide comprises or consists of the amino acid sequence of amino acids 1 to 662 or 24 to 662 of SEQ ID NO: 127. In certain embodiments, the IL-12R polypeptide comprises or consists of the amino acid sequence of amino acids 1 to 662 of SEQ ID NO: 127. SEQ ID NO: 127 is provided below.

[SEQ ID NO: 127]
MEPLVTWVVPLLELFLLSRQGAACRTSECCFQDPPYPDADSGSAS
GPRDLRCYRISSDRYECSWQYEGPTAGVSHFLRCCLSSGRCCYFA
AGSATRLQFSDQAGVSVLYTVTLWVESWARNQTEKSPEVTLQLYN
SVKYEPPLGDIKVSKLAGQLRMEWETPDNQVGAEVQFRHRTPSSP
WKLGDCGPQDDDTESCLCPLEMNVAQEFQLRRRQLGSQGSSWSKW
SSPVCVPPENPPQPQVRESVEQLGQDGRRRLTLKEQPTQLELPEG
CQGLAPGTEVTYRLQLHMLSCPCKAKATRTLHLGKMPYLSGAAYN
VAVISSNQFGPGLNQTWHIPADTHTEPVALNISVGTNGTTMYWPA
RAQSMTYCIEWQPVGQDGGLATCSLTAPQDPDPAGMATYSWSRES
GAMGQEKCYYITIFASAHPEKLTLWSTVLSTYHFGGNASAAGTPH
HVSVKNHSLDSVSVDWAPSLLSTCPGVLKEYVVRCRDEDSKQVSE
HPVQPTETQVTLSGLRAGVAYTVQVRADTAWLRGVWSQPQRFSIE
VQVSDWLIFFASLGSFLSILLVGVLGYLGLNRAARHLCPPLPTPC
ASSAIEFPGGKETWQWINPVDFQEEASLQEALVVEMSWDKGERTE
PLEKTELPEGAPELALDTELSLEDGDRCKAKM

In certain embodiments, the cytokine receptor is an IL-2 receptor (IL-2R) polypeptide. In certain embodiments, the IL-2R polypeptide is a human IL-2R polypeptide. In certain embodiments, the IL-2R polypeptide comprises or consists of an amino acid sequence that is at least about 80%, about 85%, about 90%, about 95%, about 96%, about 97%, about 98%, about 99%, or about 100% identical to the amino acid sequence having a UniProt Reference No: P01589 (SEQ ID NO: 128) or a functional fragment thereof. In certain embodiments, the IL-2R polypeptide can optionally comprise up to one or up to two or up to three conservative amino acid substitutions. In certain embodiments, the IL-2R polypeptide comprises or consists of an amino acid sequence that is a consecutive portion of SEQ ID NO: 128, which is at least about 20, or at least about 30, or at least about 40, at least about 50, at least about 100, at least about 150, and up to about 272 amino acids in length. In certain embodiments, the IL-2R polypeptide comprises or consists of the amino acid sequence of amino acids 1 to 272 or 22 to 272 of SEQ ID NO: 128. In certain embodiments, the IL-2R polypeptide comprises or consists of the amino acid sequence of amino acids 1 to 272 of SEQ ID NO: 128. SEQ ID NO: 128 is provided below.

[SEQ ID NO: 128]
MDSYLLMWGLLTFIMVPGCQAELCDDDPPEIPHATFKAMAYKEGT
MLNCECKRGFRRIKSGSLYMLCTGNSSHSSWDNQCQCTSSATRNT
TKQVTPQPEEQKERKTTEMQSPMQPVDQASLPGHCREPPPWENEA
TERIYHFVVGQMVYYQCVQGYRALHRGPAESVCKMTHGKTRWTQP
QLICTGEMETSQFPGEEKPQASPEGRPESETSCLVTTTDFQIQTE
MAATMETSIFTTEYQVAVAGCVELLISVLLLSGLTWQRRQRKSRR
TI

In certain embodiments, the cytokine receptor is an IL-2 receptor (IL-2R) polypeptide. In certain embodiments, the IL-2R polypeptide is a human IL-2R polypeptide. In certain embodiments, the IL-2R polypeptide comprises or consists of an amino acid sequence that is at least about 80%, about 85%, about 90%, about 95%, about 96%, about 97%, about 98%, about 99%, or about 100% identical to the amino acid sequence having a UniProt Reference No: P14784 (SEQ ID NO: 129) or a functional fragment thereof. In certain embodiments, the IL-2R polypeptide can optionally comprise up to one or up to two or up to three conservative amino acid substitutions. In certain embodiments, the IL-2R polypeptide comprises or consists of an amino acid sequence that is a consecutive portion of SEQ ID NO: 129, which is at least about 20, or at least about 30, or at least about 40, at least about 50, at least about 100, at least about 150, and up to about 551 amino acids in length. In certain embodiments, the IL-2R polypeptide comprises or consists of the amino acid sequence of amino acids 1 to 551 or 27 to 551 of SEQ ID NO: 129. In certain embodiments, the IL-2R polypeptide comprises or consists of the amino acid sequence of amino acids 1 to 551 of SEQ ID NO: 129. SEQ ID NO: 129 is provided below.

[SEQ ID NO: 129]
MAAPALSWRLPLLILLLPLATSWASAAVNGTSQFTCFYNSRANIS
CVWSQDGALQDTSCQVHAWPDRRRWNQTCELLPVSQASWACNLIL
GAPDSQKLTTVDIVTLRVLCREGVRWRVMAIQDEKPFENLRLMAP
ISLQVVHVETHRCNISWEISQASHYFERHLEFEARTLSPGHTWEE
APLLTLKQKQEWICLETLTPDTQYEFQVRVKPLQGEFTTWSPWSQ
PLAFRTKPAALGKDTIPWLGHLLVGLSGAFGFIILVYLLINCRNT
GPWLKKVLKCNTPDPSKFFSQLSSEHGGDVQKWLSSPFPSSSESP
GGLAPEISPLEVLERDKVTQLLLQQDKVPEPASLSSNHSLTSCFT
NQGYFFFHLPDALEIEACQVYFTYDPYSEEDPDEGVAGAPTGSSP
QPLQPLSGEDDAYCTFPSRDDLLLFSPSLLGGPSPPSTAPGGSGA
GEERMPPSLQERVPRDWDPQPLGPPTPGVPDLVDFQPPPELVLRE
AGEEVPDAGPREGVSFPWSRPPGQGEFRALNARLPLNTDAYLSLQ
ELQGQDPTHLV

In certain embodiments, the cytokine receptor is an IL-4 receptor (IL-4R) polypeptide. In certain embodiments, the IL-4R polypeptide is a human IL-4R polypeptide. In certain embodiments, the IL-4R polypeptide comprises or consists of an amino acid sequence that is at least about 80%, about 85%, about 90%, about 95%, about 96%, about 97%, about 98%, about 99%, or about 100% identical to the amino acid sequence having a UniProt Reference No: P24394 (SEQ ID NO: 134) or a functional fragment thereof. In certain embodiments, the IL-4R polypeptide can optionally comprise up to one or up to two or up to three conservative amino acid substitutions. In certain embodiments, the IL-4R polypeptide comprises or consists of an amino acid sequence that is a consecutive portion of SEQ ID NO: 134, which is at least about 20, or at least about 30, or at least about 40, at least about 50, at least about 100, at least about 150, and up to about 825 amino acids in length. In certain embodiments, the IL-4R polypeptide comprises or consists of the amino acid sequence of amino acids 1 to 825, 26 to 825, or 233 to 285 of SEQ ID NO: 134. In certain embodiments, the IL-15R polypeptide comprises or consists of the amino acid sequence of amino acids 1 to 825 of SEQ ID NO: 134. SEQ ID NO: 134 is provided below.

[SEQ ID NO: 134]
MGWLCSGLLFPVSCLVLLQVASSGNMKVLQEPTCVSDYMSISTCE
WKMNGPTNCSTELRLLYQLVFLLSEAHTCIPENNGGAGCVCHLLM
DDVVSADNYTLDLWAGQQLLWKGSFKPSEHVKPRAPGNLTVHTNV
SDTLLLTWSNPYPPDNYLYNHLTYAVNIWSENDPADFRIYNVTYL
EPSLRIAASTLKSGISYRARVRAWAQCYNTTWSEWSPSTKWHNSY
REPFEQHLLLGVSVSCIVILAVCLLCYVSITKIKKEWWDQIPNPA
RSRLVAIIIQDAQGSQWEKRSRGQEPAKCPHWKNCLTKLLPCFLE
HNMKRDEDPHKAAKEMPFQGSGKSAWCPVEISKTVLWPESISVVR
CVELFEAPVECEEEEEVEEEKGSFCASPESSRDDFQEGREGIVAR
LTESLFLDLLGEENGGFCQQDMGESCLLPPSGSTSAHMPWDEFPS
AGPKEAPPWGKEQPLHLEPSPPASPTQSPDNLTCTETPLVIAGNP
AYRSFSNSLSQSPCPRELGPDPLLARHLEEVEPEMPCVPQLSEPT
TVPQPEPETWEQILRRNVLQHGAAAAPVSAPTSGYQEFVHAVEQG
GTQASAVVGLGPPGEAGYKAFSSLLASSAVSPEKCGFGASSGEEG
YKPFQDLIPGCPGDPAPVPVPLFTFGLDREPPRSPQSSHLPSSSP
EHLGLEPGEKVEDMPKPPLPQEQATDPLVDSLGSGIVYSALTCHL
CGHLKQCHGQEDGGQTPVMASPCCGCCCGDRSSPPTTPLRAPDPS
PGGVPLEASLCPASLAPSGISEKSKSSSSFHPAPGNAQSSSQTPK
IVNFVSVGPTYMRVS

In certain embodiments, the cytokine receptor is an IL-7 receptor (IL-7R) polypeptide. In certain embodiments, the IL-7R polypeptide is a human IL-7R polypeptide. In certain embodiments, the IL-7R polypeptide comprises or consists of an amino acid sequence that is at least about 80%, about 85%, about 90%, about 95%, about 96%, about 97%, about 98%, about 99%, or about 100% identical to the amino acid sequence having a UniProt Reference No: P16871 (SEQ ID NO: 135) or a functional fragment thereof. In certain embodiments, the IL-7R polypeptide can optionally comprise up to one or up to two or up to three conservative amino acid substitutions. In certain embodiments, the IL-7R polypeptide comprises or consists of an amino acid sequence that is a consecutive portion of SEQ ID NO: 135, which is at least about 20, or at least about 30, or at least about 40, at least about 50, at least about 100, at least about 150, and up to about 459 amino acids in length. In certain embodiments, the IL-7R polypeptide comprises or consists of the amino acid sequence of amino acids 1 to 459 or 21 to 459 of SEQ ID NO: 135. In certain embodiments, the IL-7R polypeptide comprises or consists of the amino acid sequence of amino acids 1 to 459 of SEQ ID NO: 135. SEQ ID NO: 135 is provided below.

[SEQ ID NO: 135]
MTILGTTFGMVFSLLQVVSGESGYAQNGDLEDAELDDYSFSCYSQ
LEVNGSQHSLTCAFEDPDVNITNLEFEICGALVEVKCLNFRKLQE
IYFIETKKELLIGKSNICVKVGEKSLICKKIDLTTIVKPEAPFDL
SVVYREGANDFVVTFNTSHLQKKYVKVLMHDVAYRQEKDENKWTH
VNLSSTKLTLLQRKLQPAAMYEIKVRSIPDHYFKGFWSEWSPSYY
FRTPEINNSSGEMDPILLTISILSFFSVALLVILACVLWKKRIKP
IVWPSLPDHKKTLEHLCKKPRKNLNVSFNPESFLDCQIHRVDDIQ
ARDEVEGFLQDTFPQQLEESEKQRLGGDVQSPNCPSEDVVITPES
FGRDSSLTCLAGNVSACDAPILSSSRSLDCRESGKNGPHVYQDLL
LSLGTTNSTLPPPFSLQSGILTLNPVAQGQPILTSLGSNQEEAYV
TMSSFYQNQ

In certain embodiments, the cytokine receptor is an IL-9 receptor (IL-9R) polypeptide. In certain embodiments, the IL-9R polypeptide is a human IL-9R polypeptide. In certain embodiments, the IL-9R polypeptide comprises or consists of an amino acid sequence that is at least about 80%, about 85%, about 90%, about 95%, about 96%, about 97%, about 98%, about 99%, or about 100% identical to the amino acid sequence having a UniProt Reference No: Q01113 (SEQ ID NO: 136) or a functional fragment thereof. In certain embodiments, the IL-9R polypeptide can optionally comprise up to one or up to two or up to three conservative amino acid substitutions. In certain embodiments, the IL-9R polypeptide comprises or consists of an amino acid sequence that is a consecutive portion of SEQ ID NO: 136, which is at least about 20, or at least about 30, or at least about 40, at least about 50, at least about 100, at least about 150, and up to about 521 amino acids in length. In certain embodiments, the IL-9R polypeptide comprises or consists of the amino acid sequence of amino acids 1 to 521 or 41 to 521 of SEQ ID NO: 136. In certain embodiments, the IL-9R polypeptide comprises or consists of the amino acid sequence of amino acids 1 to 521 of SEQ ID NO: 136. SEQ ID NO: 136 is provided below.

[SEQ ID NO: 136]
MGLGRCIWEGWTLESEALRRDMGTWLLACICICTCVCLGVSVTGEGQGPRSRTFTCLINNILRI
DCHWSAPELGQGSSPWLLFTSNQAPGGTHKCILRGSECTVVLPPEAVLVPSDNFTITFHHCMSG
REQVSLVDPEYLPRRHVKLDPPSDLQSNISSGHCILTWSISPALEPMTTLLSYELAFKKQEEAW
EQAQHRDHIVGVTWLILEAFELDPGFIHEARLRVQMATLEDDVVEEERYTGQWSEWSQPVCFQA
PQRQGPLIPPWGWPGNTLVAVSIFLLLTGPTYLLFKLSPRVKRIFYQNVPSPAMFFQPLYSVHN
GNFQTWMGAHGAGVLLSQDCAGTPQGALEPCVQEATALLTCGPARPWKSVALEEEQEGPGTRLP
GNLSSEDVLPAGCTEWRVQTLAYLPQEDWAPTSLTRPAPPDSEGSRSSSSSSSSNNNNYCALGC
YGGWHLSALPGNTQSSGPIPALACGLSCDHQGLETQQGVAWVLAGHCQRPGLHEDLQGMLLPSV
LSKARSWTF

In certain embodiments, the cytokine receptor is an IL-21 receptor (IL-21R) polypeptide. In certain embodiments, the IL-21R polypeptide is a human IL-21R polypeptide. In certain embodiments, the IL-21R polypeptide comprises or consists of an amino acid sequence that is at least about 80%, about 85%, about 90%, about 95%, about 96%, about 97%, about 98%, about 99%, or about 100% identical to the amino acid sequence having a UniProt Reference No: Q9HBE5 (SEQ ID NO: 137) or a functional fragment thereof. In certain embodiments, the IL-21R polypeptide can optionally comprise up to one or up to two or up to three conservative amino acid substitutions. In certain embodiments, the IL-21R polypeptide comprises or consists of an amino acid sequence that is a consecutive portion of SEQ ID NO: 137, which is at least about 20, or at least about 30, or at least about 40, at least about 50, at least about 100, at least about 150, and up to about 538 amino acids in length. In certain embodiments, the IL-21R polypeptide comprises or consists of the amino acid sequence of amino acids 1 to 538 or 20 to 538 of SEQ ID NO: 137. In certain embodiments, the IL-21R polypeptide comprises or consists of the amino acid sequence of amino acids 1 to 538 of SEQ ID NO: 137. SEQ ID NO: 137 is provided below.

[SEQ ID NO: 137]
MPRGWAAPLLLLLLQGGWGCPDLVCYTDYLQTVICILEMWNLHPSTLTLTWQDQYEELKDEATS
CSLHRSAHNATHATYTCHMDVFHFMADDIFSVNITDQSGNYSQECGSFLLAESIKPAPPFNVTV
TFSGQYNISWRSDYEDPAFYMLKGKLQYELQYRNRGDPWAVSPRRKLISVDSRSVSLLPLEFRK
DSSYELQVRAGPMPGSSYQGTWSEWSDPVIFQTQSEELKEGWNPHLLLLLLLVIVFIPAFWSLK
THPLWRLWKKIWAVPSPERFFMPLYKGCSGDFKKWVGAPFTGSSLELGPWSPEVPSTLEVYSCH
PPRSPAKRLQLTELQEPAELVESDGVPKPSFWPTAQNSGGSAYSEERDRPYGLVSIDTVTVLDA
EGPCTWPCSCEDDGYPALDLDAGLEPSPGLEDPLLDAGTTVLSCGCVSAGSPGLGGPLGSLLDR
LKPPLADGEDWAGGLPWGGRSPGGVSESEAGSPLAGLDMDTFDSGFVGSDCSSPVECDFTSPGD
EGPPRSYLRQWVVIPPPLSSPGPQAS

Additional information on cytokine receptors encompassed by the presently disclosed subject matter can be found in Zhou et al., Frontiers in Immunology 14 (2023): 1090311, which is incorporated by reference in its entirety.

### 2.4. Antigen Recognizing Receptors

In certain embodiments, the cells disclosed herein further comprise an antigen-recognizing receptor. In certain embodiments, the antigen-recognizing receptor targets a second antigen. The second antigen can be a tumor antigen or a pathogen antigen such as those disclosed in Section 2.1.1. In certain embodiments the chimeric receptor is a chimeric antigen receptor (CAR).

### 2.4.1. Chimeric Antigen Receptors (CARs)

In certain embodiments, the antigen-recognizing receptor is a chimeric antigen receptor (CAR). CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARS. “First generation” CARs are typically composed of an extracellular antigen-binding domain (e.g., an scFv) that binds to a target antigen, and an intracellular signaling domain. In certain embodiments, the CAR further comprises a transmembrane domain. “First generation” CARs can provide de novo antigen recognition and cause activation of both CD4+ and CD8+ T cells through their CD35 chain signaling domain in a single fusion molecule, independent of HLA-mediated antigen presentation. “Second generation” CARs include a signaling domain of a co-stimulatory molecule (e.g., CD28, 4-1BB, ICOS, OX40, CD27, CD40, NKG2D, DAP-10, CD2, CD150, CD226) to the intracellular signaling domain of the CAR to provide co-stimulation signals to the cell (e.g., T cell or NK cell). “Second generation” CARs comprise those that provide both co-stimulation (e.g., CD28 or 4-1BB) and activation (CD32). “Third generation” CARs comprise those that provide multiple co-stimulation (e.g., CD28 and 4-1BB) and activation (CD35).

In certain embodiments, the antigen-recognizing receptor is a CAR comprising an extracellular antigen-binding domain that binds to an antigen (e.g., one disclosed in Section 2.1.2) and an intracellular signaling domain. In certain embodiments, the CAR further comprises a transmembrane domain. In certain embodiments, the CAR further comprises a hinge/spacer region.

### 2.4.1.1. Transmembrane Domain and Hinge/Spacer Region (CARs)

In certain embodiments, the transmembrane domain of the CAR comprises a hydrophobic alpha helix that spans at least a portion of the membrane. Different transmembrane domains result in different receptor stability. After antigen recognition, receptors cluster and a signal are transmitted to the cell. In accordance with the presently disclosed subject matter, the transmembrane domain of the CAR can comprise a native or modified transmembrane domain of CD8 or a fragment thereof, a native or modified transmembrane domain of CD28 or a fragment thereof, a native or modified transmembrane domain of CD35 or a fragment thereof, a native or modified transmembrane domain of CD4 or a fragment thereof, a native or modified transmembrane domain of 4-1BB or a fragment thereof, a native or modified transmembrane domain of OX40 or a fragment thereof, a native or modified transmembrane domain of ICOS or a fragment thereof, a native or modified transmembrane domain of CD84 or a fragment thereof, a native or modified transmembrane domain of CD166 or a fragment thereof, a native or modified transmembrane domain of CD8a or a fragment thereof, a native or modified transmembrane domain of CD8b or a fragment thereof, a native or modified transmembrane domain of ICAM-1 or a fragment thereof, a native or modified transmembrane domain of CTLA-4 or a fragment thereof, a native or modified transmembrane domain of CD27 or a fragment thereof, a native or modified transmembrane domain of CD40 or a fragment thereof, NKGD2 or a fragment thereof, or a combination thereof.

In certain embodiments, the transmembrane domain of the CAR comprises a CD8 polypeptide (e.g., a transmembrane domain of CD8 or a fragment thereof). In certain embodiments, the transmembrane domain of the CAR comprises a transmembrane domain of human CD8 or a fragment thereof. In certain embodiments, the CD8 polypeptide comprises or consists of an amino acid sequence that is at least about 85%, about 90%, about 95%, about 96%, about 97%, about 98%, about 99% or about 100% homologous or identical to the amino acid sequence having a NCBI Reference No: NP_001139345.1 (SEQ ID NO: 38) or a fragments thereof, and/or may optionally comprise up to one or up to two or up to three conservative amino acid substitutions. In certain embodiments, the CD8 polypeptide comprises or consists of an amino acid sequence that is a consecutive portion of SEQ ID NO: 38, which is at least 20, or at least 30, or at least 40, or at least 50, and up to 235 amino acids in length. In certain embodiments, the CD8 polypeptide comprises or consists of an amino acid sequence of amino acids 1 to 235, 1 to 50, 50 to 100, 100 to 150, 150 to 200, 137 to 209 or 200 to 235 of SEQ ID NO: 38. In certain embodiments, the transmembrane domain of the CAR comprises a CD8 polypeptide comprising or consisting of amino acids 137 to 209 of SEQ ID NO: 38. SEQ ID NO: 38 is provided below.

[SEQ ID NO: 38]
MALPVTALLLPLALLLHAARPSQFRVSPLDRTWNLGETVELKCQVLLSNPTSGCSWLFQPRGAA
ASPTFLLYLSQNKPKAAEGLDTQRFSGKRLGDTFVLTLSDFRRENEGYYFCSALSNSIMYFSHF
VPVFLPAKPTTTPAPRPPTPAPTIASQPLSLRPEACRPAAGGAVHTRGLDFACDIYIWAPLAGT
CGVLLLSLVITLYCNHRNRRRVCKCPRPVVKSGDKPSLSARYV

In certain embodiments, the transmembrane domain of the CAR comprises a transmembrane domain of mouse CD8 or a fragment thereof. In certain embodiments, the CD8 polypeptide comprises or consists of an amino acid sequence that is at least about 85%, about 90%, about 95%, about 96%, about 97%, about 98%, about 99% or about 100% homologous or identical to the amino acid sequence having a NCBI Reference No: AAA92533.1 (SEQ ID NO: 39) or a fragment thereof, and/or may optionally comprise up to one or up to two or up to three conservative amino acid substitutions. In certain embodiments, the CD8 polypeptide comprises or consists of an amino acid sequence that is a consecutive portion of SEQ ID NO: 39, which is at least about 20, or at least about 30, or at least about 40, or at least about 50, or at least about 60, or at least about 70, or at least about 100, or at least about 200, and up to 247 amino acids in length. In certain embodiments, the CD8 polypeptide comprises or consists of an amino acid sequence of amino acids 1 to 247, 1 to 50, 50 to 100, 100 to 150, 150 to 200, 151 to 219, or 200 to 247 of SEQ ID NO: 39. In certain embodiments, the transmembrane domain of the CAR comprises a CD8 polypeptide comprising or consisting of amino acids 151 to 219 of SEQ ID NO: 39. SEQ ID NO: 39 is provided below.

[SEQ ID NO: 39]
MASPLTRFLSLNLLLMGESIILGSGEAKPQAPELRIFPKKMDAELGQKVDLVCEVLGSVSQGCS
WLFQNSSSKLPQPTFVVYMASSHNKITWDEKLNSSKLFSAVRDTNNKYVLTLNKFSKENEGYYF
CSVISNSVMYFSSVVPVLQKVNSTTTKPVLRTPSPVHPTGTSQPQRPEDCRPRGSVKGTGLDFA
CDIYIWAPLAGICVAPLLSLIITLICYHRSRKRVCKCPRPLVRQEGKPRPSEKIV

In certain embodiments, the transmembrane domain of a presently disclosed CAR comprises a CD28 polypeptide (e.g., a transmembrane domain of CD28 or a fragment thereof).

In certain embodiments, the transmembrane domain of the CAR comprises a transmembrane domain of human CD28 or a fragment thereof. In certain embodiments, the CD28 polypeptide comprises or consists of an amino acid sequence that is at least about 85%, about 90%, about 95%, about 96%, about 97%, about 98%, about 99% or 100% homologous or identical to the amino acid sequence having a NCBI Reference No: NP_006130 (SEQ ID NO: 40) or a fragment thereof, and/or may optionally comprise up to one or up to two or up to three conservative amino acid substitutions. In non-limiting certain embodiments, the CD28 polypeptide comprises or consists of an amino acid sequence that is a consecutive portion of SEQ ID NO: 40 which is at least 20, or at least 30, or at least 40, or at least 50, and up to 220 amino acids in length. In certain embodiments, the CD28 polypeptide comprises or consists of an amino acid sequence of amino acids 1 to 220, 1 to 50, 50 to 100, 100 to 150, 150 to 200, 153 to 179, or 200 to 220 of SEQ ID NO: 40. In certain embodiments, the transmembrane domain of the CAR comprises a CD28 polypeptide comprising or consisting of amino acids 153 to 179 of SEQ ID NO: 40.

In certain embodiments, the transmembrane domain of the CAR comprises a CD28 polypeptide (e.g., a transmembrane domain of mouse CD28 or a fragment thereof). In certain embodiments, the CD28 polypeptide comprises or consists of an amino acid sequence that is at least about 85%, about 90%, about 95%, about 96%, about 97%, about 98%, about 99% or 100% homologous or identical to the amino acid sequence having a NCBI Reference No: NP_031668.3 (SEQ ID No: 42) or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 42, which is at least 20, or at least 30, or at least 40, or at least 50, and up to 218 amino acids in length. In certain embodiments, the CD28 polypeptide comprises or consists of an amino acid sequence of amino acids 1 to 220, 1 to 50, 50 to 100, 100 to 150, 150 to 200, 151 to 177, or 200 to 218 of SEQ ID NO: 42. In certain embodiments, the transmembrane domain of the CAR comprises a CD28 polypeptide comprising or consisting of amino acids 151 to 177 of SEQ ID NO: 42.

In certain non-limiting embodiments, the CAR further comprises a spacer region that links the extracellular antigen-binding domain to the transmembrane domain. The spacer region can be flexible enough to allow the antigen binding domain to orient in different directions to facilitate antigen recognition while preserving the activating activity of the CAR.

In certain embodiments, the hinge/spacer region of the CAR comprises a native or modified hinge region of CD8 or a fragment thereof, a native or modified hinge region of CD28 or a fragment thereof, a native or modified hinge region of CD32 or a fragment thereof, a native or modified hinge region of CD40 or a fragment thereof, a native or modified hinge region of 4-1BB or a fragment thereof, a native or modified hinge region of OX40 or a fragment thereof, a native or modified hinge region of CD84 or a fragment thereof, a native or modified hinge region of CD166 or a fragment thereof, a native or modified hinge region of CD8a or a fragment thereof, a native or modified hinge region of CD8b or a fragment thereof, a native or modified hinge region of ICOS or a fragment thereof, a native or modified hinge region of ICAM-1 or a fragment thereof, a native or modified hinge region of CTLA-4 or a fragment thereof, a native or modified hinge region of CD27 or a fragment thereof, a native or modified hinge region of CD40 or a fragment thereof, a native or modified hinge region of NKGD2 or a fragment thereof, a synthetic polypeptide (not based on a protein associated with the immune response), or a combination thereof. The hinge/spacer region can be the hinge region from IgG1, or the CH₂CH₃ region of immunoglobulin and portions of CD3, a portion of a CD8 polypeptide (e.g., a portion of SEQ ID NO: 38 or 39), a portion of a CD28 polypeptide (e.g., a portion of SEQ ID NO: 40 or 42), a variation of any of the foregoing which is at least about 80%, at least about 85%, at least about 90%, at least about 95%, or at least about 100% homologous or identical thereto, or a synthetic spacer sequence.

In certain embodiments, the hinge/spacer region of the CAR comprises a CD28 polypeptide comprising or consisting of amino acids 114 to 152 of SEQ ID NO: 40. In certain embodiments, the hinge/spacer region of the CAR comprises or consists of an amino acid sequence that is at least about 80%, at least about 85%, at least about 90%, at least about 95%, or at least about 99% identical to the amino acid sequence set forth in SEQ ID NO: 20. In certain embodiments, the hinge/spacer region of the CAR comprises or consists of the amino acid sequence set forth in SEQ ID NO: 20.

### 2.4.1.2. Intracellular Signaling Domain (CARs)

In certain embodiments, the antigen recognizing receptor comprises an intracellular signaling domain. In certain non-limiting embodiments, the intracellular signaling domain of the CAR comprises a CD35 polypeptide. CD3ζ can activate or stimulate a cell (e.g., a cell of the lymphoid lineage, e.g., a T cell). Wild type (“native”) CD3λ comprises three functional immunoreceptor tyrosine-based activation motifs (ITAMs), three functional basic-rich stretch (BRS) regions (BRS1, BRS2 and BRS3). CD3ζ transmits an activation signal to the cell (e.g., a cell of the lymphoid lineage, e.g., a T cell) after antigen is bound. The intracellular signaling domain of the CD3-chain is the primary transmitter of signals from endogenous TCRs.

In certain embodiments, the intracellular signaling domain of the CAR comprises a native CD3ζ. In certain embodiments, the CD3 polypeptide comprises or consists of an amino acid sequence that is at least about 85%, about 90%, about 95%, about 96%, about 97%, about 98%, about 99% or about 100% homologous or identical to the amino acid sequence having a NCBI Reference No: NP_932170 (SEQ ID NO: 43) or a fragment thereof, and/or may optionally comprise up to one or up to two or up to three conservative amino acid substitutions. In certain non-limiting embodiments, the CD32 polypeptide comprises or consists of an amino acid sequence that is a consecutive portion of SEQ ID NO: 43, which is at least 20, or at least 30, or at least 40, or at least 50, and up to 164 amino acids in length. In certain embodiments, the CD32 polypeptide comprises or consists of an amino acid sequence of amino acids 1 to 164, 1 to 50, 50 to 100, 52 to 164, 100 to 150, or 150 to 164 of SEQ ID NO: 43. In certain embodiments, the intracellular signaling domain of the CAR comprises a CD32 polypeptide comprising or consisting of amino acids 52 to 164 of SEQ ID NO: 43. SEQ ID NO: 43 is provided below:

[SEQ ID NO: 43]
MKWKALFTAAILQAQLPITEAQSFGLLDPKLCYLLDGILFIYGVILTAL
FLRVKFSRSADAPAYQQGQNQLYNELNLGRREEYDVLDKRRGRDPEMGG
KPQRRKNPQEGLYNELQKDKMAEAYSEIGMKGERRRGKGHDGLYQGLST
ATKDTYDALHMQALPPR

In certain embodiments, the intracellular signaling domain of the CAR comprises a CD3 polypeptide comprising or consisting of the amino acid sequence set forth in SEQ ID NO: 44. SEQ ID NO: 44 is provided below.

[SEQ ID NO: 44]
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

An exemplary nucleotide sequence encoding the amino acid sequence of SEQ ID NO: 44 is set forth in SEQ ID NO: 45, which is as provided below.

[SEQ ID NO: 45]
AGAGTGAAGTTCAGCAGGAGCGCAGACGCCCCCGCGTACCAGCAGGGCCAGAACCAGCTCTATA
ACGAGCTCAATCTAGGACGAAGAGAGGAGTACGATGTTTTGGACAAGAGACGTGGCCGGGACCC
TGAGATGGGGGGAAAGCCGAGAAGGAAGAACCCTCAGGAAGGCCTGTACAATGAACTGCAGAAA
GATAAGATGGCGGAGGCCTACAGTGAGATTGGGATGAAAGGCGAGCGCCGGAGGGGCAAGGGGC
ACGATGGCCTTTACCAGGGTCTCAGTACAGCCACCAAGGACACCTACGACGCCCTTCACATGCA
GGCCCTGCCCCCTCGC

In certain embodiments, the intracellular signaling domain of the CAR further comprises at least a co-stimulatory signaling region. In certain embodiments, the co-stimulatory signaling region comprises at least one co-stimulatory molecule or a fragment thereof. In certain embodiments, the co-stimulatory signaling region comprises an intracellular domain of at least one co-stimulatory molecule or a fragment thereof.

As used herein, a “co-stimulatory molecule” refers to a cell surface molecule other than antigen receptor or its ligand that can provide an efficient response of lymphocytes to an antigen. In certain embodiments, a co-stimulatory molecule can provide optimal lymphocyte activation. Non-limiting examples of co-stimulatory molecules include CD28, 4-1BB, OX40, ICOS, DAP-10, CD27, CD40, NKGD2, CD2, FN14, HVEM, LTBR, CD28H, TNFR1, TNFR2, BAFF-R, BCMA, TACI, TROY, RANK, CD40, CD27, CD30, EDAR, XEDAR, GITR, DR6, and NGFR, and combinations thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen-recognizing receptor (e.g., a chimeric antigen receptor (CAR)) binds to its target antigen. As one example, a 4-1BB ligand (i.e., 4-1BBL) may bind to 4-1BB for providing an intracellular signal that in combination with a CAR signal induces an effector cell function of the CAR⁺ T cell.

In certain embodiments, the intracellular signaling domain of the CAR comprises a co-stimulatory signaling region that comprises a CD28 polypeptide, e.g., an intracellular domain of CD28 or a fragment thereof. In certain embodiments, the intracellular signaling domain of the CAR comprises a co-stimulatory signaling region that comprises a CD28 polypeptide, e.g., an intracellular domain of human CD28 or a fragment thereof. In certain embodiments, the CD28 polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 40 or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 40, which is at least 20, or at least 30, or at least 40, or at least 50, and up to 220 amino acids in length. In certain embodiments, the CD28 polypeptide comprises or consists of an amino acid sequence of amino acids 1 to 220, 1 to 50, 50 to 100, 100 to 150, 114 to 220, 150 to 200, 180 to 220, or 200 to 220 of SEQ ID NO: 40. In certain embodiments, the intracellular signaling domain of the CAR comprises a co-stimulatory signaling region that comprises a CD28 polypeptide comprising or consisting of an amino acid sequence of amino acids 180 to 220 of SEQ ID NO: 40.

An exemplary nucleotide sequence encoding amino acids 180 to 220 of SEQ ID NO: 40 is set forth in SEQ ID NO: 19, which is provided below.

[SEQ ID NO: 19]
AGGAGTAAGAGGAGCAGGCTCCTGCACAGTGACTACATGAACATGACTC
CCCGCCGCCCCGGGCCCACCCGCAAGCATTACCAGCCCTATGCCCCACC
ACGCGACTTCGCAGCCTATCGCTCC

In certain embodiments, the intracellular signaling domain of the CAR comprises a co-stimulatory signaling region that comprises a CD28 polypeptide, e.g., an intracellular domain of mouse CD28 or a fragment thereof. In certain embodiments, the CD28 polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 42 or a fragment thereof, and/or may optionally comprise up to one or up to two or up to three conservative amino acid substitutions. In non-limiting certain embodiments, the CD28 polypeptide comprises or consists of an amino acid sequence that is a consecutive portion of SEQ ID NO: 42, which is at least about 20, or at least about 30, or at least about 40, or at least about 50, and up to 218 amino acids in length. In certain embodiments, the CD28 polypeptide comprises or consists of an amino acid sequence of amino acids 1 to 218, 1 to 50, 50 to 100, 100 to 150, 150 to 218, 178 to 218, or 200 to 218 of SEQ ID NO: 42. In certain embodiments, the co-stimulatory signaling region of a presently disclosed CAR comprises a CD28 polypeptide that comprises or consists of the amino acids 178 to 218 of SEQ ID NO: 42.

In certain embodiments, the co-stimulatory signaling region of a presently disclosed CAR comprises a CD28 polypeptide comprising a mutated YMNM motif. CD28 is a transmembrane protein that plays a critical role in T cell activation through its function as a costimulatory molecule. CD28 possesses an intracellular domain, which comprises intracellular motifs that are critical for the effective signaling of CD28. In certain embodiments, the CD28 intracellular domain comprises intracellular subdomains (also known as “intracellular motifs”) that regulate signaling pathways post TCR-stimulation. CD28 includes three intracellular motifs: a YMNM motif, and two proline-rick motifs: PRRP motif (SEQ ID NO: 139), and PYAP motif (SEQ ID NO: 140). The CD28 intracellular motifs can serve as docking sites for a number of adaptor molecules that interact with these motifs through their SH2 or SH3 domains. Such interaction transduces downstream signals terminating on transcription factors that regulate gene expression. For example, a native YMNM motif binds to a p85 subunit of a phosphoinositide 3-kinase (PI3K). A native YMNM motif also binds to growth factor receptor-bound protein 2 (Grb2) and/or Grb2-related adaptor protein 2 (GADS). Grb2 binds to Gab1 and Gab2, which in turn can recruit the p85 subunit of a PI3K.

In certain embodiments, a native YMNM motif consists of the amino acid sequence set forth in YMNM (SEQ ID NO: 47). In certain embodiments, a native YMNM motif binds to the p85 subunit of PI3K via a consensus sequence YMxM (SEQ ID NO: 48), wherein x is not an asparagine (N). In certain embodiments, a native YMNM motif binds to Grb2 and/or GADs via a consensus sequence YxNx (SEQ ID NO: 49), wherein x is not a methionine (M).

In certain embodiments, the CD28 polypeptide comprising a presently disclosed mutated YMNM motif has reduced recruitment of the p85 subunit of a PI3K as compared to a CD28 molecule comprising a native YMNM motif. In certain embodiments, the p85 subunit of a PI3K does not bind to the mutated YMNM motif, thereby reducing the recruitment of the p85 subunit of a PI3K to the CD28 polypeptide. The mutated YMNM motif that blocks the binding of the p85 subunit of a PI3K retains its binding to Grb2 and/or GADS. Thus, downstream signaling of Grb2/GADS remains intact, e.g., downstream signaling leading to IL-2 secretion remains intact. Such mutated YMNM motif is referred to as “GADS/Grb2-permitting mutant”.

In certain embodiments, the mutated YMNM binds to the p85 subunit of a PI3K, but does not bind to Grb2 and/or GADS. Since the binding of PI3K p85 is retained, the downstream signaling of PI3K retains intact. Since the binding of Grb2/GADS is blocked, the recruitment of PI3K p85 subunit, which is triggered by the binding of Grb2 to Gab1 and Gab2, is reduced or blocked. In addition, the downstream signaling of Grb2/GADS is blocked. Such mutated YMNM motif is referred to as “PI3K-permissive mutant”.

In certain embodiments, the mutated YMNM does not bind to the p85 subunit of a PI3K, and does not bind to Grb2 and/or GADS. Such mutated YMNM motif is referred to as “non-functional mutant”. Non-functional mutants do not provide binding of PI3K, Grb2, or GADS to CD28 at the YMNM motif, but do not preclude these signaling molecules from binding elsewhere in the CD28 molecule.

In certain embodiments, the mutated YMNM retains only one methionine residue of the two methionine residues present in the YMNM motif, i.e. YMxx or YxxM. These motifs potentially modulate signaling via PI3K by limiting how many methionine residues can bind the p85 subunit of PI3K. Such mutated YMNM motif is referred to as “hybrid ‘HEMI’ mutant”.

In certain embodiments, the mutated YMNM motif is a GADS/Grb-2 permitting mutant. In certain embodiments, the mutated YMNM motif consists of the amino acid sequence set forth in YxNx (SEQ ID NO: 49), wherein x is not a methionine (M). In certain embodiments, x is selected from the group consisting of amino acids A, R, N, D, C, E, Q, G, H, I, K, F, P, S, T, W, Y, V, and L. In certain embodiments, the mutated YMNM motif consists of the amino acid sequence set forth in YENV (SEQ ID NO: 50), YSNV (SEQ ID NO: 51), YKNL (SEQ ID NO: 52), YENQ (SEQ ID NO: 53), YKNI (SEQ ID NO: 54), YINQ (SEQ ID NO: 55), YHNK (SEQ ID NO: 56), YVNQ (SEQ ID NO: 57), YLNP (SEQ ID NO: 58), YLNT (SEQ ID NO: 59), YDND (SEQ ID NO: 60), YENI (SEQ ID NO: 61), YENL (SEQ ID NO: 62), YKNQ (SEQ ID NO: 63), YKNV (SEQ ID NO: 64), or YANG (SEQ ID NO: 65). In certain embodiments, the mutated YMNM motif consists of the amino acid sequence set forth in YSNV (SEQ ID NO: 51). In certain embodiments, the mutated YMNM motif consists of the amino acid sequence set forth in YKNI (SEQ ID NO: 54). In certain embodiments, the mutated YMNM motif consists of the amino acid sequence set forth in YENV (SEQ ID NO: 50). In certain embodiments, the mutated YMNM motif consists of the amino acid sequence set forth in YKNL (SEQ ID NO: 52).

In certain embodiments, the mutated YMNM motif is a PI3K-permissive mutant. In certain embodiments, the mutated YMNM motif consists of the amino acid sequence set forth in YMxM (SEQ ID NO: 48), wherein x is not an asparagine (N). In certain embodiments, x is selected from the group consisting of amino acids A, R, D, C, E, Q, G, H, I, K, M, F, P, S, T, W, Y, V, and L. In certain embodiments, the mutated YMNM motif consists of the amino acid sequence set forth in YMDM (SEQ ID NO: 66), YMPM (SEQ ID NO: 67), YMRM (SEQ ID NO: 68), or YMSM (SEQ ID NO: 69). In certain embodiments, the mutated YMNM motif consists of the amino acid sequence set forth in YMDM (SEQ ID NO: 66).

In certain embodiments, the mutated YMNM motif consists of the amino acid sequence set forth in YbxM (SEQ ID NO: 70), wherein x is not an asparagine (N), and b is not a methionine (M). In certain embodiments, x is selected from the group consisting of amino acids A, R, D, C, E, Q, G, H, I, K, M, F, P, S, T, W, Y, V, and L. In certain embodiments, b is selected from the group consisting of amino acids A, R, N, C, E, Q, G, H, I, K, N, F, P, S, T, W, Y, V, and L. In certain embodiments, the mutated YMNM motif consists of the amino acid sequence set forth in YTHM (SEQ ID NO: 71), YVLM (SEQ ID NO: 72), YIAM (SEQ ID NO: 73), YVEM (SEQ ID NO: 74), YVKM (SEQ ID NO: 75), or YVPM (SEQ ID NO: 76).

In certain embodiments, the mutated YMNM motif consists of the amino acid sequence set forth in YMxb (SEQ ID NO: 77), wherein x is not an asparagine (N), and b is not a methionine (M). In certain embodiments, x is selected from the group consisting of amino acids A, R, D, C, E, Q, G, H, I, K, M, F, P, S, T, W, Y, V, and L. In certain embodiments, b is selected from the group consisting of amino acids A, R, N, C, E, Q, G, H, I, K, N, F, P, S, T, W, Y, V, and L. In certain embodiments, the mutated YMNM motif consists of the amino acid sequence set forth in YMAP (SEQ ID NO: 78).

Certain mutated YMNM motifs are described in Mol Cell Proteomics. 2010 November; 9 (11): 2391-404; Virology. 2015 May; 0:568-577, both of which are incorporated by reference herein in its entirety.

In certain embodiments, the mutated YMNM motif is a hybrid ‘HEMI’ mutant. In certain embodiments, the mutated YMNM motif consists of the amino acid sequence set forth in YMNx (SEQ ID NO: 79) or YxNM (SEQ ID NO: 80), wherein x is not a methionine (M). In certain embodiments, x is selected from the group consisting of amino acids A, R, N, C, E, Q, G, H, I, K, N, F, P, S, T, W, Y, V, and L. In certain embodiments, the mutated YMNM motif consists of the amino acid sequence set forth in YMNV (SEQ ID NO: 81), YENM (SEQ ID NO: 82), YMNQ (SEQ ID NO: 83), YMNL (SEQ ID NO: 84), or YSNM (SEQ ID NO: 85).

In certain embodiments, the mutated YMNM motif is a non-functional mutant. In certain embodiments, the mutated YMNM motif consists of the amino acid sequence Ybxb (SEQ ID NO: 86), wherein x is not an asparagine (N), and b is not a methionine (M). In certain embodiments, x is selected from the group consisting of A, R, D, C, E, Q, G, H, I, K, M, F, P, S, T, W, Y, V, and L. In certain embodiments, b is selected from the group consisting of A, R, N, D, C, E, Q, G, H, I, K, F, P, S, T, W, Y, V, and L. In certain embodiments, the mutated YMNM motif consists of the amino acid sequence set forth in YGGG (SEQ ID NO: 87), YAAA (SEQ ID NO: 88), YFFF (SEQ ID NO: 89), YETV (SEQ ID NO: 90), YQQQ (SEQ ID NO: 91), YHAE (SEQ ID NO: 92), YLDL (SEQ ID NO: 93), YLIP (SEQ ID NO: 94), YLRV (SEQ ID NO: 95), YTAV (SEQ ID NO: 96), or YVHV (SEQ ID NO: 97). In certain embodiments, the mutated YMNM motif consists of the amino acid sequence set forth in YGGG (SEQ ID NO: 87).

In certain embodiments, the intracellular signaling domain of the presently disclosed chimeric receptor comprises a co-stimulatory signaling domain that comprises a CD28 polypeptide comprising a mutated YMNM motif consisting of the amino acid sequence set forth in YENV (SEQ ID NO: 50), wherein the CD28 polypeptide comprises or consists of the amino acid sequence set forth in SEQ ID NO: 98. SEQ ID NO: 98 is provided below.

[SEQ ID NO: 98]
RSKRSRLLHSDYENVTPRRPGPTRKHYQPYAPPRDFAAYRS

In certain embodiments, the intracellular signaling domain of the presently disclosed chimeric receptor comprises a co-stimulatory signaling domain that comprises a CD28 polypeptide comprising a mutated YMNM motif consisting of the amino acid sequence set forth in YKNI (SEQ ID NO: 54), wherein the CD28 polypeptide comprises or consists of the amino acid sequence set forth in SEQ ID NO: 99. SEQ ID NO: 99 is provided below.

[SEQ ID NO: 99]
RSKRSRLLHSDYKNITPRRPGPTRKHYQPYAPPRDFAAYRS

In certain embodiments, the intracellular signaling domain of the presently disclosed chimeric receptor comprises a co-stimulatory signaling domain that comprises a CD28 polypeptide comprising a mutated YMNM motif consisting of the amino acid sequence set forth in YMDM (SEQ ID NO: 66), wherein the CD28 polypeptide comprises or consists of the amino acid sequence set forth in SEQ ID NO: 100. SEQ ID NO: 100 is provided below.

[SEQ ID NO: 100]
RSKRSRLLHSDYMDMTPRRPGPTRKHYQPYAPPRDFAAYRS

In certain embodiments, the intracellular signaling domain of the presently disclosed chimeric receptor comprises a co-stimulatory signaling domain that comprises a CD28 polypeptide comprising a mutated YMNM motif consisting of the amino acid sequence set forth in YGGG (SEQ ID NO: 87), wherein the CD28 polypeptide comprises or consists of the amino acid sequence set forth in SEQ ID NO: 101. SEQ ID NO: 101 is provided below.

[SEQ ID NO: 101]
RSKRSRLLHSDYGGGTPRRPGPTRKHYQPYAPPRDFAAYRS

In certain embodiments, the intracellular signaling domain of the presently disclosed chimeric receptor comprises a co-stimulatory signaling domain that comprises a CD28 polypeptide comprising a mutated YMNM motif consisting of the amino acid sequence set forth in YSNV (SEQ ID NO: 51), wherein the CD28 polypeptide comprises or consists of the amino acid sequence set forth in SEQ ID NO: 102. SEQ ID NO: 102 is provided below.

[SEQ ID NO: 102]
RSKRSRLLHSDYSNVTPRRPGPTRKHYQPYAPPRDFAAYRS

In certain embodiments, the intracellular signaling domain of the presently disclosed CAR comprises a first co-stimulatory signaling domain that comprises a CD28 polypeptide comprising a mutated YMNM motif (as disclosed herein), and a second co-stimulatory signaling domain that comprises an intracellular domain of a co-stimulatory molecule. Additional information regarding CARs including CD28 polypeptide comprising a mutated YMNM motif can be found in International Patent Publication No. WO 2021/158850, which is incorporated by reference in its entirety.

In certain embodiments, the intracellular signaling domain of the CAR comprises a co-stimulatory signaling region that comprises a 4-1BB polypeptide, e.g., an intracellular domain of 4-1BB or a fragment thereof. In certain embodiments, the intracellular signaling domain of the CAR comprises a co-stimulatory signaling region that comprises a 4-1BB polypeptide, e.g., an intracellular domain of human 4-1BB or a fragment thereof. In certain embodiments, the 4-1BB polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Ref. No.: NP_001552 (SEQ ID NO: 23) or a fragment thereof, and/or may optionally comprise up to one or up to two or up to three conservative amino acid substitutions. In non-limiting certain embodiments, the 4-1BB polypeptide comprises or consists of an amino acid sequence that is a consecutive portion of SEQ ID NO: 23, which is at least 20, or at least 30, or at least 40, or at least 50, or at least 100, or at least 150, or at least 150, and up to 255 amino acids in length. In certain embodiments, the 4-1BB polypeptide comprises or consists of an amino acid sequence of amino acids 1 to 255, 1 to 50, 50 to 100, 100 to 150, 150 to 200, or 200 to 255 of SEQ ID NO: 23. In certain embodiments, the intracellular signaling domain of the CAR comprises a co-stimulatory signaling region that comprises a 4-1BB polypeptide comprising or consisting of an amino acid sequence of amino acids 214 to 255 of SEQ ID NO: 23.

An exemplary nucleotide sequence encoding amino acids 214 to 255 of SEQ ID NO: 23 is set forth in SEQ ID NO: 24, which is provided below.

[SEQ ID NO: 24]
AAACGGGGCAGAAAGAAACTCCTGTATATATTCAAACAACCATTTATGA
GACCAGTACAAACTACTCAAGAGGAAGATGGCTGTAGCTGCCGATTTCC
AGAAGAAGAAGAAGGAGGATGTGAACTG

In certain embodiments, the intracellular signaling domain of the CAR comprises a co-stimulatory signaling region that comprises intracellular domains of two or more co-stimulatory molecules or portions thereof, e.g., an intracellular domain of CD28 or a fragment thereof and an intracellular domain of 4-1BB or a fragment thereof, or an intracellular domain of CD28 or a fragment thereof and an intracellular domain of OX40 or a fragment thereof.

In certain embodiments, the intracellular polypeptide of the chimeric receptor (e.g., CAR-DEL disclosed in Section 2.1) and the intracellular domain of the CAR are different.

### 2.4.2. TCR-Like Fusion Molecules

In certain embodiments, the antigen-recognizing receptor is a TCR like fusion molecule. Non-limiting examples of TCR fusion molecules include HLA-Independent TCR-based Chimeric Antigen Receptor (also known as “HIT-CAR”, e.g., those disclosed in International Patent Application No. PCT/US19/017525, which is incorporated by reference in its entirety), T cell receptor fusion constructs (TruCs) (e.g., those disclosed in Baeuerle et al., “Synthetic TruC receptors engaging the complete T cell receptor for potent anti-tumor response,” Nature Communications volume 10, Article number: 2087 (2019), which is incorporated by reference in its entirety), and T cell antigen coupler (TAC)s (which are chimeric receptors that co-opt the endogenous TCR) (e.g., those disclosed in Helsen et al., “The chimeric TAC receptor co-opts the T cell receptor yielding robust anti-tumor activity without toxicity,” Nature Communications (2018); 9:3049 (2018), which is incorporated by reference in its entirety).

In certain embodiments, the TCR like fusion molecule comprises an antigen binding chain that comprises an extracellular antigen-binding domain and a constant domain, wherein the TCR like fusion molecule binds to an antigen in an HLA-independent manner. In certain embodiments, the constant domain comprises a T cell receptor constant region selected from the group consisting of a native or modified TRAC peptide, a native or modified TRBC peptide, a native or modified TRDC peptide, a native or modified TRGC peptide and any variants or functional fragments thereof. In certain embodiments, the constant domain comprises a native or modified TRAC peptide. In certain embodiments, the constant domain comprises a native or modified TRBC peptide. In certain embodiments, the constant domain is capable of forming a homodimer or a heterodimer with another constant domain. In certain embodiments, the antigen binding chain is capable of associating with a CD33 polypeptide. In certain embodiments, the antigen binding chain, upon binding to an antigen, is capable of activating the CD35 polypeptide associated to the antigen binding chain. In certain embodiments, the activation of the CD32 polypeptide is capable of activating an immunoresponsive cell. In certain embodiments, the TCR like fusion molecule is capable of integrating with a CD3 complex and providing HLA-independent antigen recognition. In certain embodiments, the TCR like fusion molecule replaces an endogenous TCR in a CD3/TCR complex. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a ligand for a cell-surface receptor, a receptor for a cell surface ligand, an antigen binding portion of an antibody or a fragment thereof or an antigen binding portion of a TCR. In certain embodiments, the extracellular antigen-binding domain of the TCR like fusion molecule comprises one or two immunoglobulin variable region(s). In certain embodiments, the extracellular antigen-binding domain of the TCR like fusion molecule comprises a heavy chain variable region (V_{H}) of an antibody. In certain embodiments, the extracellular antigen-binding domain of the TCR like fusion molecule comprises a light chain variable region (V_{L}) of an antibody. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a V_{H} of an antibody, wherein the V_{H} is capable of dimerizing with another extracellular antigen-binding domain comprising a V_{L} of the antibody and form a fragment variable (Fv). In certain embodiments, the extracellular antigen-binding domain of the TCR like fusion molecule comprises a V_{L} of an antibody, wherein the V_{L} is capable of dimerizing with another extracellular antigen-binding domain comprising a V_{H} of the antibody and form a fragment variable (Fv).

### 2.4.3. T Cell Receptors (TCRs)

In certain embodiments, the second antigen-recognizing receptor is a T cell receptor (TCR). A TCR is a disulfide-linked heterodimeric protein consisting of two variable chains expressed as part of a complex with the invariant CD3 chain molecules. A TCR is found on the surface of T cells, and is responsible for recognizing antigens as peptides bound to major histocompatibility complex (MHC) molecules. In certain embodiments, a TCR comprises an alpha chain and a beta chain (encoded by TRA and TRB, respectively). In certain embodiments, a TCR comprises a gamma chain and a delta chain (encoded by TRG and TRD, respectively).

Each chain of a TCR is composed of two extracellular domains: Variable (V) region and a Constant (C) region. The Constant region is proximal to the cell membrane, followed by a transmembrane region and a short cytoplasmic tail. The variable region binds to the peptide/MHC complex. The variable domain of both chains each has three complementarity determining regions (CDRs).

In certain embodiments, a TCR can form a receptor complex with three dimeric signaling modules CD3δ/ε, CD3γ/ε and CD247 ζ/ζ or ζ/n. When a TCR complex engages with its antigen and MHC (peptide/MHC), the T cell expressing the TCR complex is activated.

In certain embodiments, the TCR is an endogenous TCR. In certain embodiments, the TCR is naturally occurring TCR.

In certain embodiments, the TCR is an exogenous TCR. In certain embodiments, the TCR is a recombinant TCR. In certain embodiments, the TCR is a non-naturally occurring TCR. In certain embodiments, the non-naturally occurring TCR differs from any naturally occurring TCR by at least one amino acid residue. In certain embodiments, the non-naturally occurring TCR differs from any naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues. In certain embodiments, the non-naturally occurring TCR is modified from a naturally occurring TCR by at least one amino acid residue. In certain embodiments, the non-naturally occurring TCR is modified from a naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues.

### 2.4.5. Chimeric Ligand Receptors

In certain embodiments, the antigen-recognizing receptor is a chimeric ligand receptor that comprises a ligand or a portion thereof that binds to the antigen. In certain embodiments, the chimeric ligand receptor further comprises a transmembrane domain and an intracellular signaling domain.

In certain embodiments, the transmembrane domain is fused to the ligand or portion thereof. In certain embodiments, the transmembrane domain is fused to the intracellular signaling domain. In certain embodiments, the transmembrane domain is positioned between the ligand or portion thereof and the intracellular signaling domain. In certain embodiments the transmembrane domain of the chimeric ligand receptor is a transmembrane domain disclosed in Section 2.4.1.1. In certain embodiments, the intracellular signaling domain of the chimeric ligand receptor comprises a CD32 polypeptide (e.g., as disclosed in Section 2.4.1.2).

Additional information on the presently disclosed chimeric ligand receptor can be found in Sauer et al., Blood (2021) 138 (4): 318-330, the content of which is incorporated by reference in its entirety.

### 2.4.6. CCRs

In certain embodiments, the antigen-recognizing receptor is a CCR. The term “chimeric co-stimulating receptor” or “CCR” refers to a chimeric receptor that binds to an antigen and provides a co-stimulatory signal, but does not provide activation signal to a cell comprising the CCR. Various CCRs are described in US20020018783 the contents of which are incorporated by reference in their entireties. CCRs mimic co-stimulatory signals, but unlike, CARs, do not provide activation signal. In certain embodiments, the CCR lacks a CD3ζ polypeptide.

CCRs provide co-stimulation signal (e.g., a CD28-like signal or 4-1BB-like signal), in the absence of the natural co-stimulatory ligand on the antigen-presenting cell. A combinatorial antigen recognition, i.e., use of a CCR in combination with a CAR, can augment immunoresponsive cell reactivity against the dual-antigen expressing cells, thereby improving selective tumor targeting. Kloss et al., describe a strategy that integrates combinatorial antigen recognition, split signaling, and, critically, balanced strength of T-cell activation and co-stimulation to generate T cells that eliminate target cells that express a combination of antigens while sparing cells that express each antigen individually (Kloss et al., Nature Biotechnology (2013); 31 (1): 71-75, the content of which is incorporated by reference in its entirety). With this approach, T-cell activation requires CAR-mediated recognition of one antigen, whereas co-stimulation is independently mediated by a CCR specific for a second antigen. To achieve tumor selectivity, the combinatorial antigen recognition approach diminishes the efficiency of T-cell activation to a level where it is ineffective without rescue provided by simultaneous CCR recognition of the second antigen.

In certain embodiments, the CCR comprises an extracellular antigen-binding domain that binds to a second antigen and an intracellular domain that is capable of delivering a costimulatory signal to the cell but does not alone deliver an activation signal to the cell. In certain embodiments, the CCR further comprises a transmembrane domain. In certain embodiments, the intracellular domain of the CCR comprises at least an intracellular domain of a co-stimulatory molecule or a portion thereof. In certain embodiments, the co-stimulatory molecule is selected from the group consisting of CD28, 4-1BB, OX40, CD27, CD40, CD154, CD97, CD11a/CD18, ICOS, DAP-10, CD2, CD150, CD226, and NKG2D.

In certain embodiments, the CCR comprises an intracellular domain of CD28 or a portion thereof. In certain embodiments, the CCR comprises an intracellular domain of 4-1BB or a portion thereof. In certain embodiments, the CCR comprises an intracellular domain of CD28 or a portion thereof, and an intracellular domain of 4-1BB or a portion thereof.

In certain embodiments, the second antigen is selected so that expression of both the first antigen and the second antigen is restricted to the targeted cells (e.g., cancerous tissue or cancerous cells, LSCs, or AML HSPCs). Similar to a CAR, the extracellular antigen-binding domain can be an scFv, a Fab, an F(ab) 2, or a chimeric protein with a heterologous sequence to form the extracellular antigen-binding domain.

In certain embodiments, the cell comprises a CAR-DEL, a first antigen-recognizing receptor (e.g., a CAR, a TCR, or a TCR-like fusion molecule), and a CCR. In certain embodiments, a cell comprising a first antigen-recognizing receptor (e.g., a CAR, a TCR, or a TCR-like fusion molecule) and a CCR exhibits a greater degree of cytolytic activity against cells that are positive for both the first antigen and the second antigen as compared to against cells that are singly positive for the first antigen. In certain embodiments, the cell comprising the first antigen-recognizing receptor and the CCR exhibits substantially no or negligible cytolytic activity against cells that are singly positive for the first antigen.

In certain embodiments, the first antigen recognizing receptor binds to the first antigen with a low binding affinity, e.g., a dissociation constant (K_{D}) of about 1×10⁻⁸ M or more, about 5×10⁻⁸ M or more, about 1×10⁻⁷ M or more, about 5×10⁻⁷ M or more, or about 1×10⁻⁶ M or more, or from about 1×10⁻⁸ M to about 1×10⁻⁶ M. In certain embodiments, the antigen recognizing receptor (e.g., a CAR, a TCR, or a TCR-like fusion molecule) binds to the antigen with a low binding avidity. In certain embodiments, the antigen recognizing receptor (e.g., a CAR, a TCR, or a TCR-like fusion molecule) binds to the antigen at an epitope of low accessibility. In certain embodiments, the antigen recognizing receptor (e.g., a CAR, a TCR, or a TCR-like fusion molecule) binds to the antigen with a binding affinity that is lower compared to the binding affinity with which the CCR binds to the second antigen. In certain embodiments, the CCR binds to the third antigen with a binding affinity K_{D} of from about 1×10⁻⁹ M to about 1×10⁻⁷ M, e.g., about 1×10⁻⁷ M or less, about 1×10⁻⁸ M or less, or about 1×10⁻⁹ M or less.

### 2.4.7. Delivery of the Antigen-Recognizing Receptors

In certain embodiments, the antigen-recognizing receptor or a polynucleotide encoding the same can be delivered to the cell by a viral method. In certain embodiments, the viral method comprises a viral vector. In certain embodiments, the viral vector is a retroviral vector (e.g., a gammaretroviral vector or a lentiviral vector). Other viral vectors include adenoviral vectors, adeno-associated viral vectors, vaccinia viruses, bovine papilloma viruses, and herpes viruses (e.g., such as Epstein-Barr Virus).

In certain embodiments, the antigen-recognizing receptor or a polynucleotide encoding the same can be delivered to the cell by a non-viral method. Any targeted genome editing methods can also be used to deliver the second antigen-recognizing receptor to the cell. In certain embodiments, the antigen-recognizing receptor and/or the chimeric receptor is delivered to the cell by a method comprising homologous recombination, a Zinc finger nuclease, a meganuclease, a Transcription activator-like effector nuclease (TALEN), a Clustered regularly-interspaced short palindromic repeats (CRISPR) system, or a combination thereof. In certain embodiments, a CRISPR system is used to deliver the second antigen-recognizing receptor to the cell.

In certain embodiments, the cell is a NK cell, and the antigen-recognizing receptor, or a polynucleotide encoding the same, is integrated at a locus within the genome of the NK cell.

Additionally or alternatively, the antigen-recognizing receptor and/or the chimeric receptor, or a polynucleotide encoding the same, can be integrated at a genomic safe harbor within the genome of the NK cell. Further information on genomic safe harbors and on methods for identifying the same can be found in International Patent Publications No. 2021/055592 and No. 2021/055616, the contents of each of which are incorporated by reference in their entirety.

### 2.5. Exemplified Cells

In certain embodiments, the presently disclosed cell is an immunoresponsive cell. In certain embodiments, the immunoresponsive cell comprises a chimeric receptor (e.g., CAR-DEL) disclosed herein.

In certain embodiments, the immunoresponsive cell is a NK cell. In certain embodiments, the NK cell comprises a chimeric receptor. In certain embodiments, the chimeric receptor binds to a first antigen and does not deliver activation and co-stimulatory signals to the cell. In certain embodiments, the chimeric receptor comprises (a) an extracellular antigen-binding domain comprising (i) a V_{H} that comprises a CDR1 comprising the amino acid sequence set forth in SEQ ID NO: 28, a CDR2 comprising the amino acid sequence set forth in SEQ ID NO: 29, and a CDR3 comprising the amino acid sequence set forth in SEQ ID NO: 30, and (ii) a V_{L} that comprises a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33; (b) a transmembrane domain comprising a CD28 polypeptide (e.g., a transmembrane domain of human CD28 or a fragment thereof), and (c) an intracellular polypeptide comprising a CD28 polypeptide. In certain embodiments, the transmembrane domain comprises a CD28 polypeptide that comprises amino acids 153 to 179 of SEQ ID NO: 40. In certain embodiments, the intracellular domain comprises a CD28 polypeptide comprising the amino acid sequence set amino acids 180 to 190 of SEQ ID NO: 40. In certain embodiments, the chimeric receptor comprises the amino acid sequence set forth in SEQ ID NO: 21 or SEQ ID NO: 22.

### 3. Nucleic Acids and Vectors

The presently disclosed subject matter provides nucleic acids comprising a polynucleotide encoding a chimeric receptor disclosed herein (e.g., disclosed in Section 2.1). In certain embodiments, the nucleic acids further comprise a polynucleotide encoding an antigen-recognizing receptor disclosed herein (e.g., disclosed in Section 2.3). Additionally or alternatively, the nucleic acids further comprise a polynucleotide encoding a cytokine disclosed herein (e.g., disclosed in Section 2.2). Also provided are cells comprising such nucleic acids. In certain embodiments, the nucleic acid further comprises a promoter that is operably linked to the chimeric receptor disclosed herein. In certain embodiments, the nucleic acid further comprises a second promoter that is operably linked to the antigen-recognizing receptor. Additionally or alternatively, the nucleic acid further comprises a third promoter that is operably linked to the cytokine.

In certain embodiments, at least one of the promoters is endogenous or exogenous.

In certain embodiments, the exogenous promoter is selected from an elongation factor (EF)-1 promoter, a CMV promoter, a SV40 promoter, a PGK promoter, and a metallothionein promoter. In certain embodiments, one or both of the first and second promoters are inducible promoters. In certain embodiments, the inducible promoter is selected from an NFAT transcriptional response element (TRE) promoter, a CD69 promoter, a CD25 promoter, and an IL-2 promoter.

In certain embodiments, the expression of the chimeric receptor is under the control of an endogenous promoter. In certain embodiments, the expression of the chimeric receptor and the antigen-recognizing receptor is under the control of an endogenous promoter. In certain embodiments, the expression of the chimeric receptor and the cytokine is under the control of an endogenous promoter. In certain embodiments, the expression of the chimeric receptor, the antigen-recognizing receptor, and the cytokine is under the control of an endogenous promoter.

In certain embodiments, the nucleic acid composition is a vector. In certain embodiments, the vector is a retroviral vector (e.g., a gammaretroviral vector or a lentiviral vector). In certain embodiments, the vector is viral vectors selected from the group consisting of adenoviral vectors, adena-associated viral vectors, vaccinia viruses, bovine papilloma viruses, and herpes viruses (e.g., such as Epstein-Barr Virus).

Additionally, the nucleic acids can be administered to subjects or and/delivered into cells by art-known methods or as described herein. Genetic modification of a cell (e.g., a T cell or an NK cell) can be accomplished by transducing a substantially homogeneous cell composition with a recombinant DNA construct. In certain embodiments, a retroviral vector (either gammaretroviral or lentiviral) is employed for the introduction of the nucleic acid compositions into the cell. For example, the first polynucleotide and the second polynucleotide can be cloned into a retroviral vector and expression can be driven from its endogenous promoter, from the retroviral long terminal repeat, or from a promoter specific for a target cell type of interest. Non-viral vectors may be used as well.

In certain embodiments, when the nucleic acid includes multiple polynucleotides, the polynucleotides can be constructed in a single, multicistronic expression cassette, in multiple expression cassettes of a single vector, or multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES, and encephalomyocarditis virus IRES) and cleavable linkers (e.g., 2A polypeptides, e.g., P2A, T2A, E2A and F2A polypeptides). Combinations of retroviral vectors and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114, or GALV envelope and any other known in the art.

In certain embodiments, the nucleic acid comprises a 2A polypeptide. For example, but without any limitation, when the nucleic acid includes a first polypeptide encoding a chimeric receptor (e.g., one disclosed in Section 2.1) and a second polypeptide encoding an antigen recognizing receptor (e.g., one disclosed in Section 2.3), the first polypeptide and the second polypeptide can be linked by a 2A polypeptide. In certain embodiments, the 2A polypeptide can be a P2A polypeptide, a T2A polypeptide, a E2A polypeptide, or a F2A polypeptide. In certain embodiments, the P2A polypeptide comprises or consists of the amino acid sequence set forth in SEQ ID NO: 17. In certain embodiments, the P2A polypeptide comprises or consists of the amino acid sequence set forth in SEQ ID NO: 18. SEQ ID NO: 17 and SEQ ID NO: 18 are provided below:

[SEQ ID NO: 17]
ATNFSLLKQAGDVEENPGP
[SEQ ID NO: 18]
EGRGSLLTCGDVEENPGP

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic modification of a cell. For example, a nucleic acid molecule can be delivered into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Transient expression may be obtained by RNA electroporation.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but are not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

In certain embodiments, the delivery methods include the use of colloids. As used herein, the term “colloid” refers to systems in which there are two or more phases, with one phase (e.g., the dispersed phase) distributed in the other phase (e.g., the continuous phase). Moreover, at least one of the phases has small dimensions (in the range of about 10-9 to about 106 m). Non-limiting examples of colloids encompassed by the presently disclosed subject matter include macromolecule complexes, nanocapsules, microspheres, beads, and lipid-based systems (e.g., micelles, liposomes, and lipid nanoparticles).

In certain embodiments, the delivery methods include the use of liposomes. The term “liposome,” as used herein, refers to single- or multi-layered spherical lipid bilayer structures produced from lipids dissolved in organic solvents and then dispersed in aqueous media. Experimentally and therapeutically used for delivering an active pharmaceutical ingredient (e.g., nucleic acid compositions disclosed herein) to cells, liposomes fuse with cell membranes so the contents are transferred into the cytoplasm.

In certain embodiments, the delivery methods include the use of lipid nanoparticles. As used herein, the term “lipid nanoparticle” refers to a particle having at least one dimension in the order of nanometers (e.g., from about 1 nm to about 1,000 nm) and including at least one lipid. In certain embodiments, the lipid nanoparticles can include an active pharmaceutical ingredient (e.g., nucleic acid compositions disclosed herein) for delivering to cells. The morphology of the lipid nanoparticles can be different from liposomes. While liposomes are characterized by a lipid bilayer surrounding a hydrophilic core, lipid nanoparticles have an electron-dense core where cationic lipids and/or ionizable lipids are organized into inverted micelles around an active pharmaceutical ingredient (e.g., nucleic acid compositions disclosed herein). Additional information on the morphology and properties of lipid nanoparticles and liposomes can be found in Wilczewska, et al., Pharmacological reports 64, no. 5 (2012): 1020-1037; Eygeris et al., Accounts of Chemical Research 55, no. 1 (2021): 2-12; Zhang et al., Chemical Reviews 121, no. 20 (2021): 12181-12277; and Fan et al., Journal of pharmaceutical and biomedical analysis 192 (2021): 113642.

In certain embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

In certain embodiments, the lipid nanoparticles can include a cationic lipid or an ionizable lipid. The term “cationic lipid” refers to lipids including a head group with permanent positive charges. Non-limiting examples of cationic lipids encompassed by the presently disclosed subject matter include 1,2-di-O-octadecenyl-3-trimethylammonium-propane (DOTMA), 1,2-dioleoyl-3-trimethylammonium-propane (DOTAP), 2,3-dioleyloxy-N-[2-(sperminecarboxamido)ethyl]-N,N-dimethyl-1-propanaminium trifluoroacetate (DOSPA), and ethylphosphatidylcholine (ePC).

As used herein, the term “ionizable lipid” refers to lipids that are protonated at low pH and are neutral at physiological pH. The pH-sensitivity of ionizable lipids is particularly beneficial for delivery in vivo (e.g., delivery of nucleic acid compositions disclosed herein), because neutral lipids have less interactions with the anionic membranes of blood cells and, thus, improve the biocompatibility of the lipid nanoparticles. Once trapped in endosomes, ionizable lipids are protonated and promote membrane destabilization to allow the endosomal escape of the nanoparticles. Non-limiting example of ionizable lipids encompassed by the presently disclosed subject matter include tetrakis(8-methylnonyl) 3,3′,3″,3″-(((methylazanediyl)bis(propane-3,1 diyl))bis(azanetriyl))tetrapropionate; decyl(2-(dioctylammonio)ethyl) phosphate; ((4-hydroxybutyl) azanediyl)bis(hexane-6,1-diyl)bis(2-hexyldecanoate); bis(2-(dodecyldisulfanyl)ethyl) 3,3′-((3-methyl-9-oxo-10-oxa-13,14-dithia-3,6-diazahexacosyl) azanediyl)dipropionate; 1,1′-((2-(4-(2-((2-(bis(2-hydroxydodecyl)amino)ethyl) (2-hydroxydodecyl)amino)ethyl) piperazin-1-yl)ethyl) azanediyl)bis(dodecan-2-ol); cKK-E12, 3,6-bis(4-(bis(2-hydroxydodecyl)amino)butyl) piperazine-2,5-dione; (6Z,9Z,28Z,31Z)-heptatriaconta-6,9,28,31-tetraen-19-yl 4-(dimethylamino)butanoate; hexa (octan-3-yl) 9,9′,9″,9″,9″,9″-((((benzene-1,3,5-tricarbonyl)yris(azanediyl)) tris (propane-3,1-diyl)) tris(azanetriyl)) hexanonanoate; heptadecan-9-yl 8-((2-hydroxyethyl) (6-oxo-6-(undecyloxy) hexyl)amino) octanoate; and (((3,6-dioxopiperazine-2,5-diyl)bis(butane-4, 1-diyl))bis(azanetriyl))tetrakis(ethane-2,1-diyl) (9Z,9′Z,9″Z,9″Z,12Z,12′Z,12″Z,12″Z)-tetrakis (octadeca-9,12-dienoate).

Additionally, in certain embodiments, the lipid nanoparticles can include other lipids. For example, but without any limitation, the lipid nanoparticles of the presently disclosed subject matter can include phospholipids, cholesterol, polyethylene glycol (PEG)-functionalized lipids (PEG-lipids). These lipids can improve certain properties of the lipid nanoparticles (e.g., stability, biodistribution, etc.). For example, cholesterol enhances the stability of the lipid nanoparticles by modulating their integrity and rigidity. Non-limiting examples of other lipids present in lipid nanoparticles include cholesterol, DC-cholesterol, β-sitosterol, BHEM-cholesterol, ALC-0159, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoylphosphatidylethanolamine (DSPE), 16-O-monomethyl PE, 16-O-dimethyl PE, 18-1-trans PE, 1-stearioyl-2-oleoyl-phosphatidyethanol amine (SOPE), and 1,2-dielaidoyl-sn-glycero-3-phophoethanolamine (transDOPE).

In certain embodiments, the lipid nanoparticles can include a targeting moiety that binds to a ligand. The use of the targeting moieties allows selective delivery of an active pharmaceutical ingredient (e.g., nucleic acid compositions disclosed herein) to target cells expressing the ligand (e.g., NK cells). In certain embodiments, the targeting moiety can be an antibody or antigen-binding fragment thereof that binds to a cell surface receptor. For example, but without any limitation, the targeting domain is an antibody or antigen-binding fragment thereof that binds to a receptor expressed on the surface of a cell (e.g., CD3, CD4, CD8, CD16, CD40L, CD95, FasL, CTLA-4, OX40, GITR, LAG3, ICOS, and PD-1).

In certain embodiments, the delivery methods are in vivo delivery methods. In certain embodiments, the delivery methods are ex vivo delivery methods.

### 4. Polypeptides

The presently disclosed subject matter provides methods for evaluating an amino acid sequence or a nucleotide sequence by producing an alteration in the sequence. Such alterations may include certain mutations, deletions, insertions, or post-translational modifications. The presently disclosed subject matter further includes analogs of any naturally-occurring polypeptides disclosed herein (including, but not limited to, CD33, CD8, CD28, 4-1BB, and CD3ζ,). Analogs can differ from a naturally-occurring polypeptide disclosed herein by amino acid sequence differences, by post-translational modifications, or by both. Analogs can exhibit at least about 85%, about 90%, about 91%, about 92%, about 93%, about 94%, about 95%, about 96%, about 97%, about 98%, about 99% or more homologous or identical to all or part of a naturally-occurring amino, acid sequence of the presently disclosed subject matter. The length of sequence comparison is at least 5, 10, 15 or 20 amino acid residues, e.g., at least 25, 50, or 75 amino acid residues, or more than 100 amino acid residues. Again, in an exemplary approach to determining the degree of identity, a BLAST program may be used, with a probability score between e-3 and e-100 indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., β or γ amino acids.

In addition to full-length polypeptides, the presently disclosed subject matter also provides fragments of any of the polypeptides disclosed herein. As used herein, the term “a fragment” means at least 5, 10, 13, or 15 amino acids. In certain embodiments, a fragment comprises at least 20 contiguous amino acids, at least 30 contiguous amino acids, or at least 50 contiguous amino acids. In certain embodiments, a fragment comprises at least 60 to 80, 100, 200, 300 or more contiguous amino acids. Fragments can be generated by methods known to those skilled in the art or may result from normal protein processing (e.g., removal of amino acids from the nascent polypeptide that are not required for biological activity or removal of amino acids by alternative mRNA splicing or alternative protein processing events).

### 5. Formulations and Administration

The presently disclosed subject matter provides compositions comprising presently disclosed cells (e.g., disclosed in Section 2). Additionally, the presently disclosed subject matter provides compositions comprising presently disclosed nucleic acids (e.g., disclosed in Section 3). In certain embodiments, the compositions are pharmaceutical compositions that further comprise a pharmaceutically acceptable excipient.

Compositions comprising the presently disclosed cells and/or nucleic acids can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate-buffered saline, polyol (for example, glycerol, propylene glycol, liquid polyethylene glycol, and the like), and suitable mixtures thereof.

Compositions comprising the presently disclosed cells and/or nucleic acids can be provided systemically or directly to a subject for inducing and/or enhancing an immune response to an antigen and/or treating and/or preventing a neoplasm. In certain embodiments, the presently disclosed cells, nucleic acids, or compositions comprising thereof are directly injected into an organ of interest (e.g., an organ affected by a neoplasm). Alternatively, the presently disclosed cells, nucleic acids, or compositions comprising thereof are provided indirectly to the organ of interest, for example, by administration into the circulatory system (e.g., the tumor vasculature). Expansion and differentiation agents can be provided prior to, during, or after administration of the cells or compositions to increase the production of cells in vitro or in vivo.

The number of cells to be administered can vary for the subject being treated. In certain embodiments, between about 10⁴ and about 10¹⁰, between about 10⁴ and about 10⁷, between about 10⁵ and about 10⁷, between about 10⁵ and about 10⁹, or between about 10⁶ and about 10⁸ of the presently disclosed cells are administered to a subject. In certain embodiments, between about 10⁵ and about 10⁷ of the presently disclosed cells are administered to a subject. More effective cells may be administered in even smaller numbers. Usually, at least about 1×10⁵ cells will be administered, eventually reaching about 1×10¹⁰ or more. In certain embodiments, at least about 1×10⁵, about 5×10⁵, about 1×10⁶, about 5×10⁶, about 1×10⁷, about 5×10⁷, about 1×10⁸, or about 5×10⁸ of the presently disclosed cells are administered to a subject. In certain embodiments, about 1×10⁵ of the presently disclosed cells are administered to a subject. In certain embodiments, about 5×10⁵ of the presently disclosed cells are administered to a subject. In certain embodiments, about 1×10⁶ of the presently disclosed cells are administered to a subject. The precise determination of what would be considered an effective dose can be based on factors individual to each subject, including the size, age, sex, weight, and condition of the particular subject. Dosages can be readily ascertained by those skilled in the art from this disclosure and the knowledge in the art.

The presently disclosed cells and compositions can be administered by any method known in the art including, but not limited to, intravenous administration, subcutaneous administration, intranodal administration, intratumoral administration, intrathecal administration, intrapleural administration, intraosseous administration, intraperitoneal administration, pleural administration, and direct administration to the subject. The presently disclosed cells can be administered in any physiologically acceptable vehicle, normally intravascularly, although they may also be introduced into bone or other convenient sites where the cells may find an appropriate site for regeneration and differentiation (e.g., thymus). The cells can be introduced by injection, catheter, or the like.

Compositions comprising the presently disclosed cells and/or nucleic acids can be provided systemically or directly to a subject for inducing and/or enhancing an immune response to an antigen and/or treating and/or preventing a neoplasm (e.g., cancer), pathogen infection, or infectious disease. In certain embodiments, the presently disclosed cells, nucleic acids, or compositions thereof are directly injected into an organ of interest (e.g., an organ affected by a neoplasm). Alternatively, the presently disclosed cells, nucleic acids, or compositions thereof are provided indirectly to the organ of interest, for example, by administration into the circulatory system (e.g., the tumor vasculature). Expansion and differentiation agents can be provided prior to, during, or after administration of the cells, compositions, or nucleic acid compositions to increase production of the cells (e.g., T cells (e.g., CTL cells) or NK cells) in vitro or in vivo.

The presently disclosed compositions can be pharmaceutical compositions comprising the presently disclosed cells or their progenitors and a pharmaceutically acceptable carrier. Administration can be autologous or heterologous. For example, cells, or progenitors can be obtained from one subject and administered to the same subject or a different, compatible subject. Peripheral blood derived cells or their progeny (e.g., in vivo, ex vivo, or in vitro derived) can be administered via localized injection, including catheter administration, systemic injection, localized injection, intravenous injection, or parenteral administration. When administering a therapeutic composition of the presently disclosed subject matter (e.g., a pharmaceutical composition comprising a presently disclosed cell), it can be formulated in a unit dosage injectable form (solution, suspension, emulsion).

### 6. Methods of Treatment

The presently disclosed subject matter provides various methods of using the presently disclosed cells, nucleic acids, or compositions thereof. The presently disclosed cells, nucleic acids, or compositions thereof can be used in therapy or medicament. For example, the presently disclosed subject matter provides methods for inducing and/or increasing an immune response in a subject in need thereof. The presently disclosed cells and compositions comprising thereof can be used for reducing tumor burden in a subject. The presently disclosed cells and compositions comprising thereof can reduce the number of tumor cells, reduce tumor size, and/or eradicate the tumor in the subject. The presently disclosed cells, nucleic acids, or compositions thereof can be used for treating and/or preventing a tumor (or neoplasm) in a subject. The presently disclosed cells, nucleic acids, or compositions thereof can be used for prolonging the survival of a subject suffering from a tumor. In certain embodiments, the tumor is cancer. The presently disclosed cells, nucleic acids, or compositions thereof can also be used for treating and/or preventing a pathogen infection or other infectious disease in a subject, such as an immunocompromised human subject. The presently disclosed cells, nucleic acids, or compositions thereof can also be used for treating and/or preventing an autoimmune disease in a subject. In certain embodiments, each of the above-noted methods comprises administering the presently disclosed cells, nucleic acids, or compositions thereof (e.g., a pharmaceutical composition comprising the cells or a pharmaceutical composition comprising the nucleic acids) to achieve the desired effect, e.g., palliation of an existing condition or prevention of recurrence. For treatment, the amount administered is an amount effective in producing the desired effect. An effective amount can be provided in one or a series of administrations. An effective amount can be provided in a bolus or by continuous perfusion.

Non-limiting examples of tumors (or neoplasms) include blood cancers (e.g. leukemias, lymphomas, and myelomas), ovarian cancer, breast cancer, bladder cancer, brain cancer, colon cancer, intestinal cancer, liver cancer, lung cancer, pancreatic cancer, prostate cancer, skin cancer, stomach cancer, glioblastoma, throat cancer, melanoma, neuroblastoma, adenocarcinoma, glioma, soft tissue sarcoma, and various carcinomas (including prostate and small cell lung cancer). Suitable carcinomas further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolar carcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. In certain embodiments, the neoplasm is cancer. In certain embodiments, the neoplasm is selected from the group consisting of blood cancers (e.g. leukemias, lymphomas, and myelomas), ovarian cancer, prostate cancer, breast cancer, bladder cancer, brain cancer, colon cancer, intestinal cancer, liver cancer, lung cancer, pancreatic cancer, prostate cancer, skin cancer, stomach cancer, glioblastoma, and throat cancer. In certain embodiments, the presently disclosed cells, nucleic acids, or compositions thereof can be used for treating and/or preventing blood cancers (e.g., leukemias, lymphomas, and myelomas) or ovarian cancer, which are not amenable to conventional therapeutic interventions.

In certain embodiments, the tumor and/or neoplasm is a solid tumor. Non-limiting examples of solid tumors include renal cell carcinoma, non-small-cell lung cancer, lung adenocarcinoma, lung squamous cell carcinoma, lung neuroendocrine carcinoma, small-cell lung cancer, pancreatic cancer, breast cancer, astrocytoma, glioblastoma, laryngeal/pharyngeal carcinoma, EBV-associated nasopharyngeal carcinoma, and ovarian carcinoma.

In certain embodiments, the tumor and/or neoplasm is a blood cancer. Non-limiting examples of blood cancer include multiple myeloma, leukemia, and lymphomas. Non-limiting examples of leukemia include acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, and B cell prolymphocytic leukemia. The lymphoma can be Hodgkin's lymphoma or non-Hodgkin's lymphoma. In certain embodiments, the lymphoma is non-Hodgkin's lymphoma, including B-cell non-Hodgkin's lymphoma and T-cell non-Hodgkin's lymphoma.

In certain embodiments, the tumor and/or neoplasm is a B cell malignancy. Non-limiting examples of B cell malignancy include B cell non-Hodgkin lymphomas (NHL), B cell Hodgkin's lymphomas, B cell acute lymphocytic leukemia (ALL), B cell chronic lymphocytic leukemia (CLL), multiple myeloma (MM), CLL with Richter's transformation, and CNS lymphoma.

In certain embodiments, the tumor and/or neoplasm is a B cell-related neoplasm. Non-limiting examples of B cell-related neoplasm include chronic lymphocytic leukemia/small lymphocytic lymphoma (CLL/SLL), B-cell prolymphocytic leukemia, splenic marginal zone lymphoma, hairy cell leukemia, splenic B-cell lymphoma/leukemia (unclassifiable), splenic diffuse red pulp small B-cell lymphoma, lymphoplasmacytic lymphoma, Waldenström macroglobulinemia, monoclonal gammopathy of undetermined significance (MGUS, IgM), heavy-chain diseases (μ, γ, α), MGUS (IgG/A), plasma cell myeloma, solitary plasmacytoma of bone, extraosseous plasmacytoma, monoclonal immunoglobulin deposition diseases, extranodal marginal zone lymphoma of mucosa-associated lymphoid tissue (MALT lymphoma), nodal marginal zone lymphoma, pediatric nodal marginal zone lymphoma, follicular lymphoma, in situ follicular neoplasia, duodenal-type follicular lymphoma, pediatric-type follicular lymphoma, large B-cell lymphoma with IRF4 rearrangement, primary cutaneous follicle center cell lymphoma, mantle cell lymphoma, in situ mantle cell neoplasia, diffuse large B-cell lymphoma (DLBCL) (not otherwise specified (NOS)), germinal center B-cell type, activated B-cell type, T-cell/histiocyte-rich large B-cell lymphoma, primary DLBCL of the central nervous system (CNS), primary cutaneous DLBCL (leg type), Epstein-Barr virus (EBV)-positive DLBCL (NOS), EBV-positive mucocutaneous ulcer, DLBCL associated with chronic inflammation, lymphomatoid granulomatosis, primary mediastinal (thymic) large B-cell lymphoma, intravascular large B-cell lymphoma, anaplastic lymphoma kinase (ALK)-positive large B-cell lymphoma, plasmablastic lymphoma, primary effusion lymphoma, human herpesvirus 8 (HHV-8)-associated DLBCL (NOS), Burkitt lymphoma, Burkitt-like lymphoma with 11q aberration, high-grade B-cell lymphoma with MYC and BLC2 and/or BCL6 rearrangements, high-grade B-cell lymphoma (NOS), and B-cell lymphoma (unclassifiable).

In certain embodiments, the tumor and/or neoplasm is a myeloid disorder. Non-limiting examples of myeloid disorders include myelodysplastic syndromes, myeloproliferative neoplasms, chronic myelomonocytic leukemia, acute myeloid leukemia (AML), blastic plasmacytoid dendritic cell neoplasm, acute myeloblastic leukemia, acute promyelocytic leukemia, acute myelomonocytic leukemia, chronic myelocytic leukemia, and polycythemia vera. In certain embodiments, the myeloid disorder is acute myeloid leukemia (AML).

The presently disclosed subject matter provides methods for treating and/or preventing a viral infection in a subject. The method can comprise administering an effective amount of the presently disclosed cells, nucleic acids, or compositions thereof to a subject having a viral infection. Non-limiting examples of viral infections include those caused by cytomegalovirus (CMV), Epstein-Barr virus (EBV), hepatitis A, B, C, D, E, F or G, human immunodeficiency virus (HIV), adenovirus, BK polyomavirus, coronavirus, coxsackievirus, poliovirus, herpes simplex type 1, herpes simplex type 2, human cytomegalovirus, human herpesvirus type 8, varicella-zoster virus, influenza virus, measles virus, mumps virus, parainfluenza virus, respiratory syncytial virus, papillomavirus, rabies virus, and Rubella virus. Other viral targets include Paramyxoviridae (e.g., pneumovirus, morbillivirus, metapneumovirus, respirovirus or rubulavirus), Adenoviridae (e.g., adenovirus), Arenaviridae (e.g., arenavirus such as lymphocytic choriomeningitis virus), Arteriviridae (e.g., porcine respiratory and reproductive syndrome virus or equine arteritis virus), Bunyaviridae (e.g., phlebovirus or hantavirus), Caliciviridae (e.g., Norwalk virus), Coronaviridae (e.g., coronavirus or torovirus), Filoviridae (e.g., Ebola-like viruses), Flaviviridae (e.g., hepacivirus or flavivirus), Herpesviridae (e.g., simplexvirus, varicellovirus, cytomegalovirus, roseolovirus, or lymphocryptovirus), Orthomyxoviridae (e.g., influenza virus or thogotovirus), Parvoviridae (e.g., parvovirus), Picomaviridae (e.g., enterovirus or hepatovirus), Poxviridae (e.g., orthopoxvirus, avipoxvirus, or leporipoxvirus), Retroviridae (e.g., lentivirus or spumavirus), Reoviridae (e.g., rotavirus), Rhabdoviridae (e.g., lyssavirus, novirhabdovirus, or vesiculovirus), and Togaviridae (e.g., alphavirus or rubivirus). In certain embodiments, the viral infections include human respiratory coronavirus, influenza viruses A-C, hepatitis viruses A to G, and herpes simplex viruses 1-9. In certain embodiments, the subject has an immunodeficiency.

The presently disclosed subject matter provides methods for treating and/or preventing a bacterial infection in a subject. The method can comprise administering an effective amount of the presently disclosed cells, nucleic acids, or compositions thereof to a subject having a bacterial infection. Bacterial infections include, but are not limited to, Mycobacteria, Rickettsia, Mycoplasma, Neisseria meningitides, Neisseria gonorrheoeae, Legionella, Vibrio cholerae, Streptococci, Staphylococcus aureus, Staphylococcus epidermidis, Pseudomonas aeruginosa, Corynobacteria diphtheriae, Clostridium spp., enterotoxigenic Escherichia coli, Bacillus anthracis, Rickettsia, Bartonella henselae, Bartonella quintana, Coxiella burnetii, chlamydia, Mycobacterium leprae, shigella, Salmonella, Yersinia enterocolitica, Yersinia pseudotuberculosis; Legionella pneumophila; Mycobacterium tuberculosis; Listeria monocytogenes; Mycoplasma spp., Pseudomonas fluorescens, Vibrio cholerae, Haemophilus influenzae, Bacillus anthracis, Treponema pallidum, Leptospira, Borrelia, Corynebacterium diphtheriae, Francisella, Brucella melitensis, Campylobacter jejuni, Enterobacter, Proteus mirabilis, Proteus, and Klebsiella pneumoniae.

The presently disclosed subject matter provides methods for treating and/or preventing an autoimmune disease in a subject. The method can comprise administering an effective amount of the presently disclosed cells, nucleic acids, or compositions thereof to a subject having an autoimmune disease.

The presently disclosed subject matter provides methods for treating and/or preventing an inflammatory disease in a subject. The method can comprise administering an effective amount of the presently disclosed cells, nucleic acids, or compositions thereof to a subject having an infectious disease.

Non-limiting examples of autoimmune diseases and inflammatory diseases or conditions thereof include arthritis, e.g., rheumatoid arthritis (RA), Type I diabetes, systemic lupus erythematosus (SLE), inflammatory bowel disease, ulcerative colitis, psoriasis, psoriatic arthritis, scleroderma, autoimmune thyroid disease, Grave's disease, Crohn's disease, multiple sclerosis, systemic sclerosis, asthma, organ transplant rejection, a disease or condition associated with transplant, Takayasu arteritis, giant-cell arteritis, Kawasaki disease, polyarteritis nodosa, Behcet's syndrome, Wegener's granulomatosis, ANCA-vasculitides, Churg-Strauss syndrome, microscopic polyangiitis, vasculitis of connective tissue diseases, Hennoch-Schonlein purpura, cryoglobulinemic vasculitis, cutaneous leukocytoclastic angiitis, Sarcoidosis, Cogan's syndrome, Wiskott-Aldrich Syndrome, primary angiitis of the CNS, thromboangiitis obliterans, paraneoplastic arteritis, myelodysplastic syndrome, erythema elevatum diutinum, amyloidosis, autoimmune myositis, Guillain-Barre Syndrome, histiocytosis, atopic dermatitis, pulmonary fibrosis, glomerulonephritis, Whipple's disease, Still's disease, Sjogren's syndrome, osteomyelofibrosis, chronic inflammatory demyelinating polyneuropathy, Kimura's disease, systemic sclerosis, chronic periaortitis, chronic prostatitis, idiopathic pulmonary fibrosis, chronic granulomatous disease, idiopathic, bleomycin-induced lung inflammation, cytarabine-induced lung inflammation, autoimmune thrombocytopenia, autoimmune neutropenia, autoimmune hemolytic anemia, autoimmune lymphocytopenia, chronic autoimmune thyroiditis, autoimmune hepatitis, Hashimoto's thyroiditis, atopic thyroiditis, Graves disease, autoimmune polyglandular syndrome, autoimmune Addison syndrome, and/or myasthenia gravis. In accordance with the presently disclosed subject matter, the above-described various methods can comprise administering to the subject a checkpoint immune blockade agent.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Further modification can be introduced to the presently disclosed cells to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the presently disclosed cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T-cell elimination by administering anti-EGFR monoclonal antibody (e.g., cetuximab). EGFRt can be covalently joined to the upstream of the chimeric receptor disclosed herein. The suicide gene can be included within the vector comprising nucleic acids encoding a presently disclosed chimeric receptor. In this way, administration of a prodrug designed to activate the suicide gene (e.g., a prodrug (e.g., AP1903 that can activate iCasp-9) during malignant T-cell transformation (e.g., GVHD) triggers apoptosis in the suicide gene-activated cells expressing the presently disclosed chimeric receptor. The incorporation of a suicide gene into a presently disclosed chimeric receptor gives an added level of safety with the ability to eliminate the majority of receptor-expressing cells within a very short time period. A presently disclosed cell incorporated with a suicide gene can be pre-emptively eliminated at a given timepoint post the cell infusion, or eradicated at the earliest signs of toxicity.

### 7. Kits

The presently disclosed subject matter provides kits for inducing and/or enhancing an immune response and/or treating and/or preventing a neoplasm or a pathogen infection (e.g., an autoimmune disease or an infectious disease) in a subject. In certain embodiments, the kit comprises an effective amount of presently disclosed cells, nucleic acids, or compositions thereof. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain non-limiting embodiments, the kit includes a nucleic acid molecule encoding the chimeric receptor disclosed herein. In certain non-limiting embodiments, the kit further includes a nucleic acid molecule encoding an antigen-recognizing receptor disclosed herein (e.g., a CAR, a TCR, or a TCR-like fusion molecule) directed toward an antigen of interest in expressible form, which may optionally be comprised in the same or different vectors.

If desired, the cells, nucleic acids, or compositions thereof are provided together with instructions for administering the cells, nucleic acids, or compositions thereof to a subject having or at risk of developing a tumor (e.g., a cancer) or a pathogen infection (e.g., an infectious disease), or immune disorder (e.g., an autoimmune disease). The instructions generally include information about the use of the cells, nucleic acids, or compositions thereof for the treatment and/or prevention of a neoplasm, or a pathogen infection (e.g., an infectious disease), or an immune disorder (e.g., an autoimmune disease). In certain embodiments, the instructions include at least one of the following: description of the therapeutic agent; dosage schedule and administration for treatment or prevention of a neoplasm, pathogen infection (e.g., an infectious disease), or immune disorder (e.g., an autoimmune disease) or symptoms thereof; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### 8. Exemplary Embodiments

Embodiment 1. An immunoresponsive cell comprising a chimeric receptor that binds to an antigen without delivering: (1) an activation signal; and/or (2) a co-stimulatory signal to the immunoresponsive cell.

Embodiment 2. The immunoresponsive cell of embodiment 1, wherein the chimeric receptor comprises an extracellular antigen-binding domain and a transmembrane domain.

Embodiment 3. The immunoresponsive cell of embodiment 1, wherein the chimeric receptor comprises an intracellular polypeptide.

Embodiment 4. The cell of embodiment 2 or 3, wherein the transmembrane domain comprises a CD8 polypeptide, a CD28 polypeptide, a CD32 polypeptide, a CD4 polypeptide, a 4-1BB polypeptide, an OX40 polypeptide, an ICOS polypeptide, a CD84 polypeptide, a CD166 polypeptide, a CD8a polypeptide, a CD8b polypeptide, a ICAM-1 polypeptide, a CTLA-4 polypeptide, a CD27 polypeptide, a CD40 polypeptide, a NKGD2 polypeptide, a PD-1 polypeptide, a LAG-3 polypeptide, a 2B4 polypeptide, a BTLA polypeptide, a DAP12 polypeptide, a DAP10 polypeptide, a CD16 polypeptide, a DNAM1 polypeptide, or a combination thereof.

Embodiment 5. The immunoresponsive cell of embodiment 3 or 4, wherein the intracellular polypeptide comprises a CD28 polypeptide, a CD4 polypeptide, a 4-1BB polypeptide, an OX40 polypeptide, an ICOS polypeptide, a CD84 polypeptide, a CD166 polypeptide, a CD8a polypeptide, a CD8b polypeptide, a ICAM-1 polypeptide, a CTLA-4 polypeptide, a CD27 polypeptide, a CD40 polypeptide, a NKGD2 polypeptide, a PD-1 polypeptide, a LAG-3 polypeptide, a 2B4 polypeptide, a BTLA4 polypeptide, a DAP12 polypeptide, a DAP-10 polypeptide, a CD16 polypeptide, a CD266 polypeptide, or a combination thereof.

Embodiment 6. The immunoresponsive cell of any one of embodiments 3-5, wherein the intracellular polypeptide comprises a CD28 polypeptide.

Embodiment 7. The immunoresponsive cell of any one of embodiments 3-6, wherein the intracellular polypeptide comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% identical to the amino acid sequence set forth in SEQ ID NO: 16.

Embodiment 8. The immunoresponsive cell of any one of embodiments 3-7, wherein the intracellular polypeptide comprises the amino acid sequence set forth in SEQ ID NO: 16.

Embodiment 9. The immunoresponsive cell of any one of embodiment 8, wherein the intracellular polypeptide consists of the amino acid sequence set forth in SEQ ID NO: 16.

Embodiment 10. The immunoresponsive cell of any one of embodiments 3-9, wherein the intracellular polypeptide lacks a CD32 polypeptide.

Embodiment 11. The immunoresponsive cell of any one of embodiments 1-10, wherein the antigen is a tumor antigen or a pathogen antigen.

Embodiment 12. The immunoresponsive cell of any one of embodiments 1-11, wherein the antigen is a tumor antigen.

Embodiment 13. The immunoresponsive cell of any one of embodiments 1-12, wherein the tumor antigen is selected from CD33, CD19, carbonic anhydrase IX (CAIX), carcinoembryonic antigen (CEA), CD8, CD7, CD10, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinases Erb-B2,3,4 (erb-B2,3,4), folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13R□2), □-light chain, kinase insert domain receptor (KDR), Lewis Y (LeY), L1 cell adhesion molecule (LICAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, NKCS1, EGF1R, EGFR-VIII, CD99, CD70, ADGRE2, CCR1, LILRB2, PRAME CCR4, CD5, CD3, TRBC1, TRBC2, TIM-3, Integrin B7, ICAM-1, CD70, Tim3, CLEC12A, or ERBB.

Embodiment 14. The cell of any one of embodiments 1-13, wherein the antigen is CD33.

Embodiment 15. The cell of embodiment 14, wherein the extracellular antigen-binding domain comprises a heavy chain variable region (VH) comprising a CDR1 comprising the amino acid sequence set forth in SEQ ID NO: 28, a CDR2 comprising the amino acid sequence set forth in SEQ ID NO: 29, and a CDR3 comprising the amino acid sequence set forth in SEQ ID NO: 30, and a light chain variable region (VL) comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33.

Embodiment 16. The immunoresponsive cell of any one of embodiments 1-15, wherein the chimeric receptor comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% identical to the amino acid sequence set forth in SEQ ID NO: 21 or SEQ ID NO: 22.

Embodiment 17. The immunoresponsive cell of any one of embodiments 1-16, wherein the chimeric receptor comprises the amino acid sequence set forth in SEQ ID NO: 21 or SEQ ID NO:

22.

Embodiment 18. The immunoresponsive cell of any one of embodiments 1-17, wherein the chimeric receptor is recombinantly expressed.

Embodiment 19. The immunoresponsive cell of any one of embodiment 1-18, wherein the chimeric receptor is expressed from a vector.

Embodiment 20. The immunoresponsive cell of any one of embodiments 1-19, wherein the immunoresponsive cell is a cell of the lymphoid lineage or a cell of the myeloid lineage.

Embodiment 21. The immunoresponsive cell of embodiment 20, wherein the cell of the lymphoid lineage is selected from T cells, B cells, Natural Killer (NK) cells, or dendritic cells.

Embodiment 22. The immunoresponsive cell of any one of embodiments 1-21, wherein the immunoresponsive cell is a NK cell or a NKG2C+CD8+ T cell.

Embodiment 23. The immunoresponsive cell of embodiment 22, wherein said cell is derived from an induced pluripotent stem cell.

Embodiment 24. The immunoresponsive cell of any one of embodiments 1-23, wherein said cell is autologous or allogeneic.

Embodiment 25. The immunoresponsive cell of any one of embodiments 1-24, wherein the immunoresponsive cell further comprises a first cytokine selected from the group consisting of an IL-15 polypeptide or a functional fragment thereof, an IL-33 polypeptide or a functional fragment thereof, an IL-18 polypeptide or a functional fragment thereof, an IL-12 polypeptide or a functional fragment thereof, an IL-2 polypeptide or a functional fragment thereof, an IL-4 polypeptide or a functional fragment thereof, an IL-7 polypeptide or a functional fragment thereof, an IL-9 polypeptide or a functional fragment thereof, and an IL-21 polypeptide or a functional fragment thereof.

Embodiment 26. The immunoresponsive cell of embodiment 25, wherein the immunoresponsive cell further comprises a second cytokine selected from the group consisting of IL-15 polypeptide or a functional fragment thereof, an IL-33 polypeptide or a functional fragment thereof, an IL-18 polypeptide or a functional fragment thereof, an IL-12 polypeptide or a functional fragment thereof, an IL-2 polypeptide or a functional fragment thereof, and an IL-4 polypeptide or a functional fragment thereof, an IL-7 polypeptide or a functional fragment thereof, an IL-9 polypeptide or a functional fragment thereof, and an IL-21 polypeptide or a functional fragment thereof.

Embodiment 26. The immunoresponsive cell of any one of embodiments 1-26, wherein the immunoresponsive cell further comprises a cytokine receptor selected from the group consisting of an IL-15 receptor polypeptide, a ST2 polypeptide, an IL-18 receptor polypeptide, an IL-12 receptor polypeptide, an IL-2 receptor polypeptide, an IL-4 receptor polypeptide, an IL-7 receptor polypeptide, an IL-9 receptor polypeptide, and an IL-21 receptor polypeptide.

Embodiment 28. The immunoresponsive cell of any one of embodiment 1-27, wherein the immunoresponsive cell further comprises an antigen-recognizing receptor that targets a second antigen.

Embodiment 29. The immunoresponsive cell of embodiment 28, wherein the second antigen-recognizing receptor is a chimeric antigen receptor (CAR), a T cell receptor (TCR), a chimeric ligand receptor, a chimeric co-stimulating receptor (CCR), or a combination thereof.

Embodiment 30. A nucleic acid encoding a chimeric receptor that binds to an antigen and that does not deliver an activation signal and a co-stimulatory signal to an immunoresponsive cell.

Embodiment 31. The nucleic acid of embodiment 30, wherein the chimeric receptor comprises an extracellular antigen-binding domain and a transmembrane domain.

Embodiment 32. The nucleic acid of embodiment 30 or 31, wherein the chimeric receptor comprises an intracellular polypeptide.

Embodiment 33. The nucleic acid of embodiment 31 or 32, wherein the transmembrane domain comprises a CD8 polypeptide, a CD28 polypeptide, a CD32 polypeptide, a CD4 polypeptide, a 4-1BB polypeptide, an OX40 polypeptide, an ICOS polypeptide, a CD84 polypeptide, a CD166 polypeptide, a CD8a polypeptide, a CD8b polypeptide, an ICAM-1 polypeptide, a CTLA-4 polypeptide, a CD27 polypeptide, a CD40 polypeptide, a NKGD2 polypeptide, a PD-1 polypeptide, a LAG-3 polypeptide, a 2B4 polypeptide, a BTLA polypeptide, a DAP12 polypeptide, a DAP10 polypeptide, a CD16 polypeptide, a DNAM1 polypeptide, or a combination thereof.

Embodiment 34. The nucleic acid of embodiment 32 or 33, wherein the intracellular polypeptide comprises a CD28 polypeptide, a CD4 polypeptide, a 4-1BB polypeptide, an OX40 polypeptide, an ICOS polypeptide, a CD84 polypeptide, a CD166 polypeptide, a CD8a polypeptide, a CD8b polypeptide, a ICAM-1 polypeptide, a CTLA-4 polypeptide, a CD27 polypeptide, a CD40 polypeptide, a NKGD2 polypeptide, a PD-1 polypeptide, a LAG-3 polypeptide, a 2B4 polypeptide, a BTLA4 polypeptide, a DAP12 polypeptide, a DAP-10 polypeptide, a CD16 polypeptide, a DNAM1 polypeptide, or a combination thereof.

Embodiment 35. The nucleic acid of any one of embodiments 32-34, wherein the intracellular polypeptide comprises a CD28 polypeptide.

Embodiment 36. The nucleic acid of any one of embodiments 32-35, wherein the intracellular polypeptide lacks a CD3 polypeptide.

Embodiment 37. The nucleic acid of any one of embodiments 30-36, further comprising a promoter that is operably linked to the antigen-recognizing receptor.

Embodiment 38. The nucleic acid of embodiment 37, wherein said promoter is endogenous or exogenous.

Embodiment 39. The nucleic acid of embodiment 38, wherein the exogenous promoter is selected from the group consisting of an elongation factor (EF)-1 promoter, a CMV promoter, a SV40 promoter, a PGK promoter, and a metallothionein promoter.

Embodiment 40. The nucleic acid of embodiment 37 or 38, wherein said promoter is an inducible promoter.

Embodiment 41. The nucleic acid of embodiment 40, wherein the inducible promoter is selected from the group consisting of a NFAT transcriptional response element (TRE) promoter, a CD69 promoter, a CD25 promoter, and an IL-2 promoter.

Embodiment 42. A vector comprising the nucleic acid of any one of embodiments 30-41.

Embodiment 43. A lipid nanoparticle comprising the nucleic acid of any one of embodiments 30-41.

Embodiment 44. A cell comprising the nucleic acid of any one of embodiments 30-41, the vector of embodiment 42, or the lipid nanoparticle of embodiment 43.

Embodiment 45. A composition comprising the nucleic acid of any one of embodiments 30-41, the vector of embodiment 42, the lipid nanoparticle of embodiment 43, or the cell of any one of embodiments 1-29 or 44.

Embodiment 46. The composition of embodiment 45, which is a pharmaceutical composition further comprising a pharmaceutically acceptable excipient.

Embodiment 47. A method of reducing tumor burden in a subject, the method comprising administering the nucleic acid of any one of embodiments 30-41, the vector of embodiment 42, the lipid nanoparticle of embodiment 42, the cell of any one of embodiments 1-29 or 44, or the composition of embodiment 45 or 46.

Embodiment 48. The method of embodiment 47, wherein the method reduces the number of tumor cells, reduces tumor size, and/or eradicates the tumor in the subject.

Embodiment 49. A method of treating a subject having a relapse of a neoplasm, the method comprising administering the nucleic acid of any one of embodiments 30-41, the vector of embodiment 42, the lipid nanoparticle of embodiment 43, the cell of any one of embodiments 1-29 or 44, or the composition of embodiment 45 or 46.

Embodiment 50. A method of treating and/or preventing a neoplasm in a subject, the method comprising administering the nucleic acid of any one of embodiments 30-41, the vector of embodiment 42, the lipid nanoparticle of embodiment 43, the cell of any one of embodiments 1-29 or 44, or the composition of embodiment 45 or 46.

Embodiment 51. The method of any one of embodiments 47-50, wherein the neoplasm or tumor is cancer.

Embodiment 52. The method of any one of embodiments 47-51, wherein the neoplasm or tumor is selected from the group consisting of blood cancers and solid tumors.

Embodiment 53. The method of embodiment 52, wherein the blood cancer is multiple myeloma, myeloid disorder, leukemia, or lymphoma.

Embodiment 54. The method of embodiment 53, wherein the leukemia is acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, or B cell prolymphocytic leukemia.

Embodiment 55. The method of embodiment 54, wherein the leukemia is acute myeloid leukemia (AML).

Embodiment 56. The method of embodiment 55, wherein the lymphoma is Hodgkin's lymphoma, non-Hodgkin's lymphoma, B-cell non-Hodgkin's lymphoma, or T-cell non-Hodgkin's lymphoma.

Embodiment 57. The method of embodiment 52, wherein the solid tumor is selected from the group consisting of renal cell carcinoma, non-small-cell lung cancer, lung adenocarcinoma, lung squamous cell carcinoma, lung neuroendocrine carcinoma, small-cell lung cancer, pancreatic cancer, breast cancer, astrocytoma, glioblastoma, laryngeal/pharyngeal carcinoma, EBV-associated nasopharyngeal carcinoma, and ovarian carcinoma.

Embodiment 58. The method of any one of embodiments 47-57, wherein the subject is a human.

Embodiment 59. A method for producing a cell, the method comprising introducing into a cell the nucleic acid of any one of embodiments 30-41, the vector of embodiment 42, or the lipid nanoparticle of embodiment 43.

Embodiment 60. A kit comprising the nucleic acid of any one of embodiments 30-41, the vector of embodiment 42, the lipid nanoparticle of embodiment 43, the cell of any one of embodiments 1-29 or 44, or the composition of embodiment 45 or 46.

Embodiment 61. The kit of embodiment 60, wherein the kit further comprises written instructions for treating and/or preventing a neoplasm, a pathogen infection, and/or an infectious disease.

Embodiment 62. The nucleic acid of any one of embodiments 30-41, the vector of embodiment 42, the lipid nanoparticle of embodiment 42, the cell of any one of embodiments 1-29 or 44, or the composition of embodiment 45 or 46 for use in reducing tumor burden in a subject.

Embodiment 63. The nucleic acid, the vector, the lipid nanoparticle, the cell, or the composition for use of embodiment 62, wherein the use reduces the number of tumor cells, reduces tumor size, and/or eradicates the tumor in the subject.

Embodiment 64. The nucleic acid of any one of embodiments 30-41, the vector of embodiment 42, the lipid nanoparticle of embodiment 42, the cell of any one of embodiments 1-29 or 44, or the composition of embodiment 45 or 46 for use in treating a subject having a relapse of a neoplasm.

Embodiment 65. The nucleic acid of any one of embodiments 30-41, the vector of embodiment 42, the lipid nanoparticle of embodiment 42, the cell of any one of embodiments 1-29 or 44, or the composition of embodiment 45 or 46 for use in treating and/or preventing a neoplasm in a subject.

Embodiment 66. The nucleic acid, the vector, the lipid nanoparticle, the cell, or the composition for use of embodiment 64 or 65, wherein the neoplasm or tumor is cancer.

Embodiment 67. The nucleic acid, the vector, the lipid nanoparticle, the cell, or the composition for use of any one of embodiments 62-66, wherein the neoplasm or tumor is selected from the group consisting of blood cancers and solid tumors.

Embodiment 68. The nucleic acid, the vector, the lipid nanoparticle, the cell, or the composition for use of embodiment 67, wherein the blood cancer is multiple myeloma, myeloid disorder, leukemia, or lymphoma.

Embodiment 69. The nucleic acid, the vector, the lipid nanoparticle, the cell, or the composition for use of embodiment 68, wherein the leukemia is acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, or B cell prolymphocytic leukemia.

Embodiment 70. The nucleic acid, the vector, the lipid nanoparticle, the cell, or the composition for use of embodiment 69, wherein the leukemia is acute myeloid leukemia (AML).

Embodiment 71. The nucleic acid, the vector, the lipid nanoparticle, the cell, or the composition for use of embodiment 68, wherein the lymphoma is Hodgkin's lymphoma, non-Hodgkin's lymphoma, B-cell non-Hodgkin's lymphoma, or T-cell non-Hodgkin's lymphoma.

Embodiment 72. The nucleic acid, the vector, the lipid nanoparticle, the cell, or the composition for use of embodiment 67, wherein the solid tumor is selected from the group consisting of renal cell carcinoma, non-small-cell lung cancer, lung adenocarcinoma, lung squamous cell carcinoma, lung neuroendocrine carcinoma, small-cell lung cancer, pancreatic cancer, breast cancer, astrocytoma, glioblastoma, laryngeal/pharyngeal carcinoma, EBV-associated nasopharyngeal carcinoma, and ovarian carcinoma.

Embodiment 73. The nucleic acid, the vector, the lipid nanoparticle, the cell, or the composition for use of any one of embodiments 62-72, wherein the subject is a human.

### EXAMPLES

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides disclosed herein, and, as such, may be considered in making and practicing the presently disclosed subject matter. Particularly useful techniques for particular embodiments will be discussed in the sections that follow.

The following examples are put forth so as to provide those of ordinary skill in the art with a complete disclosure and description of how to make and use the antibodies, multi-specific antibodies, compositions comprising thereof, screening, and therapeutic methods of the presently disclosed subject matter, and are not intended to limit the scope of what the inventors regard as their presently disclosed subject matter. It is understood that various other embodiments may be practiced, given the general description provided above.

### Example 1—Preclinical Evaluation of Chimeric Antigen Receptor-Modified Natural Killer Cells Targeting Membrane-Proximal CD33 in Acute Myelogenous Leukemia Summary

Augmenting the cytotoxic potential of natural killer (NK) cells through the expression of a chimeric antigen receptor (CAR) offers avenues to overcome many of the limitations of CAR-modified T cells, notably T cell-associated toxicities and difficulties with off-the-shelf allogeneic use. For example, the presently disclosed subject matter reports the generation of membrane-proximal CD33-targeted CAR-modified NK cells produced by retroviral transduction of peripheral blood-derived primary NK cells. These CAR-modified NK cells displayed stable CAR expression and phenotype, excellent proliferation, and augmented short- and long-term cytotoxicity, serial killing, and NK cell activation against CD33-positive AML cell lines compared to non-transduced or mock-CAR-modified NK cells in vitro. Their short- and long-term cytotoxicity, serial killing, and proliferation were improved in vitro by the addition of cytokine transgenes (IL-15, IL-33) to the CAR construct. While excessive NK cell proliferation by CAR-IL15 was associated with significant systemic toxicity in vivo, this was attenuated by the addition of IL33 (CAR-IL-33-IL-15). In fact, mice treated with CAR-IL-33-IL-15 had the best tumor control and overall survival. In conclusion, a novel immunotherapy approach was developed using engineered NK cells, which are easy to produce and exhibit promising efficacy.

### Introduction

Acute myelogenous leukemia (AML) is a rapidly progressing neoplastic disorder characterized by the accumulation of malignant myeloid precursor cells in the bone marrow. AML has an aggressive clinical course in both adults and children, and relapsed/refractory acute myelogenous leukemia (AML) has a very poor prognosis (Gamis et al., Pediatr Blood Cancer. 2013; 60 (6): 964-971). Despite the addition of novel agents to the intensive chemotherapeutic backbone and consolidative allogeneic stem cell transplantation therapy, durable remission remains rare (Rasche et al., Cancers (Basel). 2021; 13 (10)). Among the novel therapeutic strategies being developed for AML are chimeric antigen receptor (CAR)-modified T cells, several types of which have been clinically introduced for other advanced hematologic malignancies (Maude et al., N Engl J Med. 2018; 378 (5): 439-448). CAR modification involves forced expression of a tumor-specific Ig fragment (e.g., a single-chain variable fragment) joined with an activating intracellular component derived from T cell receptors, thereby linking antibody target recognition of lineage-specific surface markers to the potent effector mechanisms of T cells (Dotti et al., Immunol Rev. 2014; 257 (1): 107-126). Despite their clinical efficacy against CD19-expressing malignancies and BCMA-expressing multiple myeloma, the use of CAR-modified T cells can be limited, in certain instances, by HLA restriction, high failure rate in production from heavily pretreated patients, highly costly and time-consuming collection and manufacturing processes, and poor durability of response in treated patients (Sterner et al., Blood Cancer Journal. 2021; 11 (4): 69). To circumvent these limitations, off-the-shelf CAR T cell therapy approaches have been developed; however, these present significant concerns surrounding tolerability, the need for severe immunosuppression, the safety of gene editing, and the risk for graft-versus-host disease (GvHD) (Depil et al., Nat Rev Drug Discov. 2020; 19 (3): 185-199).

An attractive alternative to T cells for allogeneic effector cell CAR modification and use in cancer therapy are natural killer (NK) cells. NK cells are immune effector cells that play a pivotal role in first-line defense against virally infected or tumor-transformed cells (Lodoen et al., Curr Opin Immunol. 2006; 18 (4): 391-398). NK cell function is not HLA-restricted and, in fact, may be enhanced in the setting of donor-recipient human leukocyte antigen (HLA) mismatch (Grossenbacher et al., Immunotherapy. 2017; 9 (6): 487-497). As engineered NK cells retain their full array of native receptors, they can exert antitumor cytotoxicity through mechanisms other than that dictated by CAR specificity, especially in the setting of AML, which lacks expression of HLA class I (Boudreau et al., Curr Opin Immunol. 2018; 50:102-111). This in principle can reduce the risk of relapse attributed to tumoral loss of CAR-targeted antigen, a phenomenon known to occur in CAR T cell therapy (Rezvani et al., Mol Ther. 2017; 25 (8): 1769-1781). Umbilical cord-derived CAR-modified NK cells have recently proven efficacious with minimal toxicities: no cytokine release syndrome (CRS) or GvHD has been observed in patients with CD19-expressing malignancies (Liu et al., N Engl J Med. 2020; 382 (6): 545-553). This success is being extended to other sources of NK cells and other malignancies (Pang et al., Cancers (Basel). 2022; 14 (17)). A possible source is peripheral blood, but these have a relatively short lifespan of approximately 14 days. However, this could be leveraged to minimize on-target off-tumor toxicities that have been observed with longer-lasting T cells, especially in the setting of myeloid disease (Wang et al., Mol Ther. 2015; 23 (1): 184-191). Developing CAR-modified immune cells for AML requires a tumor-associated antigen for targeting. The most promising is CD33, a sialoadhesin consisting of membrane-distal immunoglobulin variable (IgV) and membrane-proximal immunoglobulin constant (IgC2) domains, given its near-ubiquitous expression on AML cells, particularly leukemic stem cells (LSCs), and its association with inferior clinical outcomes (Willier et al., Blood. 2021; 137 (8): 1037-1049; Pollard et al., Blood. 2012; 119 (16): 3705-3711; Pollard et al., J Clin Oncol. 2016; 34 (7): 747-755). Preclinical studies have reported that CD33-targeted CAR T and NK cells show potent activity (Haiying et al., J Immunother Cancer. 2021; 9 (9): e003149; Albinger et al., Blood Cancer J. 2022; 12 (4): 61). Among these, 3P14HLh28ζ, a CD33-directed CD28/CD3ζ-based CAR T cell derived from a high-affinity binder obtained through membrane-proximal fragment immunization was recently developed. Compared to the current best-in-class CAR T cells derived from gemtuzumab and lintuzumab, which target distal CD33 epitopes with high affinity or membrane-proximal epitopes with low affinity, 3P14HLh28ζ showed enhanced in vitro functionality as well as superior tumor control and increased overall survival in xenograft models with low antigen density and high tumor burden.

### Methods

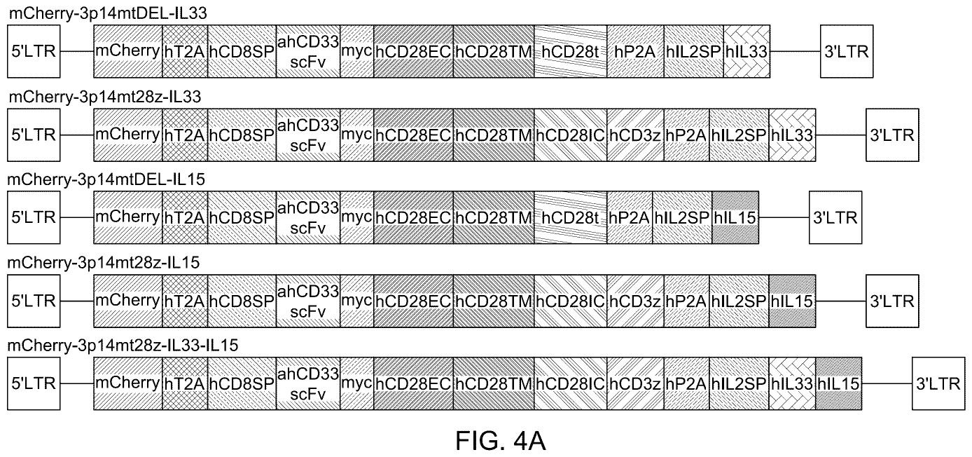

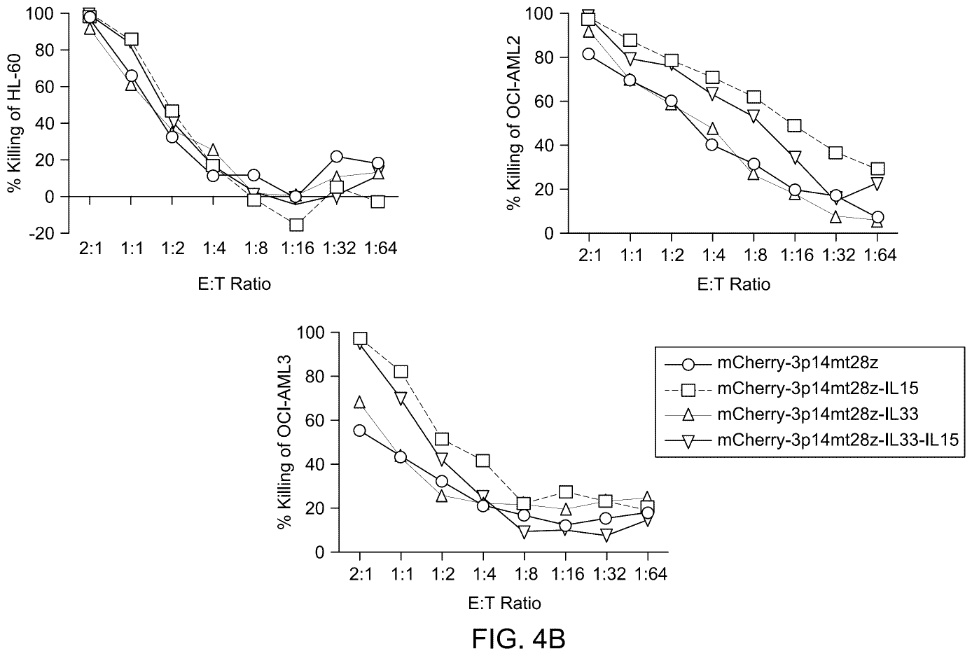

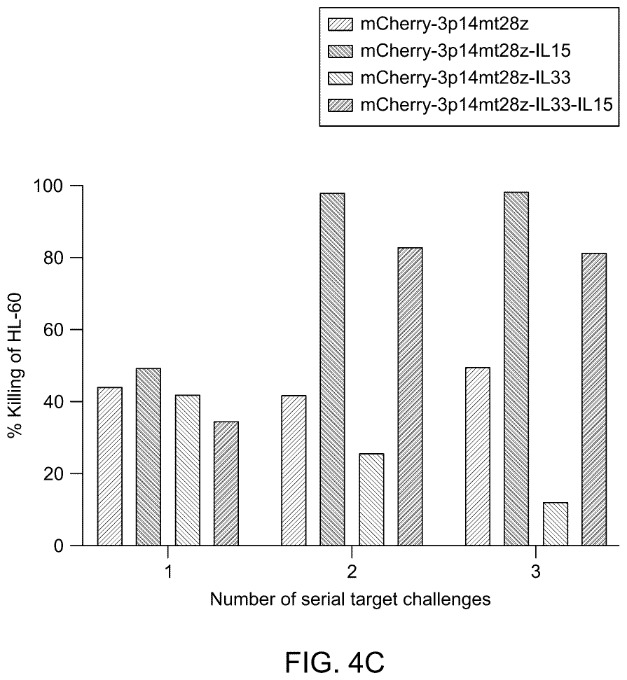

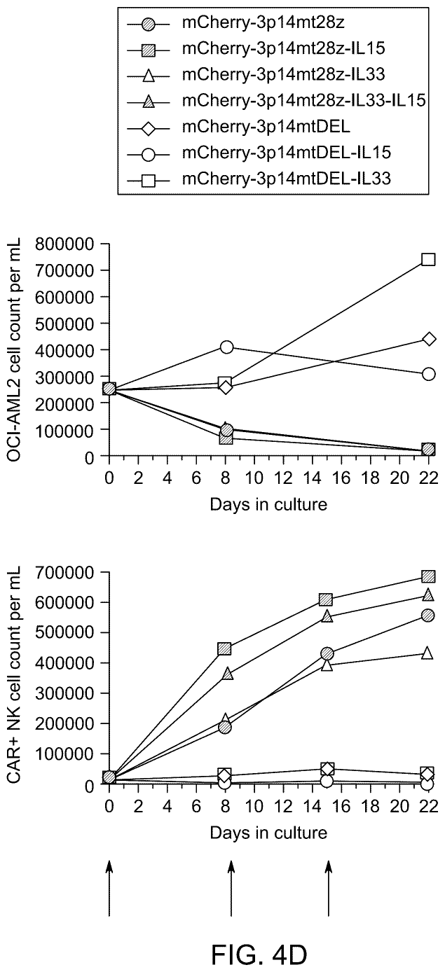

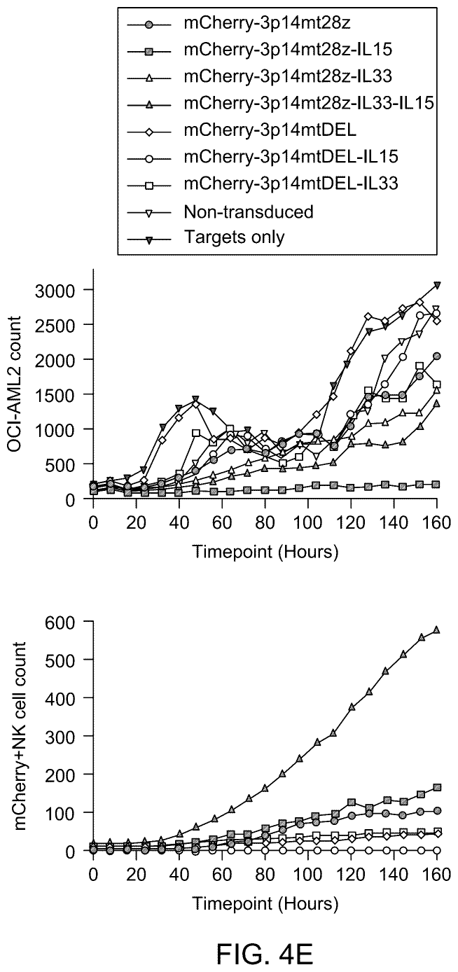

Generation of CAR constructs. 3P14HLh28ζ was previously described in International Patent Application No. PCT/US2020/000000). 3P14HLDEL was generated by deletion of the cytosolic domain of CD28 and CD3ζ. mCherry sequence was linked preceding the CAR construct via an hT2A element. Genes encoding the cytokines IL-33 and/or IL-15 were linked by a hP2A element followed by hIL2SP after the CAR construct. In the IL-33-IL-15-coexpressing construct, the genes were fused by a G4S linker (FIGS. 1A and 4A).

Generation of retroviral constructs. Constructs were cloned into the SFG gammaretroviral vector with human signaling domains. Retroviral producer cell lines were generated using CaPO4 (Promega) according to the manufacturer's instructions to transiently transfect gpg29 fibroblasts (H29) with retroviral constructs encoding the CAR. Supernatant from H29 cells was used to transduce 293Glv9 cells to produce stable retroviral producer cell lines.

NK cell isolation, retroviral transduction, and expansion. Peripheral blood mononuclear cells (PBMCs) were isolated from healthy donor PB or leukopacks using density gradients (Ficoll, GE Healthcare). Following red blood cell lysis using ACK (ammonium-chloride-potassium) lysing buffer (Lonza), human CD56+NK cells were isolated from PBMCs using an NK isolation kit (Miltenyi Biotec, Inc., San Diego, CA, USA). Isolated NK cells were then activated with 500 IU/mL of IL-2 (PeproTech) and irradiated (100 Gy) artificial antigen-presenting cells (aAPCs) expressing membrane-bound IL-21 (C9.mbIL21 cells; courtesy of Dean Lee, Nationwide Children's Hospital, OH) at a feeder: NK cell ratio of 2:1 in a GREX plate. After 6 days, activated NK cells were transduced with retroviral supernatant collected from 293Glv9 packaging cells on RetroNectin-coated plates on 2 consecutive days (Takara Clontech). Four days later, NK cells were stimulated again with irradiated C9.mbIL21 and transferred to a GREX plate and expanded for 9-18 additional days. RPMI-1640 medium supplemented with 10% heat-inactivated FCS, 1% penicillin/streptomycin, 5% human serum, and 1% L-glutamine was changed every 6 days and supplemented with 500 IU/mL of IL-2 every 3 days. Transduction efficiency was determined by flow cytometric analysis. All experiments were normalized for CAR+ viable cells.

Flow cytometry. Flow cytometric analyses were performed using 14-color LSR Fortessa (BD Biosciences) and 14-color Attune NxT (Thermo Fisher Scientific) instruments. Flow cytometry was used to determine transduction efficiency of transduced cells following staining with Myc-tag (9B11, Cell Signaling), mCherry, and F(ab′) 2 Fragment Goat Anti-Human IgG (Jackson ImmunoResearch). DAPI (0.5 mg/mL, Sigma Aldrich) or LIVE/DEAD™ Fixable Aqua Dead Cell Stain Kit (405 nm, Invitrogen) staining were used to exclude dead cells in all experiments. The following antihuman antibodies were used for flow cytometry: anti-CD3 (UCHT1), anti-CD56 (B159), anti-CD33 (WM53), anti-KIR3DL1 (DX9) purchased from BD Biosciences; anti-NKG2A (REA110) purchased from Miltenyi Biotec; anti-NKG2C (134591), anti-KIR2DL1 (143211) purchased from R&D Systems; anti-NKG2C (134591), anti-KIR2DL1 (143211) purchased from R&D Systems; anti-TIM3 (F38-2E2), anti-TIGIT (A15153G), anti-PD1 (EH12.2H7), anti-PD-L1 (29E.2A3), and anti-CD16 (3G8) purchased from BioLegend; anti-KIR2DL2/L3/S2 (GL183) purchased from Beckman Coulter. Data were analyzed using FlowJo software (10.7.1).

Cell lines. 293Glv9-packaging cells were maintained in high-glucose Dulbecco's Modified Eagle Medium supplemented with 10% heat-inactivated fetal bovine serum, nonessential amino acids (Atlanta Biological Flowery Branch), 2 mM L-glutamine (Invitrogen), and 1% penicillin/streptomycin (Invitrogen). OCI-AML2 and OCI-AML3 human acute myeloid leukemia cells and HL60 human acute myeloid leukemia cells were modified to express GFP-firefly luciferase to detect tumor in vitro and in vivo by luminescence. Tumor lines were sorted by fluorescence-activated cell sorting (FACS) based on high expression of GFP. All tumor cell lines were maintained in RPMI-1640 medium supplemented with 10% heat-inactivated FCS and 1% penicillin/streptomycin. All cells were maintained at 37° C., 5% CO₂. Cell lines were routinely tested for potential mycoplasma contamination.

CD33-knockout AML cell lines. HL60 and OCI-AML2 cells were transfected by electrotransfer of modified Cas9 mRNA (tri-link) and gRNA using an AgilePulse MAX system (BTX Harvard Apparatus). 2×10⁵ cells were mixed with 5 μg Cas9 mRNA (Thermo Fisher Scientific) and 5 μg of CD33 gRNA guide (TrueGuide synthetic gRNA, Thermo Fisher Scientific). The target DNA sequence of the sgRNA guide was: 5′-GGCCGGGTTCTAGAGTGCCA-3′ (SEQ ID NO: 138). Following 3 pulses of electroporation (600V, 600V, 100V), cells were diluted in complete RPMI medium and incubated for 72 h, after which knockout efficiency was assessed by surface expression of CD33 via flow cytometry. Cells in the lowest 10% of CD33 expression were expanded to generate the knockout line.

Cytotoxicity assays. The cytolytic capacity of CAR-modified human NK cells was assessed using luciferase-killing assays. CAR NK cells were co-cultured with 1×10⁴ target cells: HL60-gfpLuc+ or OCI-AML2-gfpLuc+ or OCI-AML3-gfpLuc+ tumor cells, at various effector-to-target ratios in triplicate or quadruplicate in white-walled 96-well plates (Corning) in a total volume of 200 μL of cell media. Target cells alone were plated at the same cell density to determine the maximal luciferase expression as a reference. After 24 h, 75 ng of D-luciferin (Gold Biotechnology) dissolved in 50 μL of PBS was added to each well and the luminescence of each sample was detected in a MicroBeta2 Microplate Counter (Perkin Elmer) and quantified using the MicroBeta2 Windows Workstation Version 2.3.0.12 software (Perkin Elmer). Percent lysis was determined as (1-(sample signal/max signal))×100. For serial killing assays, 100 μL supernatant was removed every 24-48 h and replaced with 1×10⁴ target cells in 100 μL of media supplemented with 200 IU/mL IL-2. For long-term killing assays, CAR T cells and tumor cells were co-cultured at various effector-to-target ratios in duplicate and imaged over 138-168 h using the Sartorius IncuCyte S3. Tumor cell killing was measured as total green count over time and CAR-modified NK cell expansion as total red count over time.

Proliferation assays. CAR NK cells were co-cultured with OCI-AML2-gfpLuc⁺ tumor cells at 1:5 NK: tumor cell ratio. After 7 days, flow cytometry was used to detect tumor (gfp+) and CAR+NK cells (mCherry+). CAR NK cells were re-stimulated with fresh tumor cells at the same ratio and proliferation measured every 7 days for 14-35 days after initial stimulation.

Functional assays. CD107a mobilization and IFN-γ production were assessed to determine NK cell activation. Frozen PBMC samples were thawed and incubated overnight in complete RPMI media with 200 U/mL IL-2. PBMCs (5× 10⁵ cells per well) or NK cells (1×10⁵ cells per well) were incubated in 96-well V bottom plates with target cells at a 1:1 ratio in the presence of anti-CD107a antibody (BD Biosciences, catalog #563869). After 2 h co-culture, 2.5 μg/mL of brefeldin A (MP Biomedicals) and 1:3000 BD GolgiStop (containing 0.26% monensin) was added. After another 4 h, cells were washed, fixed/permeabilized, and stained with anti-IFNγ antibody (BD Biosciences, catalog #557995).

In vitro and in vivo IL-15 cytokine analysis. To measure in vitro NK cell cytokine production, CAR NK cells were co-cultured at a 1:1 ratio for 24 h with CD33+ or CD33-tumor cells in a 96-well round bottom plate for 24 h. Cytokine content of supernatant was measured on a Luminex IS100 instrument. For in vivo cytokine analysis, peripheral blood was drawn from NCG mice 17 days after tumor injection, and serum was analyzed for cytokines on a Luminex FlexMap3D system using Luminex xPONENT 4.2 software. Human IL-15, GM-CSF, IFN-γ, IL-2, and TNF-α were assayed using the Human Cytokine/Chemokine/Growth Factor Panel A kit (MilliporeSigma).

Animal models. All experiments were performed in accordance with an Institutional Animal Care and Use Committee (IACUC)-approved protocol. For xenogeneic studies, NOD-Prkdc^{em26Cd52}Il2rg^{em26CD22}/NjuCrl, coisogenic immunodeficient (NCG) mice were purchased from Charles River and subsequently housed under specific-pathogen-free (SPF) conditions. For all experiments, 6- to 8-week-old mice were used.

In vivo experiments. NCG mice were inoculated via tail vein with 5×10⁵ OCI-AML2-gfpLuc+ tumor cells on day 0. On day 3, mice were blindly randomized into treatment cohorts and treated with 1×10⁷ NK cells via tail vein. Tumor burden was measured weekly via bioluminescence imaging using the Xenogen IVIS Imaging System (Xenogen) with Living Image software (PerkinElmer). Wherever indicated, mice received 0.5 μg (2.5 units) recombinant human IL-15 (Miltenyi Biotech) intraperitoneally on the day of NK cell infusion and twice per week thereafter. Mice were euthanized when they had hind limb paralysis or reached a moribund state. The Genetically Modified Animal Phenotyping Core at MSK performed and interpreted necropsies, histopathology, immunohistochemistry, hematology, and serum chemistry.

Necropsy and histopathology. Mice were euthanized by exposure to CO₂ in accordance with IACUC guidelines. Following gross examination all organs were fixed in 10% neutral buffered formalin, followed by decalcification of bone in a formic acid solution (Surgipath Decalcifier I, Leica Biosystems). Tissues were then processed in ethanol and xylene and embedded in paraffin in a Leica ASP6025 tissue processor. Paraffin blocks were sectioned at 5 μm, stained with hematoxylin and eosin (H&E), and examined by a board-certified veterinary pathologist. Tissues were processed and examined from all major organs.

Immunohistochemistry. Immunohistochemistry for mCherry and GFP was performed on paraffin sections of selected tissues using a Leica Bond RX automated stainer. After heat-induced epitope retrieval in pH 6.0 buffer (for mCherry only), anti-mCherry mouse monoclonal antibody clone 1C51 (Abcam ab 125096) and anti-GFP rabbit polyclonal antibody (Invitrogen A6455) were applied at 1:5000 and 1:500, respectively, followed by detection using a polymer reagent kit according to the manufacturer's instructions (DS9800, Novocastra Bond Polymer Refine Detection, Leica Biosystems). The chromogen indicating positive immunoreactivity was 3,3 diaminobenzidine tetrachloride (DAB) and sections were counterstained with hematoxylin.

Hematology. Blood was collected into tubes containing EDTA. Automated analysis was performed on an IDEXX Procyte DX hematology analyzer and the following parameters were determined: white blood cell count, red blood cell count, hemoglobin concentration, hematocrit, mean corpuscular volume, mean corpuscular hemoglobin, mean corpuscular hemoglobin concentration, red blood cell distribution width standard deviation and coefficient of variance, reticulocyte relative and absolute counts, platelet count, platelet distribution width, mean platelet volume, and relative and absolute counts of neutrophils, lymphocytes, monocytes, eosinophils, and basophils. A blood smear was prepared, stained with Wright-Giemsa, and evaluated by a medical technologist for morphology of red blood cells, white blood cells and platelets, and white blood cell differential count.

Serum chemistry. Blood was collected into tubes containing a serum separator, which were centrifuged to isolate serum. Serum chemistry was performed on a Beckman Coulter AU680 analyzer and the concentration of the following analytes was determined: alkaline phosphatase, alanine aminotransferase, aspartate aminotransferase, creatine kinase, gamma-glutamyl transpeptidase, albumin, total protein, globulin, total bilirubin, blood urea nitrogen, creatinine, cholesterol, triglycerides, glucose, calcium, phosphorus, chloride, potassium, and sodium. Na/K ratio and albumin/globulin ratio were calculated.

Statistical analysis. All statistical analyses were performed using Prism 8 software (GraphPad). Data points represent biological replicates and are shown as mean±standard error of the mean (SEM). Statistical significance was determined by unpaired t-test, one-way ANOVA, or two-way ANOVA as indicated. The log-rank (Mantel-Cox) test was used to determine statistical significance for overall survival in mouse experiments. Significance was indicated with *, P<0.05; **, P<0.01; ***, P<0.001; and ****, P<0.0001.

### Results

Peripheral Blood-Derived NK Cells can be Stably Transduced with a Retroviral Vector to Express Anti-CD33 Chimeric Antigen Receptors

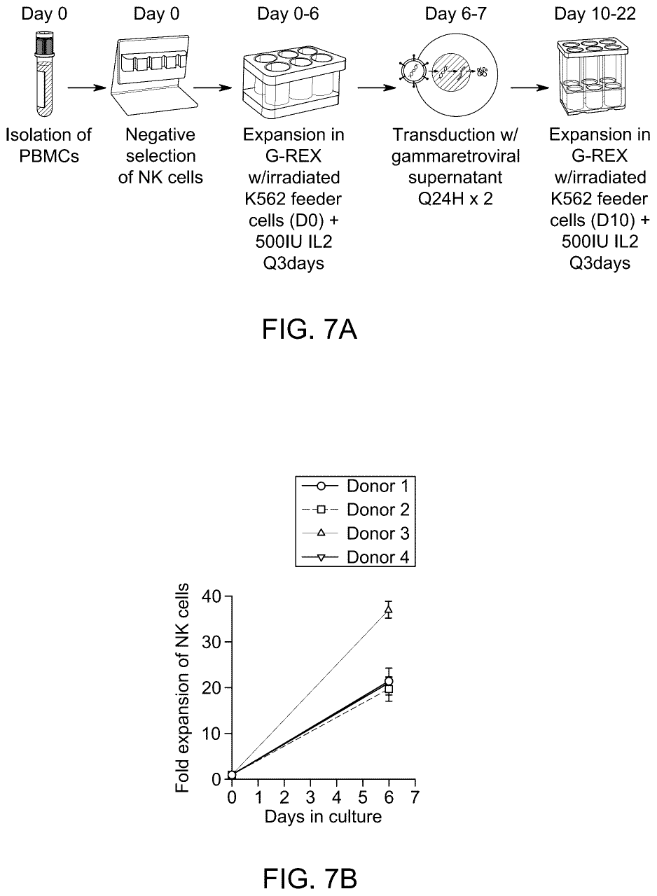

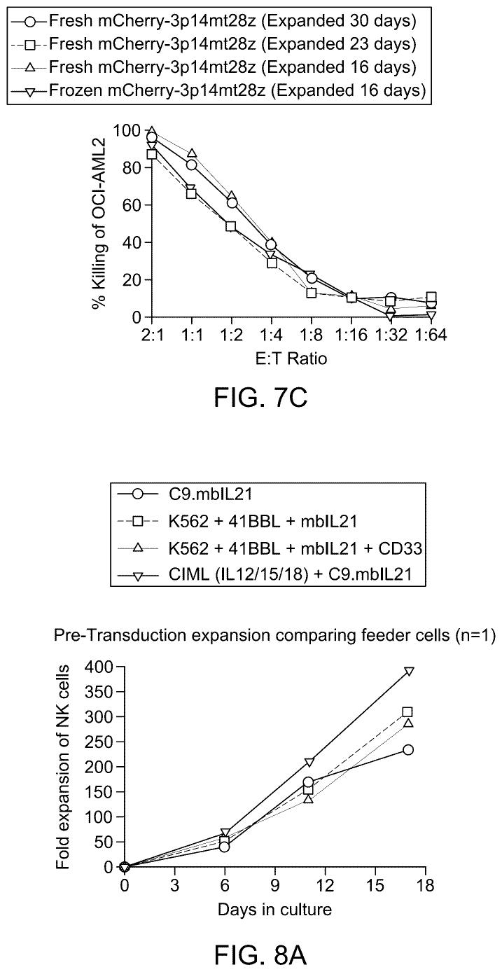

To evaluate NK cell expansion and transduction, NK cells were isolated from the peripheral blood of healthy donors and expanded them using the K562-based feeder cells C9.mbIL21 and IL-2 (see Materials and Methods, FIGS. 7A and 7B). Expanded cells were transduced on days 6 and 7 with retroviral vectors (FIG. 1A) and cultured for an additional 9-18 days depending on downstream application (see Materials and Methods, FIG. 7A). mCherry-3p14mt28ζ, a second-generation CAR, was modified to remove the intracellular signaling domains (CD28 and CD32) to serve as a control, mCherry-3p14mtDEL, since the signaling domains are essential for linking the antigen-cell binding event to a cascade of intracellular molecular events (FIG. 1A). NK cell viability on day 4 after transduction was ≥95% in all cases. The median transduction efficiency was 71.9% for mCherry alone, 43.6% for mCherry-3p14mtDEL, and 56.9% for mCherry-3p14mt28ζ (FIG. 1B). All constructs were evaluated in 3-4 healthy donors with varying HLA/killer cell immunoglobulin-like receptor (KIR) types and NK cell education status (Table 2).

TABLE 2
HLA typing, inhibitory KIRs present,
and NK cell education for donors 1-4

| | Donor 1 | Donor 2 | Donor 3 | Donor 4 |
|---|---|---|---|---|
| HLA Typing | Bw6 | Bw4 | Bw4 | Bw6 |
| | Bw6 | Bw6 | Bw6 | Bw6 |
| | C1 | C1 | C1 | C1 |
| | C1 | C2 | C2 | C2 |
| Inhibitory KIRs | KIR2DL3 | KIR3DL1 | KIR3DL1 | KIR3DL1 |
| Present | | KIR2DL3 | KIR2DL3 | KIR2DL3 |
| | | KIR2DL1 | KIR2DL1 | KIR2DL1 |
| NK Cell | Low | High | High | Intermediate |
| Education | | | | |

Note:
KIRs in bold contribute to NK cell education

A minority of in vivo experiments were completed using cryopreserved NK cells, which demonstrated comparable cytotoxicity to fresh NK cells in vitro (FIG. 7C).

The stability of CAR expression over time was studied by culturing CAR-transduced NK cells from 4 donors for 5 weeks. CAR expression remained stable over this interval, as determined by flow cytometry every 10-14 days. There was no significant difference in CAR expression when NK cells were transduced with different constructs (FIGS. 1B-D). Given the relatively high level of co-expression of human IgG, myc tag, and mCherry (FIGS. 1C and 1D), mCherry was used as a surrogate marker of CAR expression for all subsequent assays.

Peripheral Blood-Derived NK Cells Transduced with a Retroviral Vector to Express Anti-CD33 Chimeric Antigen Receptors Expand Robustly without a Significant Change in Phenotype

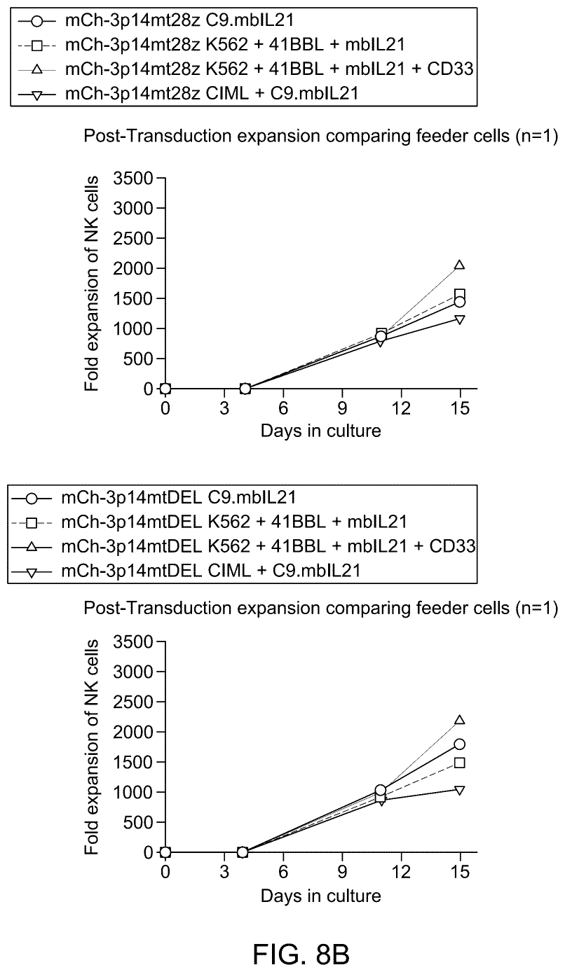

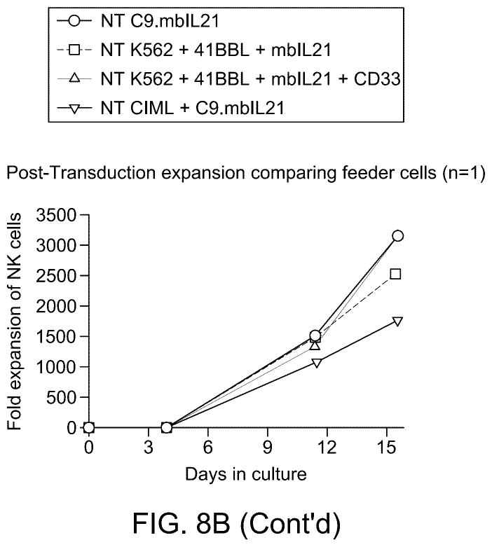

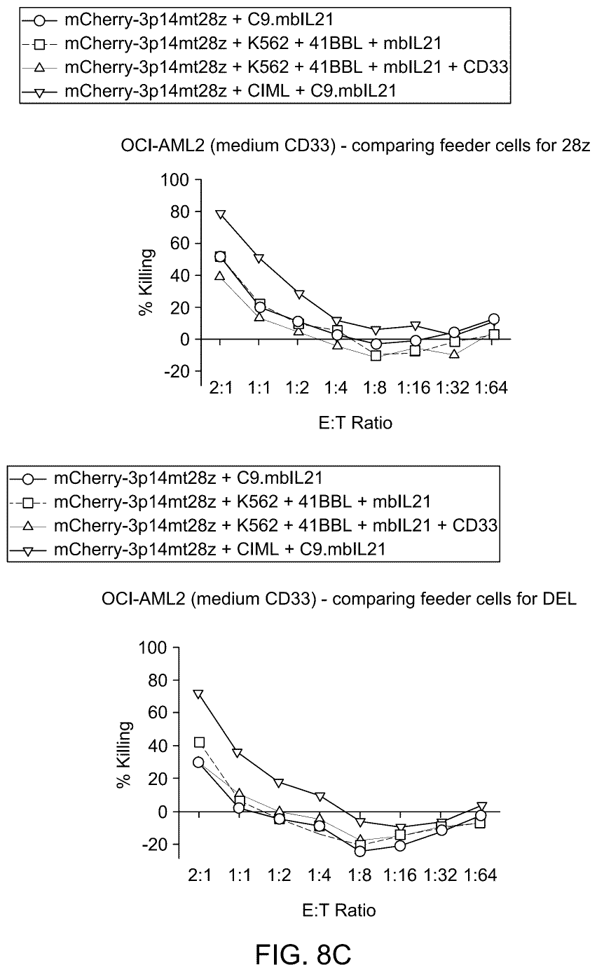

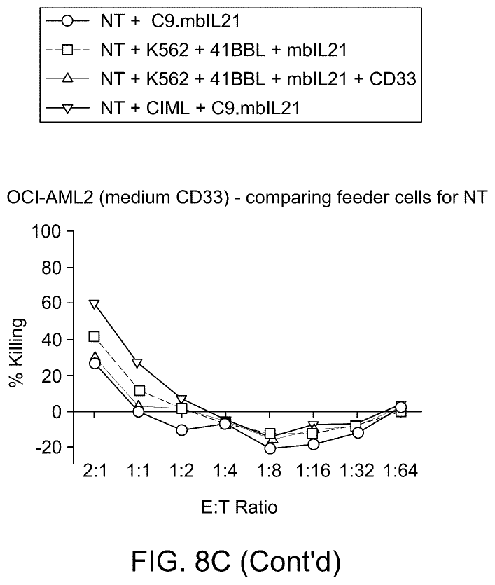

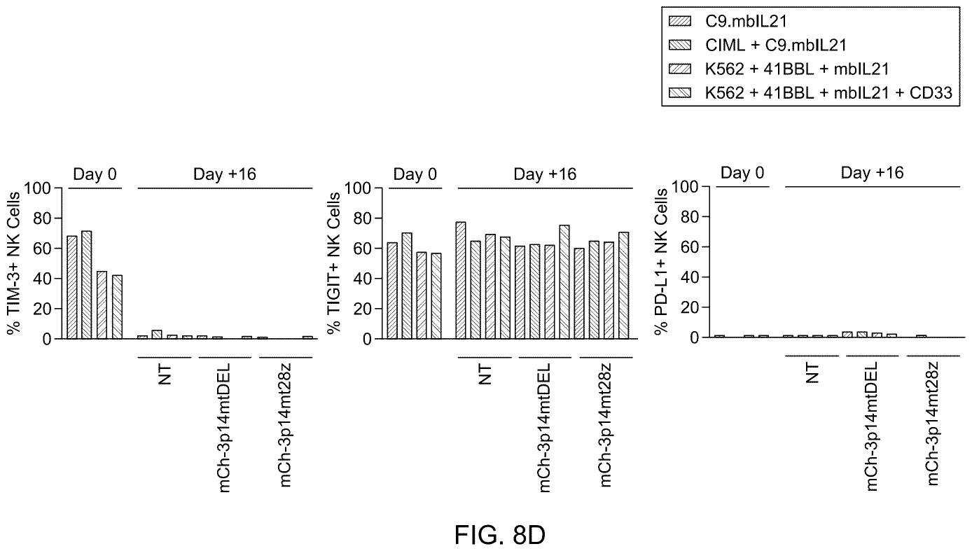

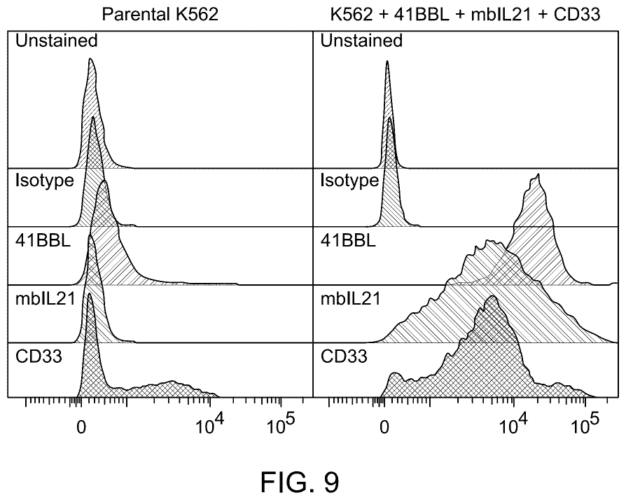

To address the well-known challenge of NK cell expansion, an NK cell expansion protocol was developed using G-REX plates and K562-derived feeder cells, C9.mbIL21, which express CD64, CD86, 41BBL, membrane-bound IL21 (mbIL21), and truncated CD19 (FIG. 7A). It was also analyzed the expansion in the presence of C9.mbIL21 feeder cells, another line of K562-based feeder cells expressing 41BBL and mbIL21 with or without CD33, or cytokine-induced memory-like (CIML) NK cells activated overnight with IL-12/15/18 (FIGS. 8A-8D and 9). Pre-transduction expansion was best using CIML-NK cells (FIG. 8A); however, post-transduction expansion was best using K562s+41BBL+mbIL21+CD33 (FIG. 8B). Expanded NK cells' 24-h cytotoxicity against OCI-AML2-gfpLuc+ was best following expansion using CIML-NK cells (FIG. 8C). Expression of exhaustion markers (TIM3, TIGIT, and PD-L1) after 16 days of post-transduction expansion was relatively comparable (FIG. 8D) among feeder cell types. Given prior validation and clinical use of C9.mbIL21, all subsequent experiments were completed with these feeder cells.

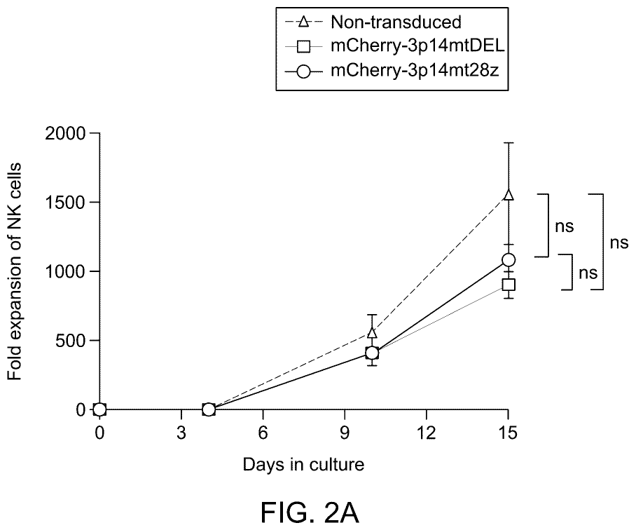

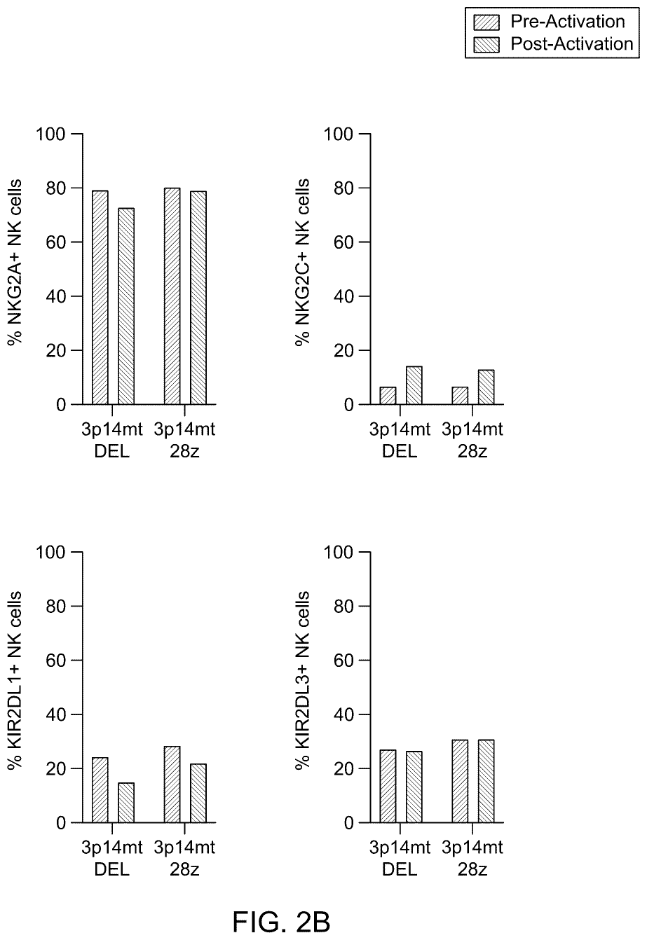

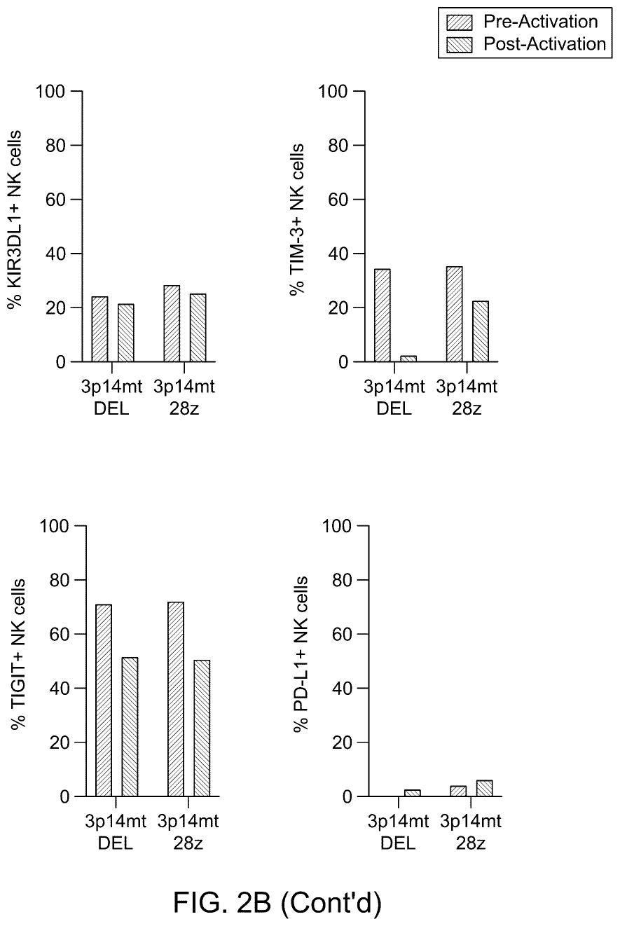

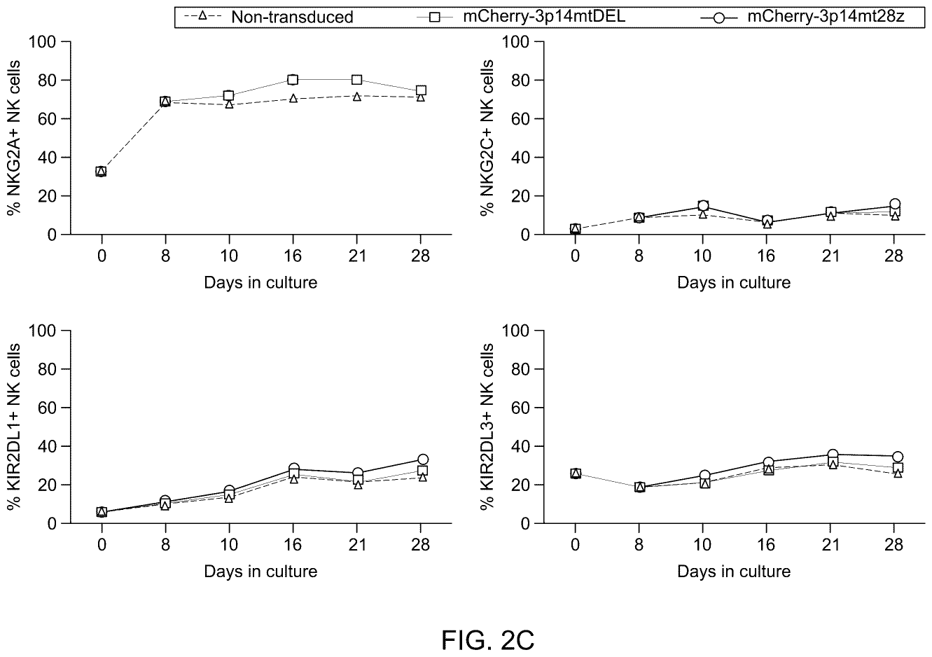

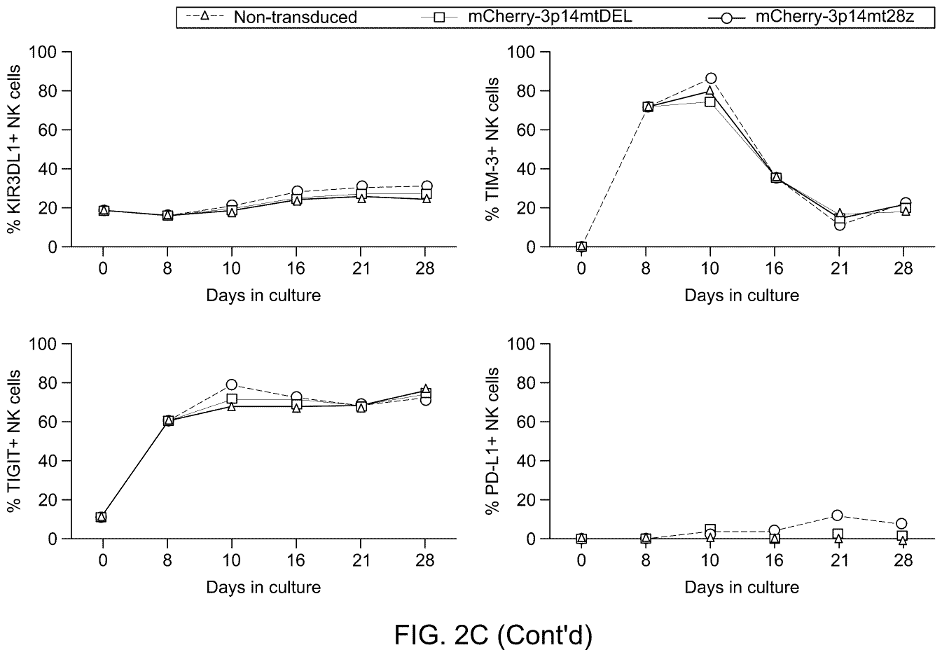

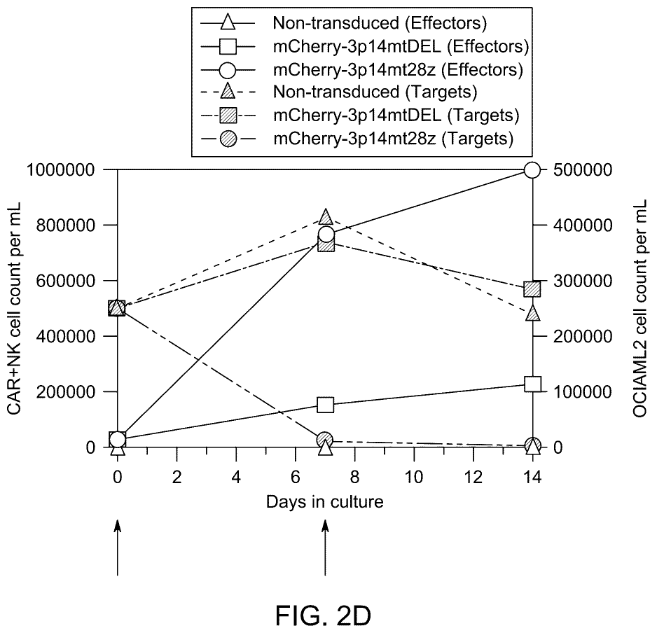

After 15 days, median NK cell expansion was 1600-fold for non-transduced NK cells and 991-fold for transduced NK cells (FIG. 2A). Expression of the CAR construct did not affect expression of key NK cell receptors or immune checkpoints (NKG2A, NKG2C, KIR2DL1, KIR2DL2, KIR3DL1, TIM3, TIGIT) (FIG. 2B). To evaluate whether deriving NK cells from peripheral blood rather than umbilical cord blood affects the stability of CAR+NK cell phenotype, it was examined the expression of these markers over 28 days of expansion in a representative donor with highly educated NK cells. While expansion resulted in an increase in NKG2A, NKG2C, KIR2DL1, and TIGIT expression, decrease in TIM3 expression, and overall stable KIR2DL3, KIR3DL1, and PD-L1 expression, these changes were similar regardless of whether NK cells were transduced or the construct used (FIG. 2C). This demonstrates that functional (mCherry-3p14mt28z) or non-functional, i.e., lacking intracellular signaling domains (e.g., CD28 and CD3) (mCherry-3p14mtDEL) CAR modification of mature PB-derived NK cells does not significantly skew the cells' phenotype or result in exhaustion upon activation or expansion. In a proliferation assay against OCI-AML2-gfpLuc+ cells (added every 7 days), mCherry-3p14mt28ζ reduced NK cell proliferation, while mCherry-3p14mtDEL and non-transduced NK cells did not (FIG. 2D).

Transduction with Anti-CD33-28ζ Chimeric Antigen Receptors Enhances Peripheral Blood-Derived NK Cell Activation and Cytotoxicity Against CD33+ Tumor Targets In Vitro

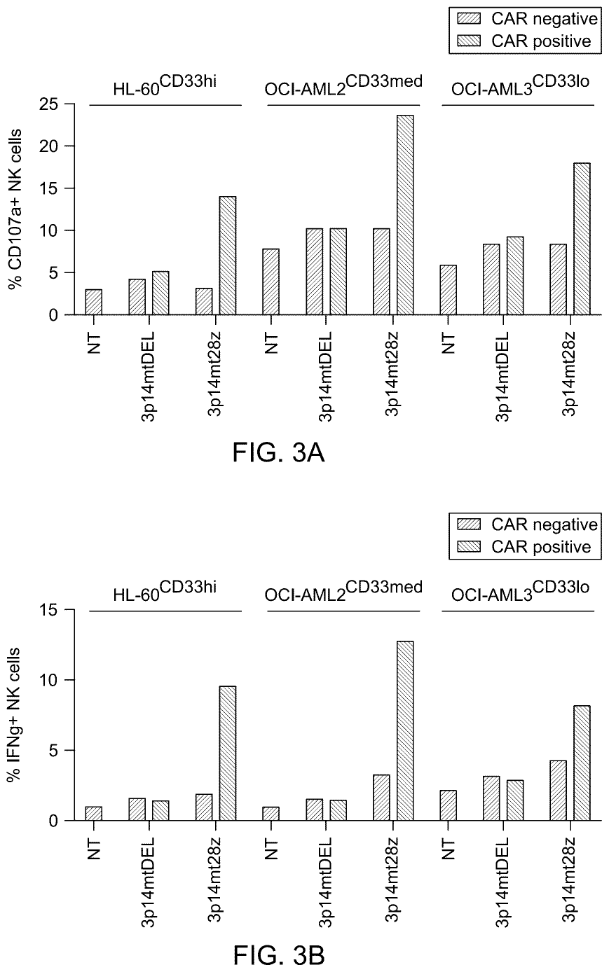

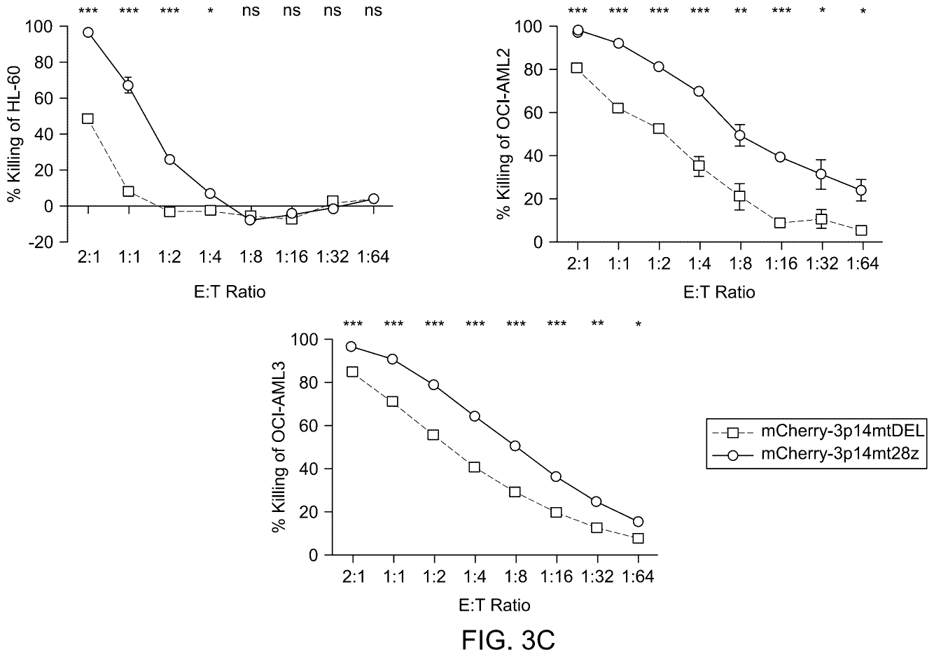

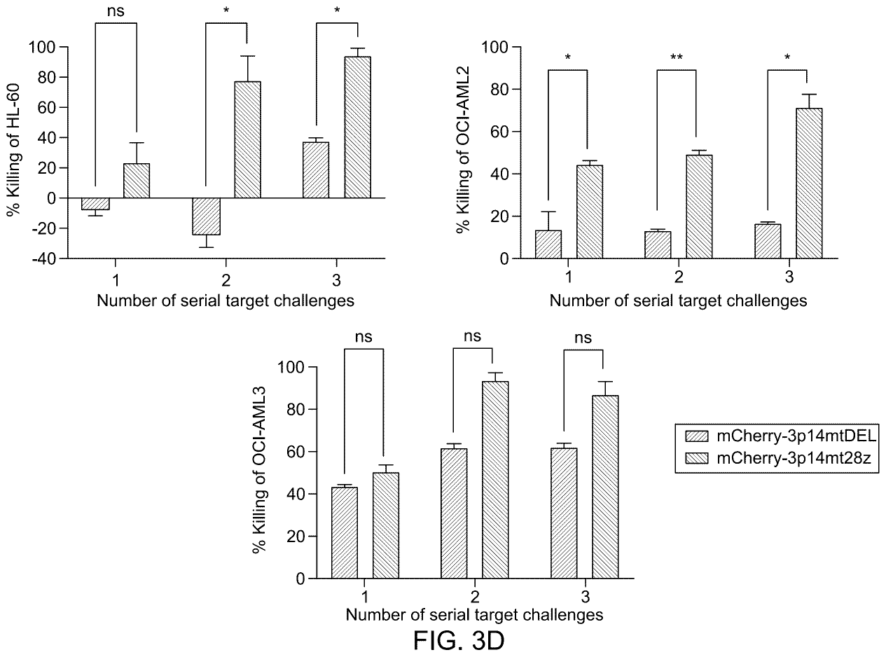

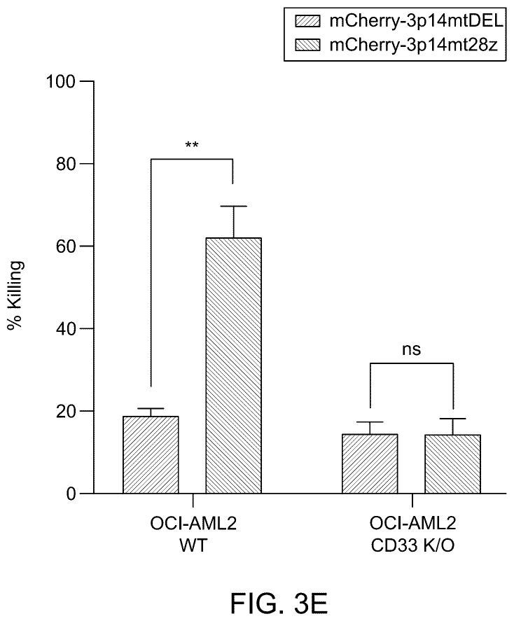

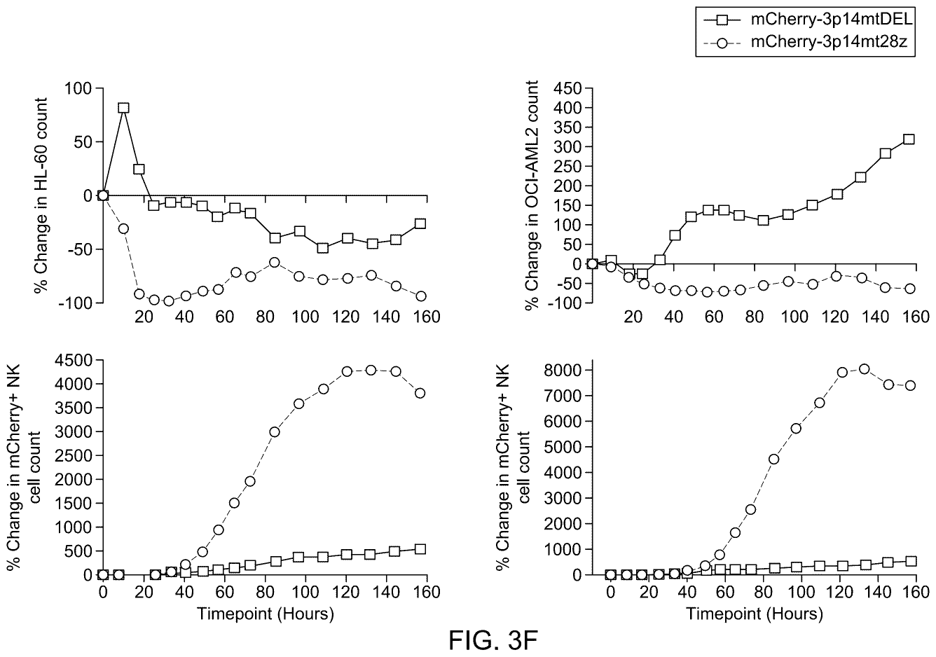

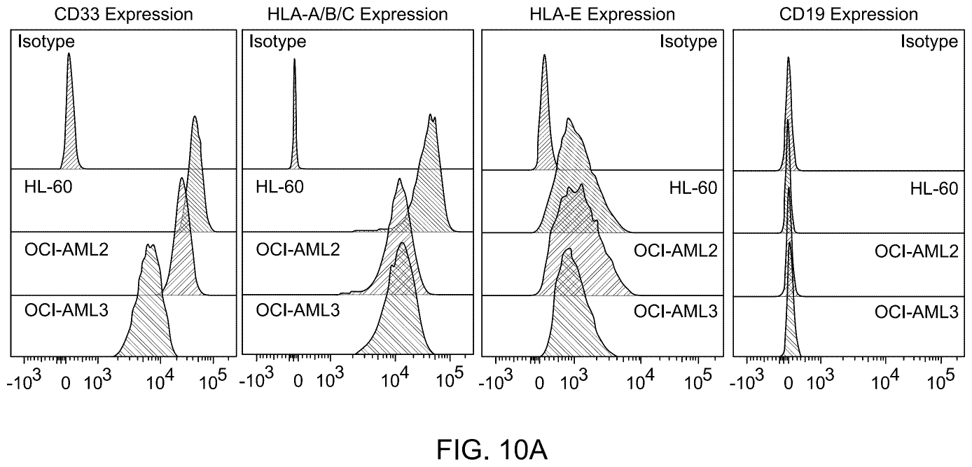

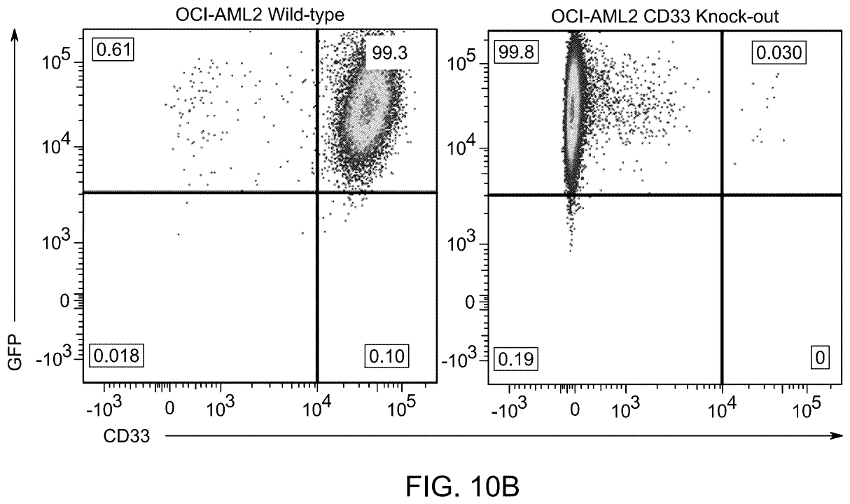

To demonstrate that enhanced activation against CD33 targets by CAR-modified NK cells is derived from the mCherry-3p14mt28ζ-expressing fraction of the product, CD107a degranulation (FIG. 3A) and interferon (IFN)-γ response (FIG. 3B) to HL60-gfpLuc+, OCI-AML2-gfpLuc+, and OCI-AML3-gfpLuc+ cell lines were measured. These cell lines express relatively high, medium, and low levels of CD33, respectively, and have different HLA profiles (FIG. 10A and Table 3).

TABLE 3
HLA typing for AML cell lines.

| | | | OCI-AML2 | | OCI-AML3- | |
|---|---|---|---|---|---|---|
| | HL-60-GFPLuc+ | | GFPLuc+ | | GFPLuc+ | |
| HLA-A Allele 1 | 01:01 | | 02:01 | | 23:01 | |
| HLA-A Allele 2 | 01:01 | | 02:01 | | 02:01 | |
| HLA-B Allele 1 | 57:01 | (Bw4 BI80) | 18:01 | (Bw6) | 44:02 | (Bw4 N77 T80) |
| HLA-B Allele 2 | 57:01 | (Bw4 BI80) | 15:01 | (Bw6) | 53:01 | (Bw4 I80) |
| HLA-C Allele 1 | 06:02 | (C2) | 03:03 | (C1) | 04:01 | (C2) |
| HLA-C Allele 2 | 06:02 | (C2) | 07:01 | (C1) | 05:01 | (C2) |

Next, it was observed that CAR+ (mCherry+) NK cells produced more CD107a and interferon-γ in response to CD33+ targets than CAR-(mCherry-) cells or 3p14mtDEL-transduced or non-transduced NK cell controls.

Next, it was evaluated whether engineering peripheral blood-derived NK cells to express mCherry-3p14mt28ζ effectively enhanced their cytotoxicity against CD33-expressing tumors compared to mCherry-3p14mtDEL. Across all E:T ratios, mCherry-3p14mt28ζ-transduced NK cells exerted superior killing of all 3 AML cell lines compared to mCherry-3p14mtDEL (FIG. 3C). This superior cytotoxicity was maintained after serial target challenges every 24 h (FIG. 3D).

To determine the antigen-negative cytotoxic capacity of transduced NK cells, OCI-AML2-gfpLuc+ CD33 knockout cells were established (FIG. 10B). mCherry-3p14mt28ζ-transduced NK cells were equally as efficient as mCherry-3p14mtDEL-transduced NK cells in killing OCI-AML2-gfpLuc+CD33 knockout cells (FIG. 3E), indicating that the enhanced killing of CD33+ targets by CAR-modified NK cells is mediated by the CAR and not related to a nonspecific enhancement in NK cytotoxicity.

To evaluate long-term cytotoxicity, it was also tested whether expression of mCherry-3p14mt28ζ enhanced peripheral blood-derived NK cells' cytotoxicity against CD33-expressing tumors in a long-term (160-h) co-culture assay. mCherry-3p14mt28ζ-transduced NK cells reduced numbers of HL60-gfpLuc+ and OCI-AML2-gfpLuc+ cells, while mCherry-3p14mtDEL-transduced NK cells did not (FIG. 3F).

Peripheral Blood-Derived antiCD33 Chimeric Antigen Receptor-Modified NK Cells' In Vitro Cytotoxicity is Further Enhanced by the Addition of IL-15

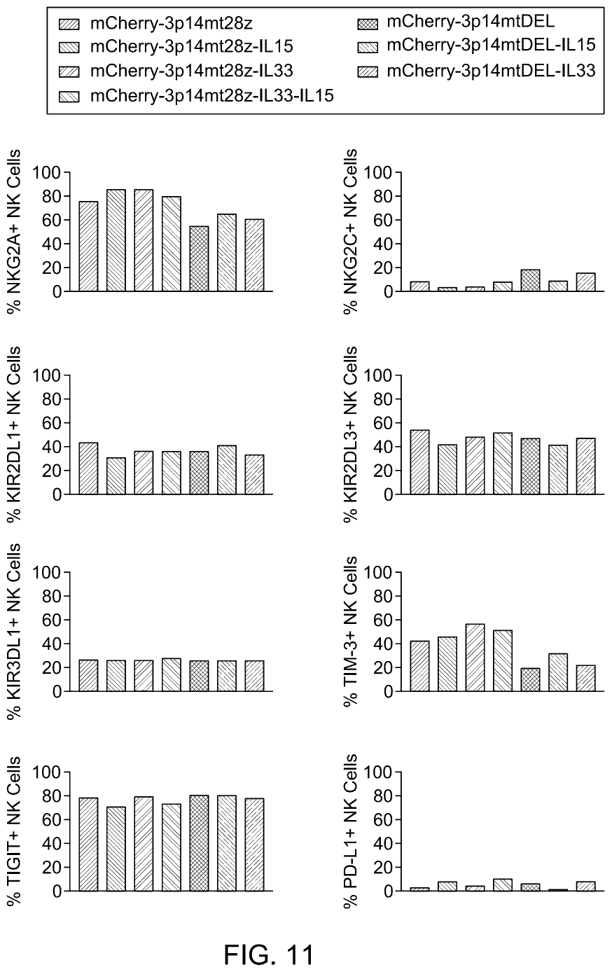

To further enhance NK cell proliferation, survival, and cytotoxicity, IL-33, IL-15, or both were added to the mCherry-3p14mt282 and mCherry-3p14mtDEL CAR constructs (FIG. 4A). IL-33 and IL-15 expression was comparable between constructs as assessed by western blot and ELISA. The addition of IL-15 to the construct, either alone or fused to IL-33, enhanced 24-h killing of HL60-gfpLuc+ (limited to lower E:T ratios), OCI-AML2-gfpLuc+, and OCI-AML3-gfpLuc+ (FIG. 4B) and serial killing of HL60-gfpLuc+ (FIG. 4C) compared to the CAR-only construct. In a 22-day assay of CAR-positive NK cells' inhibition of OCI-AML2 cells' proliferation in which the latter were added every 7 days, NK cells expressing all 28z constructs controlled tumor, but NK cells transduced with IL-15-containing constructs proliferated most (FIG. 4D). In a 160-h long-term killing assay in which OCI-AML2 cells were added once at hour 0, NK cells expressing the CAR and IL-15-only best controlled tumor cell proliferation, but those expressing the CAR and IL-15-IL-33 proliferated most (FIG. 4E). Addition of IL-33 to the construct did not affect CAR-mediated NK cell killing or NK cell proliferation in either assay. After 15 days of expansion, there was no significant difference in phenotype of the various CAR+NK cells (FIG. 11).

Peripheral Blood-Derived NK Cells Transduced with CD33-Directed Chimeric Antigen Receptors Exert Antitumor Efficacy in Xenograft Models of AML

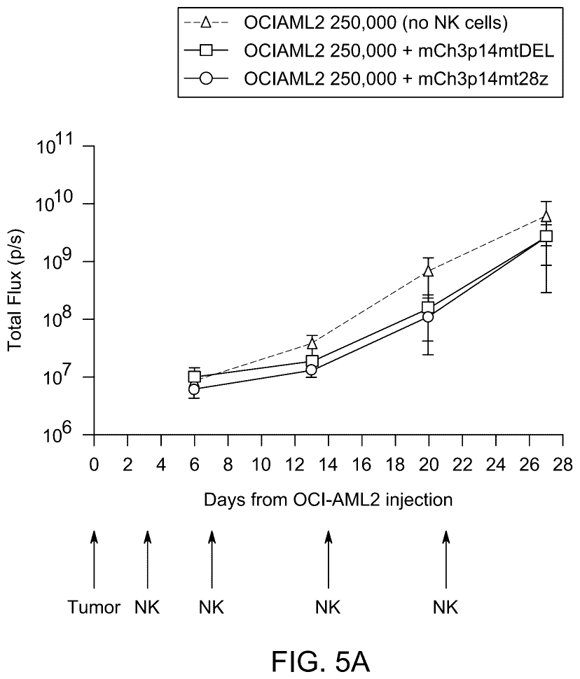

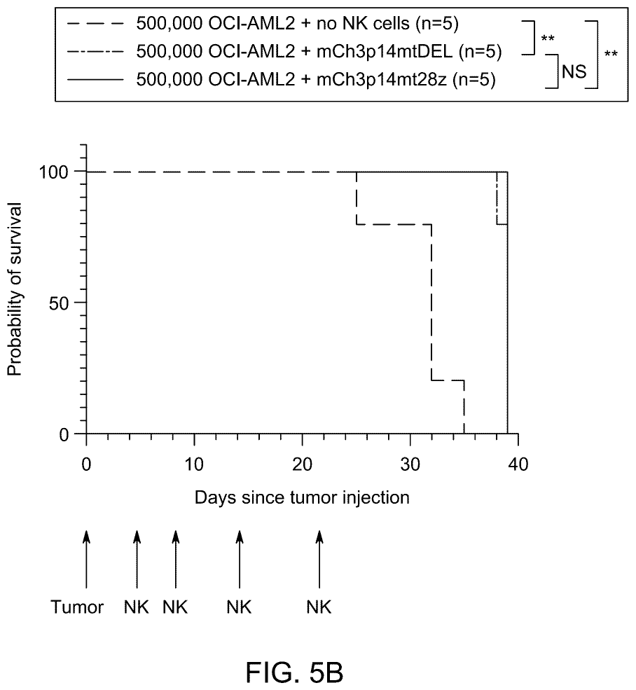

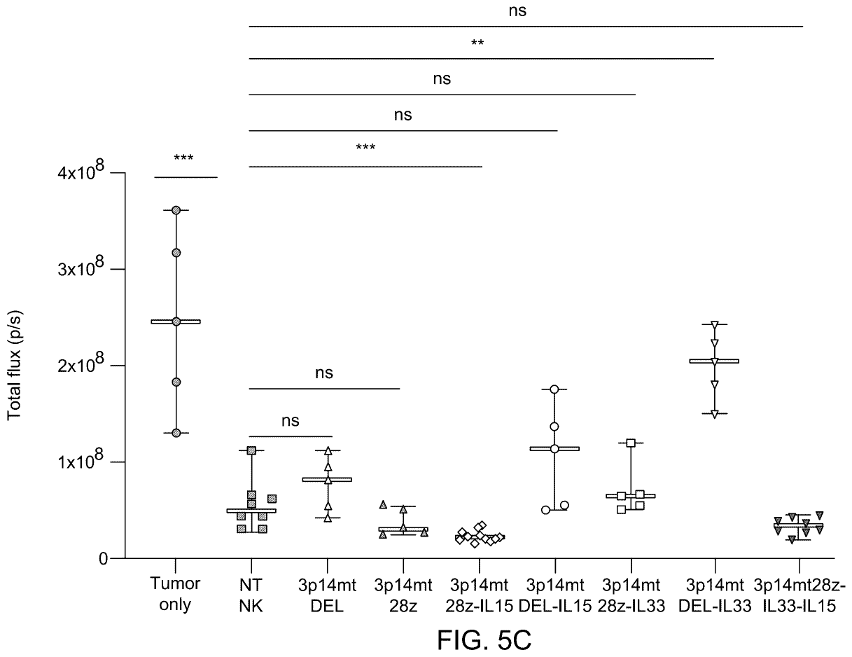

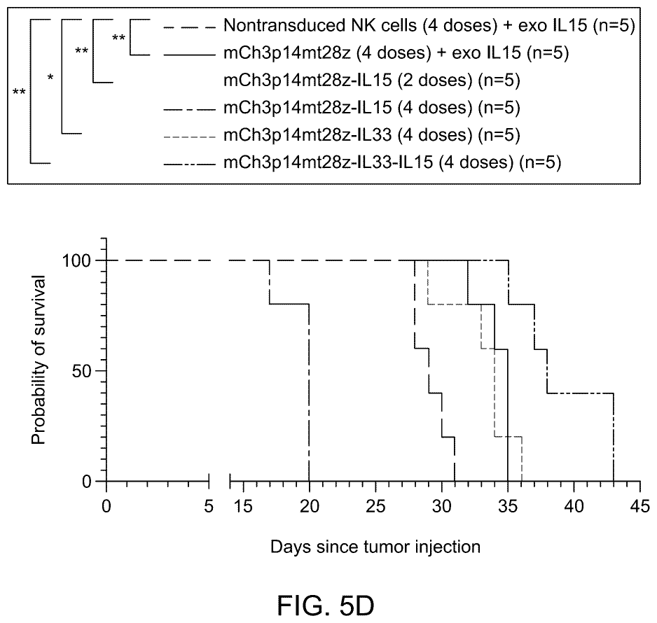

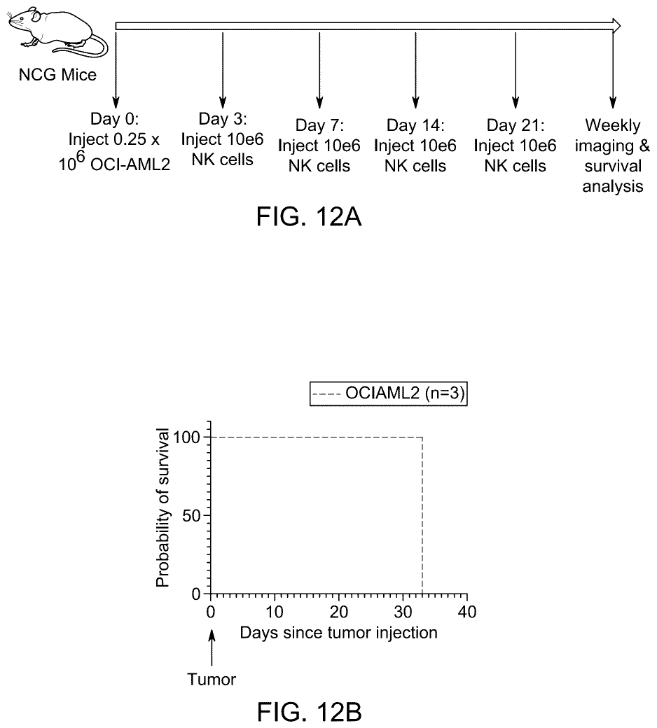

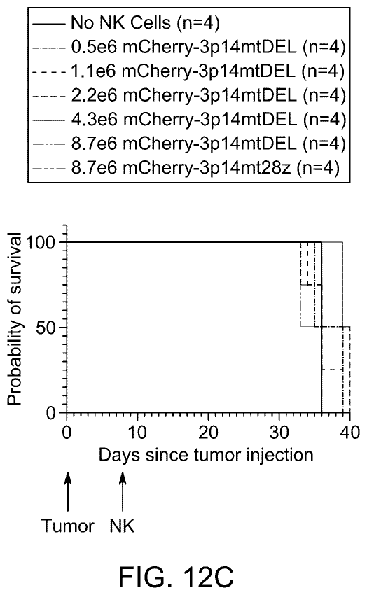

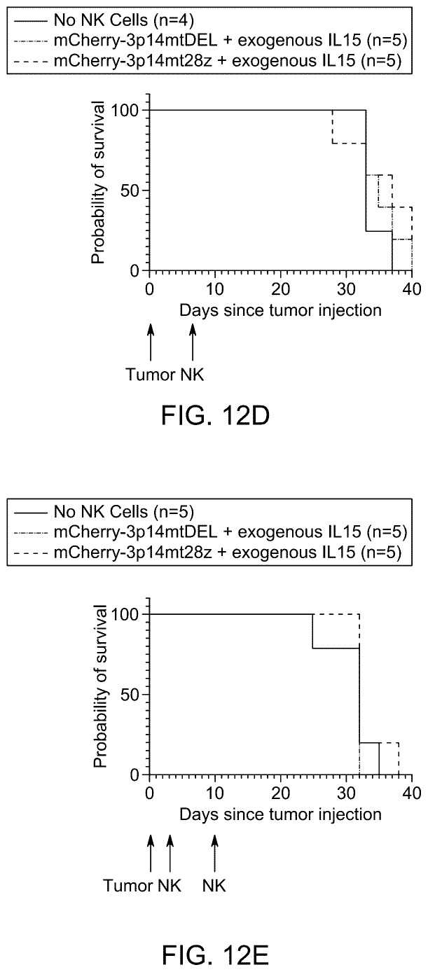

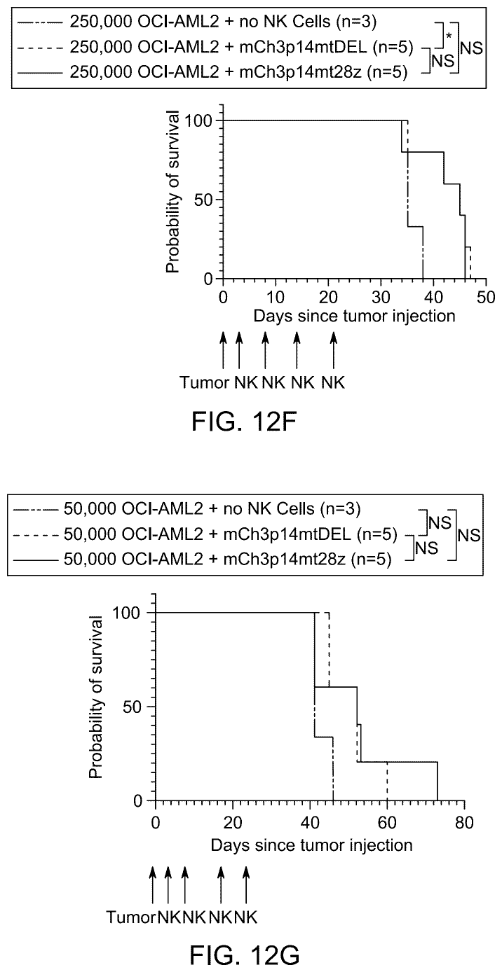

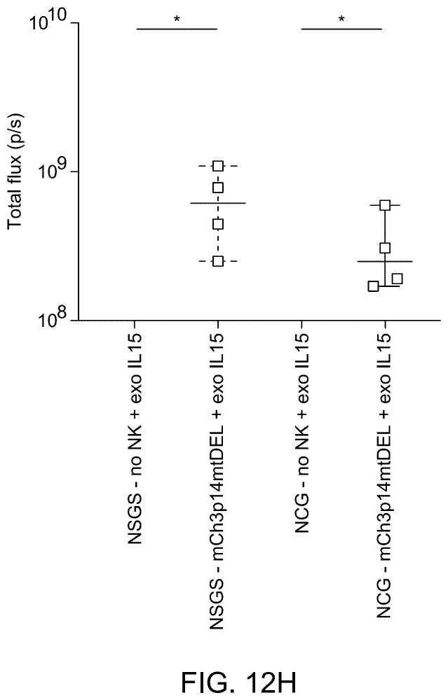

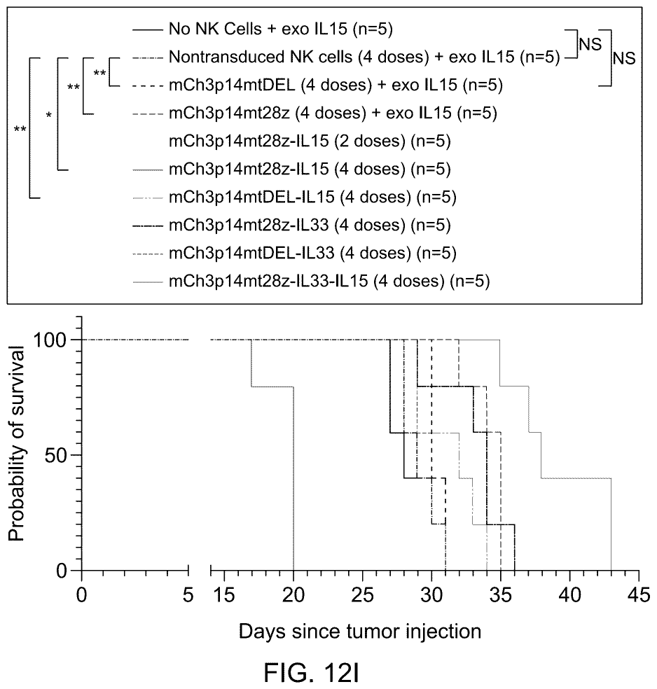

To assess in vivo efficacy, OCI-AML2-gfpLuc+ AML cells were engrafted into NCG mice. the dosages of tumor cells, NK cells, and exogenous cytokines were titrated (FIG. 12A). Using a single tumor dose of 0.5×10⁶ cells injected via tail vein at day 0, mouse survival without any treatment was 33 days (FIG. 12B). Next, NK cell dose was evaluated under the assumption that mCherry-3p14mtDEL modified NK cells should have inherent (non-CAR-mediated) anti-AML cytotoxicity in vivo. Because varying doses of NK cells (0.5×10⁶ cells to 8.7×10⁶ cells) given as a single injection yielded similar tumor and survival (FIG. 12C), which was not improved by exogenous human IL-15 (0.5 μg/mouse intraperitoneal twice weekly) (FIG. 12D), the number of doses of 10×10⁶NK cells was increased first to 2 on days 3 and 10 after tumor engraftment (FIG. 12E), then to 4 on days 3, 7, 14, and 21 with exogenous IL-15 twice weekly (for non-cytokine-secreting CAR constructs) (FIG. 5A). mCherry-3p14mt28ζ and mCherry-3p14mtDEL both decreased tumor burden and extended survival in this OCI-AML2 xenograft mouse tumor model, though survival did not differ between the two constructs (FIGS. 5A and 5B). This equivalence was also observed with higher and lower tumor engraftment doses (0.25×10⁶ and 0.05×10⁶) (FIGS. 12F and 12G) and in NSG mice (FIG. 12H).

In vivo tumor control was best maintained by NK cells expressing mCherry-3p14mt28z-IL-15 and mCherry-3p14mt282-IL33-IL-15 (FIGS. 5C and 5D); however, early death was observed in mice treated with 2 or 4 doses of NK cells expressing mCherry-3p14mt282-IL-15. Survival was prolonged in mice treated with NK cells expressing mCherry-3p14mt282-IL33-IL15 (FIGS. 5D and 12I). Together, these data suggest that engineering peripheral blood-derived NK cells to express a CD-33 directed CAR and IL-15 provides tumor control but causes early toxicity in vivo and that IL33-IL15 enhances antitumor activity while preventing IL-15-associated toxicity in vivo.

### Peripheral Blood-Derived CD33-Directed CAR NK Cells Expressing IL-15 Display Significant Toxicity In Vivo, but the Addition of IL33 Transgene Attenuates the Phenotype In Vivo

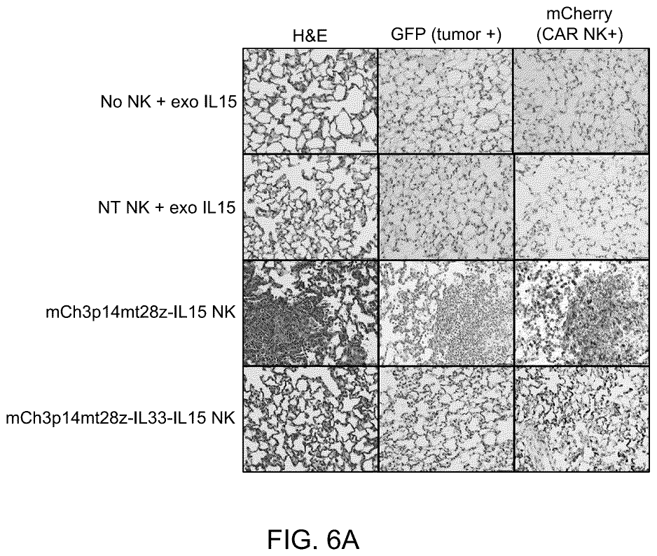

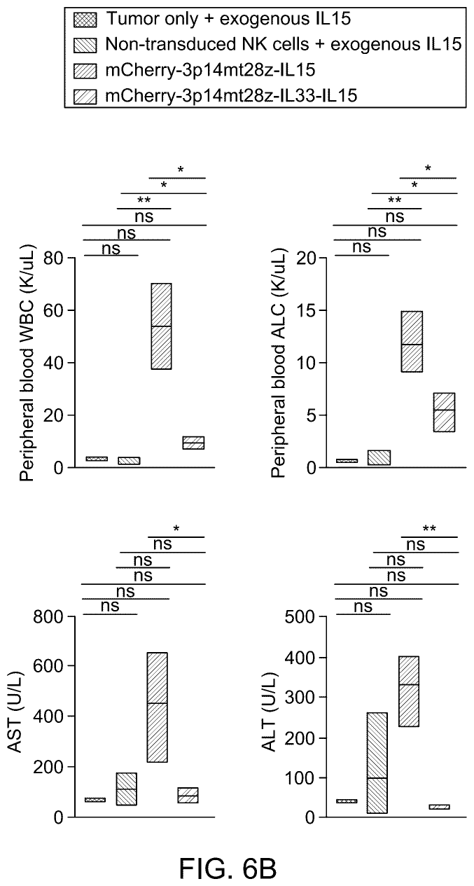

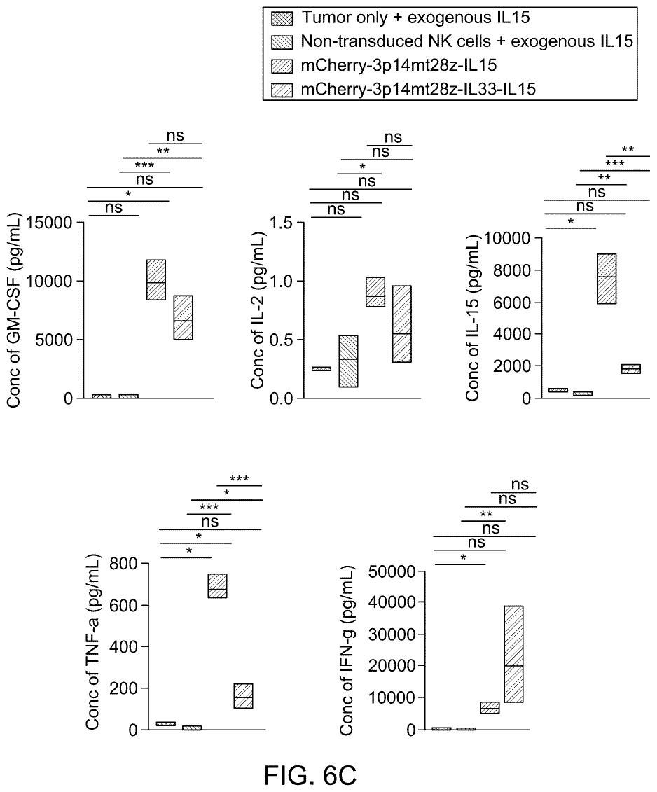

To further evaluate the toxicity associated with NK cells expressing mCherry-3p14mt28ζ-IL-15 but not mCherry-3p14mt28ζ-IL-33-IL-15, necropsies of 3 tumor-bearing mice treated with each type of NK cells, exogenous IL-15 alone, or non-transduced NK cells with exogenous IL-15 on day 17 after initial tumor engraftment were performed (the day prior to death in pilot studies). Mice given exogenous IL-15 but no NK cells or non-transduced NK cells and IL-15 (FIG. 6A) had evidence of large atypical round cell infiltrate, consistent with leukemia cells, in multiple tissues, but no other significant findings.

Mice that received CAR-modified NK cells secreting IL-15 (mCherry-3p14mt28ζ-IL-15) had severe infiltrates of small round cells, consistent with NK cells, in the spleen, liver, and lungs, and less severe similar infiltrates in the bone marrow, lymph nodes, and kidneys (FIG. 6A). In the liver and lungs, these infiltrates were associated with significant tissue injury and necrosis. These NK cells were observed in the peripheral blood in very high numbers on CBC (FIG. 6B). These mice had moderately elevated serum aspartate aminotransferase (AST) and alanine aminotransferase (ALT) (FIG. 6B) and did not have evidence of leukemia cells except in the salivary glands of one mouse. Additionally, two mice in this group had evidence of focal necrosis of the pituitary gland, which was not observed in other groups. The cause of this lesion is not clear, as no evidence of NK or leukemia cells was observed in the pituitary. Two mice in this group also showed mild acute tubular necrosis in the kidneys.

Mice that received CAR-modified NK cells secreting IL-15 fused to IL-33 (mCherry-3p14mt28ζ-IL-33-IL-15) had significantly milder infiltrate of small round cells, consistent with NK cells, in the spleen, liver, and lungs, and no such infiltrates in the bone marrow, lymph nodes, and kidneys (FIG. 6A). In the liver and lungs, no evidence of tissue injury or necrosis was associated with the infiltrates. These cells were observed in lower numbers in the peripheral blood on CBC (FIG. 6B), and similarly did not have evidence of leukemia cells except in the mesenteric lymph node and mesentery of one mouse.

To further evaluate this toxicity, serum cytokine levels were measured (FIG. 6C). GM-CSF, IL-2, TNF-a, and IL-15 levels were highest in the serum of mice treated with CAR-modified NK cells secreting IL-15, and IFN-γ was highest in those treated with CAR-modified NK cells secreting IL-15-IL-33, indicating that IL-33 is functional but not toxic.

### Discussion

The present example developed and evaluated CD33-directed CAR-modified peripheral blood-derived NK cells for the treatment of AML. Peripheral blood-derived NK cell transduction and expansion protocols were developed, addressing two of the major obstacles in engineering NK cells from this source. The present example shows findings of promising in vitro and in vivo efficacy of these NK cells, further modified to express IL-15 and IL-33, support their continued development toward clinical translation for the treatment of CD33-expressing AML.

Although NK cells inherently kill AML based on signals from activating and inhibitory cell surface receptors, it was demonstrated that modification with a CD33-directed CAR (3p14mt28ζ) improves short- and long-term cytotoxicity, serial killing, and NK cell activation against AML using transduced NK cells from 4 independent NK cell donors with varied KIR haplotypes in vitro. It was hypothesized that these CAR-modified NK cells are superior to other previously developed products such as CD33-CAR NK-92 cells, which pose the added risk of using a parental cell line originally derived from a lymphoma patient in an immunosuppressed recipient, and membrane-distal CD33-targeting CAR-NK cells transduced using lentiviral vectors, which result in lower preclinical efficacy as compared to membrane-proximal targeting CAR constructs and lower transduction efficiencies.

In in vivo experiments, NK cells modified with the complete (3p14mt28ζ) CAR construct conferred similar survival extension to NK cells expressing the control (3p14mtDEL) CAR-DEL construct, which includes the CD33-directed scFv but not the cytosolic domain of CD28 and CD3ζ. This finding is surprising because the signaling domains should be essential for linking the antigen-cell binding event to a cascade of intracellular molecular events. As non-transduced NK cells did not extend survival, the scFv may facilitate binding of NK cells to target cells in vivo, promoting NK cell function even without an intracellular domain. This phenomenon was likely not observed in vitro because of the already close proximity of targets and effectors in in vitro assays. Nonetheless, without being bound by any theory, it is believed that, in order to show the properties of 3p14mt28ζ, 3p14mtDEL is a better experimental control than unmodified NK cells used in previous CAR-modified NK cell preclinical evaluation because CAR modification is a stress to NK cells and affects viability and proliferation.

The presently disclosed results showed that the antitumoral efficacy of NK cells expressing CD33-directed CAR can be further augmented by the addition of IL-15, as NK cells modified with CAR constructs including IL-15 alone or IL-15 fused to IL-33 eliminated or significantly reduced leukemia burden. However, excessive IL-15 was toxic in in vivo model, consistent with prior published results (Ilias et al., J Immunother Cancer. 2021; 9 (12): e003894; Liu et al., Leukemia. 2018; 32 (2): 520-531). This excessive IL-15 production led to massive NK cell proliferation and significant toxicity that most severely targeted the liver and lungs. Addition of IL-33 to this CAR construct significantly attenuated this toxicity, while retaining similar efficacy. Therefore, to counteract these potential toxicities in humans, IL-33 (or potentially another cytokine such as IL 12 or IL18) or a suicide mechanism must be included in CAR constructs for NK cell modification.

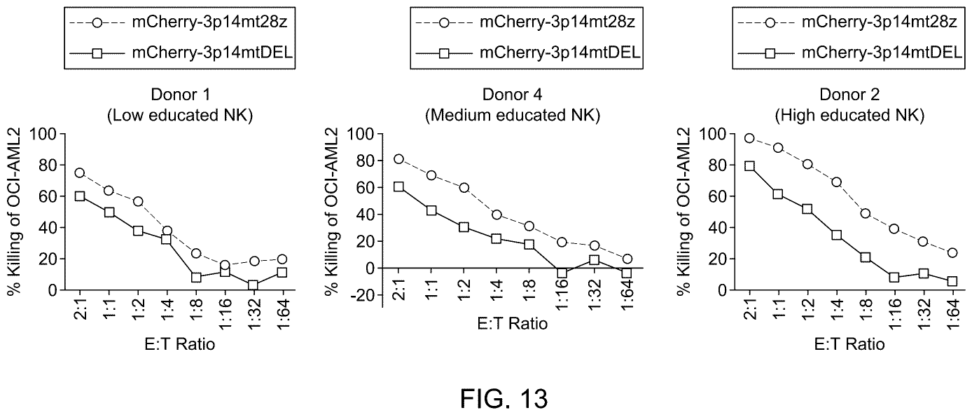

CAR-NK cells exert cytotoxicity that is non-CAR-mediated, as demonstrated by the modest killing of tumor targets by mCherry-3p14mtDEL and non-transduced NK cells. NK cells from the 4 different donors used in this study had differing baseline activity against each AML cell line, likely secondary to NK cell education (FIG. 13 and Tables 2 and 3). In the present example, CAR modification increased the cytotoxicity of all NK cells, but more highly educated NK cells have higher baseline cytotoxicity, likely attributable to the missing-self phenomenon. This may prove to be an advantage for NK cells over T cells in CAR-driven immunotherapy, especially for heterogenous diseases such as AML, as the intrinsic capacity of NK cells to recognize and target tumor cells remains intact, making disease escape through downregulation of the CAR target antigen less likely than it is with CAR T cells. One could potentially exploit this property further by selecting donors for NK-CAR production based on KIR-ligand mismatch with the recipient or KIR haplotype B gene content, as shown in stem cell transplantation (Cooley et al., J Immunol. 2014; 192 (10): 4592-4600).

Using readily accessible healthy donors and GMP-compliant procedures for robust expansion, multiple clinical doses of CAR-NK cells can be generated from a single apheresis. This overcomes many of the current limitations of CAR T cell therapy production. In the present example, NK cells transduced with a CAR incorporating CD28 showed marked antitumor activity in vitro. This success was limited in vivo, suggesting that the addition of other NK-cell specific costimulatory domains, such as CD16, DAP10, DAP12, DNAM1, or 2B4 to CAR constructs for NK cells should also be evaluated. Although the present example showed comparable cytotoxicity between cryopreserved and fresh NK cells, further evaluation is warranted, and cryopreservation protocols should be developed prior to clinical translation. Lastly, trogocytosis-mediated fratricide and tumor escape are potential concerns that have been observed in anti-CD19 CAR-modified NK cells and may be of concern in the presently disclosed anti-CD33 CAR-modified NK cells as well.

In conclusion, a novel approach to immunotherapy using engineered peripheral blood-derived NK cells has been developed. NK cells expressing a CD33-directed CAR in combination with an IL-15-IL-33 fusion can be produced and show promising efficacy both in vitro and in vivo.

### Embodiments of the Presently Disclosed Subject Matter

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. An immunoresponsive cell comprising a chimeric receptor that binds to an antigen without delivering: (1) an activation signal; and (2) a co-stimulatory signal to the immunoresponsive cell;
wherein the chimeric receptor comprises an extracellular antigen-binding domain, a transmembrane domain, and an intracellular polypeptide.

2. The immunoresponsive cell of claim 1, wherein
(a) the transmembrane domain comprises a CD8 polypeptide, a CD28 polypeptide, a CD3g polypeptide, a CD4 polypeptide, a 4-1BB polypeptide, an OX40 polypeptide, an ICOS polypeptide, a CD84 polypeptide, a CD166 polypeptide, a CD8a polypeptide, a CD8b polypeptide, a ICAM-1 polypeptide, a CTLA-4 polypeptide, a CD27 polypeptide, a CD40 polypeptide, a NKGD2 polypeptide, a PD-1 polypeptide, a LAG-3 polypeptide, a 2B4 polypeptide, a BTLA polypeptide, a DAP12 polypeptide, a DAP10 polypeptide, a CD16 polypeptide, a DNAM1 polypeptide, or a combination thereof; and/or
(b) the intracellular polypeptide comprises a CD28 polypeptide, a CD4 polypeptide, a 4-1BB polypeptide, an OX40 polypeptide, an ICOS polypeptide, a CD84 polypeptide, a CD166 polypeptide, a CD8a polypeptide, a CD8b polypeptide, a ICAM-1 polypeptide, a CTLA-4 polypeptide, a CD27 polypeptide, a CD40 polypeptide, a NKGD2 polypeptide, a PD-1 polypeptide, a LAG-3 polypeptide, a 2B4 polypeptide, a BTLA4 polypeptide, a DAP12 polypeptide, a DAP-10 polypeptide, a CD16 polypeptide, a CD266 polypeptide, or a combination thereof.

3. The immunoresponsive cell of claim 1, wherein the intracellular polypeptide comprises a CD28 polypeptide.

4. The immunoresponsive cell of claim 1, wherein the intracellular polypeptide comprises or consists of the amino acid sequence set forth in SEQ ID NO: 16.

5. The immunoresponsive cell of claim 1, wherein the intracellular polypeptide lacks a CD32 polypeptide.

6. The immunoresponsive cell of claim 1, wherein the antigen is a tumor antigen selected from CD33, CD19, carbonic anhydrase IX (CAIX), carcinoembryonic antigen (CEA), CD8, CD7, CD10, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinases Erb-B2,3,4 (erb-B2,3,4), folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13R□2), □-light chain, kinase insert domain receptor (KDR), Lewis Y (LeY), L1 cell adhesion molecule (LICAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, NKCS1, EGFIR, EGFR-VIII, CD99, CD70, ADGRE2, CCR1, LILRB2, PRAME CCR4, CD5, CD3, TRBC1, TRBC2, TIM-3, Integrin B7, ICAM-1, CD70, Tim3, CLEC12A, or ERBB.

7. The immunoresponsive cell of claim 1, wherein the chimeric receptor is recombinantly expressed and/or expressed from a vector.

8. The immunoresponsive cell of claim 1, wherein the immunoresponsive cell is a cell of the lymphoid lineage or a cell of the myeloid lineage.

9. The immunoresponsive cell of claim 8, wherein the cell of the lymphoid lineage is selected from the group consisting of a T cell, a B cell, a Natural Killer (NK) cell, and a dendritic cell.

10. The immunoresponsive cell of claim 9, wherein the immunoresponsive cell is a NK cell or a NKG2C+CD8+ T cell.

11. The immunoresponsive cell of claim 10, wherein said cell is derived from an induced pluripotent stem cell.

12. The immunoresponsive cell of claim 1, wherein the immunoresponsive cell further comprises: (a) a first cytokine selected from the group consisting of an IL-15 polypeptide or a functional fragment thereof, an IL-33 polypeptide or a functional fragment thereof, an IL-18 polypeptide or a functional fragment thereof, an IL-12 polypeptide or a functional fragment thereof, an IL-2 polypeptide or a functional fragment thereof, an IL-4 polypeptide or a functional fragment thereof, an IL-7 polypeptide or a functional fragment thereof, an IL-9 polypeptide or a functional fragment thereof, and an IL-21 polypeptide or a functional fragment thereof; and/or
(b) a cytokine receptor selected from the group consisting of an IL-15 receptor polypeptide, a ST2 polypeptide, an IL-18 receptor polypeptide, an IL-12 receptor polypeptide, an IL-2 receptor polypeptide, an IL-4 receptor polypeptide, an IL-7 receptor polypeptide, an IL-9 receptor polypeptide, and an IL-21 receptor polypeptide.

13. The immunoresponsive cell of claim 1, wherein the immunoresponsive cell further comprises an antigen-recognizing receptor that targets a second antigen; wherein the second antigen-recognizing receptor is a chimeric antigen receptor (CAR), a T cell receptor (TCR), a chimeric ligand receptor, a chimeric co-stimulating receptor (CCR), or a combination thereof.

14. A nucleic acid encoding a chimeric receptor that binds to an antigen and that does not deliver an activation signal and a co-stimulatory signal to an immunoresponsive cell; wherein the chimeric receptor comprises an extracellular antigen-binding domain, a transmembrane domain, and an intracellular polypeptide.

15. The nucleic acid of claim 14, wherein the intracellular polypeptide comprises a CD28 polypeptide.

16. The nucleic acid of claim 14, wherein the intracellular polypeptide lacks a CD35 polypeptide.

17. A vector comprising the nucleic acid of claim 14.

18. A lipid nanoparticle comprising the nucleic acid of claim 14.

19. A composition comprising the cell of claim 1.

20. The composition of claim 19, which is a pharmaceutical composition further comprising a pharmaceutically acceptable excipient.

21. A method of reducing tumor burden in a subject, treating a subject having a relapse of a neoplasm, and/or treating and/or preventing a neoplasm in a subject, the method comprising administering the cell of claim 1 or a pharmaceutical composition thereof.

22. The method of claim 21, wherein the method reduces the number of tumor cells, reduces tumor size, and/or eradicates the tumor in the subject.

23. The method of claim 21, wherein the neoplasm or tumor is a blood cancer or a solid tumor.

24. The method of claim 23, wherein the blood cancer is multiple myeloma, myeloid disorder, leukemia, or lymphoma.

25. The method of claim 24, wherein
(a) the leukemia is acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, or B cell prolymphocytic leukemia;
(b) the lymphoma is Hodgkin's lymphoma, non-Hodgkin's lymphoma, B-cell non-Hodgkin's lymphoma, or T-cell non-Hodgkin's lymphoma; or
(c) the solid tumor is selected from the group consisting of renal cell carcinoma, non-small-cell lung cancer, lung adenocarcinoma, lung squamous cell carcinoma, lung neuroendocrine carcinoma, small-cell lung cancer, pancreatic cancer, breast cancer, astrocytoma, glioblastoma, laryngeal/pharyngeal carcinoma, EBV-associated nasopharyngeal carcinoma, and ovarian carcinoma.

26. The method of claim 21, wherein the subject is a human.

27. A method for producing a modified cell, the method comprising introducing into a cell the nucleic acid of claim 14.

28. A kit comprising the cell of claim 1 or a pharmaceutical composition thereof.
